(19) United States
(12) Patent Application Publication
(10) Pub. No.: US 2006/0053501 A1
(43) Pub. Date: Mar. 9, 2006

(54) Method for detecting Nipah virus and method for providing immunoprotection against Henipa viruses

(75) Inventors: Marie-Claude George Courbot, Lyon (FR); Fabian Wild, Lyon (FR); Vincent Deubel, Paris (FR); Robin Buckland, Lyon (FR); Hugues Contamin, Brindas (FR); Thong Wong Kum, Kuala Lumpur (MY); Philippe Loth, Villemhamme (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: INSTITUT PASTEUR, Paris Cedex (FR)

(21) Appl. No.: 11/131,263

(22) Filed: May 18, 2005

Related U.S. Application Data
(63) Continuation of application No. 10/945,948, filed on Sep. 22, 2004, now abandoned.
(60) Provisional application No. 60/584,472, filed on Jul. 2, 2004.
(60) Provisional application No. 60/504,225, filed on Sep. 22, 2003.

(51) Int. Cl. A01K 67/027; C12Q 1/70; C12Q 1/68

(52) U.S. Cl. 800/9; 435/5; 435/6; 800/14

(57) ABSTRACT

The present invention provides an animal model for monitoring Nipah virus infection, a method for the quantitative detection and rapid characterization of Nipah virus RNA in a sample, a composition which can be used to provide immunoprotection in an individual as well as monoclonal antibodies which neutralize Nipah and Hendra virus and can be used for prophylaxis, treatment, and/or prevention.

### CROSS-REFERENCE TO RELATED APPLICATIONS

The present application claims the benefit of U.S. application 60/584,472 filed Jul. 2, 2004 and U.S. application 60/504,225 filed Sep. 22, 2004, the contents of both are incorporated herein by reference.

### BACKGROUND OF THE INVENTION

1. Field of the Invention

The present invention relates to a detection method for Nipah virus in a sample and a method for providing immunoprotection against Nipah and Hendra virus infections.

2. Description of the Background

Nipah virus emerged in Malaysia in 1998, resulting in important morbidity and mortality in both pig and man (Chua, 2000, Science. 288:1432-5). The zoonotic infection most probably involved Pteroid bats (flying foxes) as natural hosts that transferred Nipah virus to the pig population via their urine or remains of partially eaten fruit (Chua, et al 2002, Microbes Infect. 4:145-51; Chua, K. B. 2003, J. Clin. Microbiol. 26:265-275). Pig farmers and abattoir workers who were in direct contact with the infected animals were the most targeted population. Pig-to-human transmission through close contact appeared to be the most usual route of contamination, with the pig playing the part of an amplifying host for the virus (Parashar, et al 2000, J Infect Dis. 181:1755-9; Mohd Nor et al 2000, Rev Sci Tech Off Int Epiz. 19(1):160-5). Infected pigs mainly suffered a respiratory disease with less than 5% mortality, whereas 105 deaths were recorded among 265 human patients who developed severe acute febrile encephalitic syndrome with a quarter of the survivors having residual neurological side effects (Goh, et al 2000, New Engl J. Med. 342:1229-35; Chong, et al 2002, Can J Neurol Sci. 29:83-7; Lee, et al 1999, Ann Neurol. 46:428-32).

Nipah virus is a member of the subfamily Paramyxovirinae in the Paramyxoviridae family. Its biological properties and genomic organization classify the virus and the closely-related Hendra virus, in a new genus called henipavirus (Wang, et al 2000, J Virology. 74:9972-9979). Nipah virus contains a single-stranded RNA of about 18,000 nucleotides associated with the viral proteins of the replicative complex (the nucleoprotein (N), the phosphoprotein (P), and the polymerase (L)) enclosed by a lipid bilayer envelope containing the attachment protein (G) and the fusion protein (F) (Chua, 2000, Science. 288:1432-5; Wang, et al 2001, Microbes and Infection 3, 279-287; Chan, et al 2001, J Gen Virol. 82:2151-5).

The broad distribution of the Pteropus sp. old world fruit bats extends southeast from the western islands of the Indian Ocean, across southeast Asia and northeast Australia to the southwest islands of the Pacific. Little is known about factors potentially responsible for the emergence of Henipaviruses (Morse, S. S. 1995. Emerg Infect Dis. 1(1):7-15; Field, et al 2001, Microbes Infect. 3:307-314). The presence of Nipah virus has already been demonstrated in Cambodia in 2002 since anti-NIV antibodies have been found in fruit bats (Olson, et al 2002, Emerg Infect Dis. 8:987-988) and presumably in Bangladesh in 2001, 2003 and more recently in 2004 (ProMed 2002 Nipah-like virus—Bangladesh (2001, 2004): Archive numbers 20020830.5187-20040423.1127) (ICDDR,B 2003, Health and Science Bulletin, ISSN 1729-343×, vol. 1:1-6). If an efficient program to prevent or treat Nipah virus infection in man is to be developed, it will be necessary to define the viral antigens which are important in inducing protective responses and to formulate potential immunoprophylactic treatments.

There is also a priority for the development of specific serologic and virologic diagnostics for an accurate surveillance of henipavirus circulation (Daniels, et al 2001, Microbes Infect. 3:289-95). Rapid diagnosis of the viruses in the zoonotic cycle or in patients with acute encephalitis would help the adoption of appropriate measures at the medical, veterinarian and environmental levels. Real-time polymerase chain reaction methods based on TaqMan™ technology have recently been developed for testing viral load in infectious diseases and in cell culture (Heid, et al 1996, Genome Res. 10:986-94; Klein, et al 2003, J Virol Methods. 107(2):169-75).

In nature, paramyxoviruses can infect both man and animals. Often, viruses preferentially infect one species and grow poorly in a second. Thus a virus that grows poorly in the second species can be used to create a “Jenner” type vaccine. In the same manner, by the use of modern biotechnology the antigens of a virus that is a human pathogen can be expressed from an equivalent animal virus in order to induce protective responses (Schmidt, et al 2002. J. Virol. 76:1089-1099; Yunus, et al 1999. Arch Virol. 144:1977-1990). In certain cases, when paramyxoviruses cross the species barrier to infect man they become more virulent. The natural host of Hendra and Nipah viruses is probably the fruit bat (Chua, K. B., et al 2002. Microbes Infect. 4:145-51; Field, H., et al 2001. Microbes Infect. 3:307-314; Yob, et al 2001. Emerg Infect Dis. 7:439-441) but in 1994 and in 1998 in Australia horses became infected by Hendra virus and in 1998 in Malaysia Nipah virus infected pigs. In both cases, virus was amplified in the second animal species and this led to human infection. The severity of the disease caused by Nipah in pigs (more than a million culled) and in humans (40% fatality) had great economic and social consequences. Ribavirin was tried on some patients but with little significant results (Chong, H. T., et al 2001. Ann Neurol. 49:810-813; Snell, N. J. 2001. Expert Opin Pharmacother. 2:1317-13124). No Nipah-specific antivirals were available to combat the epidemic and their production remains a priority if effective measures are to be taken when future epidemics occur.

In view of the above, there is a need to provide several tools to monitor the pathophysiology linked to Henipavirus infection (e.g. animal model and quantitative method for quantification of viral load). There is also a a need to provide a simple, reliable, specific and sensitive assay for quantitatively detecting Nipah-like or Hendra-like viruses in a sample. Furthermore, in light of the inherent danger resulting from Nipah and Hendra virus infections, there also remains a need to provide treatment or protective immunity to those requiring such protection. Thus, identification of an animal model reproducing the human disease and amenable for anti-viral and vaccine trials is required. Moreover, innovative approaches are needed to prevent or treat henipavirus infection.

### SUMMARY OF THE INVENTION

Accordingly, the present invention provides a hamster model that reproduces the pathology and pathogenesis of acute human Nipah infection.

Another object of the present invention also provides a method for the quantitative detection and rapid characterization of Nipah virus RNA in a sample.

Another object of the present invention is an immunogenic composition comprising Nipah virus glycoproteins and a pharmaceutical acceptable carrier and further wherein the immunogenic composition is a vaccine.

Another object of the present invention is a method of protecting an individual against a Nipah virus infection comprising administering Nipah virus glycoproteins or polynucleotides which encode the glycoproteins to said individual in an amount sufficient to induce an immune response in said individual.

Another object of the present invention is an immunoreactive composition for protecting or curing an individual against a Nipah virus infection comprising of administred antibodies directed against the attachment and/or the fusion glycoproteins of Nipah virus or cross-reactive in the Henipavirus genus.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1. Survival graphs of 7-14 week old hamsters infected by Nipah virus via two routes. The lethal dose of virus killing fifty percent of hamsters (LD50) by intraperintoenal and intranasal route was, respectively, 270 pfu and 47,000 for each animal.

FIG. 2. Vascular and parenchymal pathology in acute Nipah infection. A: Large artery in liver showing focal, transmural fibrinoid necrosis with surrounding inflammation. B: Myocardial necrosis with adjacent inflammation. C: Multiple endothelial multinucleated syncytium in pulmonary artery. D: Viral RNA was demonstrated in in endothelial syncytia and vascular smooth muscle in the same lung. E: Necrosis and karyorrhexis in a cerebral vessel. F: Viral antigen localized in the endothelium ans smooth muscle in a meningeal blood vessel.

FIG. 3. Cerebral pathology in acute Nipah infection. A: Small vessel vasculitis characterized by mild inflammation in the vicinity of infected neurons. B: Focal areas of parenchymal ischemi, infarction and oedema. C: Neurons with eosinophilic inclusions. D: Immunolocalization of viral antigens to neurons in the nucleus, cytoplasm, and processes near a vasculitis vessel. E: Viral antigens localized to ependymal lining and neurons. F: Neurons demonstrating viral RNA in the cytoplasm.

FIG. 4. A & B: Inflammation of the lung parenchyma associated with vasculitis and thrombotic blood vessels. C: Glomerultis characterized by thrombotic plugs, inflammation and syncytial formation at the periphery of the glomerulus. D: Viral antigens were detected in a tubule of glomerulus. E: Viral antigens found in the epithalium covering the papilla in the kidney. F: Viral antigens demonstrated in lymphoid cells of the white pulp in the spleen.

FIG. 5. Detection of Nipah virus RNA by the TaqMan™ real time RT-PCR. Amplification plots were realized on ten fold dilutions of Nipah virus RNA extracted from Nipah virus stock. Tests were performed in duplicate from undiluted to 1/10⁶.

FIG. 6. Standard curve obtained with ten fold serial dilutions of Nipah virus RNA. Ct values calculated from results obtained in FIG. 5 are plotted against the log of the initial starting quantity of infectious virus (pfu/ml). The threshold is 0.289601.

FIG. 7. Standard curve for Nipah virus RNA transcripts showing the threshold cycle Ct plotted against the log of initial amounts of Nipah RNA transcripts. Three amplification plots were performed using different RNA transcripts.

FIG. 8. Nipah virus infection and syncytia formation of Vero cells. Cells infected with a MOI of 0.01 were treated at day 1(a) and 2(b) after infection and tested by immunofluorescence for the presence of viral antigens. The cytopathic effect was visualized by the formation of cell syncytia containing high numbers of nuclei. Nuclei were stained with propidium iodide.

FIG. 9. Evolution of the number of infectious Nipah virus and Nipah virus RNA detected in infected cell supernatants by plaque assays and real-time RT-PCR assay at days 1, 2, 3 and 4 after infection.

FIG. 10. FACScan analysis of HeLa cells infected with vaccinia virus (VV) recombinants expressing either the G or F glycoproteins of NiV. HeLa cells were infected with either VV-NiV.G or F or a control VV at a moi of 0.1 pfu/cell for 16 hr and the expression of the glycoproteins measured at the surface of the cells with a polyclonal monospecific antiserum to either the G or F glycoproteins.

FIG. 11. Induction of fusion by co-expression of the Nipah virus G and F glycoproteins. Hela cells were infected with VV-NiV recombinants expressing either the G or F glycoproteins or doubly infected with both as in FIG. 10. The cells were then examined for viral expression by immunoflorescence and also the induction of fusion.

FIG. 12. Protection of hamsters from a lethal challenge of Nipah virus by vaccination with VV recombinants expressing the Nipah virus G and/or F glycoproteins. Hamsters were vaccinated twice at a 1 month interval with either VV.NiV G or F or both and challenged with Nipah virus 3 months after the last immunization (7-8 animals/group). Animals were examined daily.

FIG. 13. Antibody responses after vaccination with VV recombinants and after challenge with Nipah virus. The hamsters were bled after immunization and also at periods after the challenge with Nipah virus. Antibody levels were measured by (A) neutralization and (B) by ELISA.

FIG. 14. Passive protection of hamsters against a lethal Nipah virus infection. Antibody was raised in hamsters against the VV recombinants expressing either G or F and pooled sera either against the individual glycoprotein or an equal mixture of each were inoculated i.p. (0.2 ml/animal) 2 hr prior to challenge with Nipah virus. A second inoculation of antisera (0.2 ml) was given 24 hr later. The animals were challenged with Nipah virus and observed for 43 days.

FIG. 15. The immune response of hamsters challenged with Nipah virus in the presence of passively administered polyclonal monospecific anti Nipah virus sera. The hamsters from FIG. 14 were bled at intervals and the sera examined for anti-Nipah virus antibodies by ELISA.

### DETAILED DESCRIPTION OF THE INVENTION

Unless specifically defined, all technical and scientific terms used herein have the same meaning as commonly understood by a skilled artisan of molecular biology.

The present invention provides for the first time the demonstration that golden hamster can be infected with Nipah virus injected by either intranasal or intraperitoneal route and die with encephalitic syndromes characteristic of Nipah virus in infected humans. Moreover, the lesions observed in the necropsies show similar pathology compared to those observed in human tissue samples. In particular, the lesions show virus tropism for vascular endothelial cells which form syncytia, and lead to vasculitis, thrombosis, ischemia, infarctus, and perivascular inflammation in a similar way as observed in human infections (Wong et al., Am. J. Pathol. 2002. 161:2153-2167). It has also been demonstrated that neurons of the central nervous system are target cells for Nipah virus. Viral antigens and RNA were localized in both vascular and extravascular tissues including neurons, lung, kidney, and spleen. Finally, virus was isolated from urine of infected animals, providing a relevant way to follow up the presence of virus replication without invasive procedure. Thus, in one embodiment of the present invention, a golden hamster model of Henipavirus infection is provided, which hamster is infected by at least one Henipavirus such as Nipah virus and Hendra virus. This golden hamster model reproduces the majority (i.e., greater than 50%) of the symptoms observed in an infected human. The model can be advantageously used as a substitute for human and non-human primates for, e.g., diagnosis, virus production, virus phenotype discrimination, and therapeutic and prophylactic assessments.

The present invention also provides for the first time, a versatile, reliable, and sensitive test to rapidly quantify Nipah virus RNA in cell culture and in biological samples. Inactivation of virus infectivity during the process of RNA extraction should allow any laboratory involved in surveillance and diagnosis of this virus to monitor the circulation of Nipah virus in endemic regions. This technique may also be of interest to quantify viral RNA molecules in tissue specimens. It has been described that Nipah virus may persist in humans and cause late onset encephalitis, or that it may relapse to cause resurgent encephalitis several months after the initial disease (Tan, et al 2002, Ann Neurol. 51:703-8). Although live virus could not be isolated from cerebro-spinal fluid at these late stages, the presence of Nipah virus was revealed by the demonstration of viral antigens in the brain.

Paramyxoviruses including Nipah and Hendra viruses, have two glycoproteins at the virus surface, the G and the F. The G glycoprotein is responsible for the attachment to the cellular receptor, whereas the F glycoprotein induces the fusion between the viral and cellular membranes. G and F act in concert to bring about fusion. The present inventors have confirmed this for the vaccinia expressed Nipah virus proteins showing that only co-infection i.e. G+F induced fusion. If antibodies are to block infection, then they should presumably block attachment of G to its receptor or the inhibition of the function of F to fuse the virus envelope with the cell membrane. Sera from hamsters immunized with either of the VV recombinants induced high antibody levels but relatively low neutralizing antibodies. In other paramyxoviruses, the response to the attachment protein often tends to be dominant but we found that the antibody responses to Nipah virus.F and Nipah virus.G were of the same order, confirming studies made in mice (Tamin, et al 2002. Virology. 296:190-200).

Basic scientific techniques, encompassed by the present invention are known. See, for example, Sambrook et al., Molecular Cloning: A Laboratory Manual, Third Edition, Cold Spring Harbor Laboratory, New York (1999) and various references cited therein.

“Isolated” refers to a material, i.e. a polynucleotide, separated out of its natural environment.

“Recombinant” refers to a genetically engineered polynucleotide or polypeptide prepared in vitro by cutting up polynucleotides and splicing together specific polynucleotide fragments.

“Polynucleotide” in general relates to polyribonucleotides and polydeoxyribonucleotides, it being possible for these to be non-modified RNA or DNA or modified RNA or DNA.

“Polypeptides” are understood as meaning peptides or proteins, which comprise two or more amino acids, bonded via peptide bonds.

As used herein, “inhibit”, “inhibiting” or “inhibition” includes any measurable reproducible reduction in the infectivity of a Henipavirus such as Nipah virus and/or Hendra virus in the subject patient.

The term “expression vector” refers to a polynucleotide that encodes the peptide of the invention and provides the sequences necessary for its expression in the selected host cell. Expression vectors will generally include a transcriptional promoter and terminator, or will provide for incorporation adjacent to an endogenous promoter. Expression vectors may be plasmids, further comprising an origin of replication and one or more selectable markers. In addition, expression vectors may be viral recombinants designed to infect the host, or integrating vectors designed to integrate at a preferred site within the host's genome. Examples of viral recombinants are Adeno-associated virus (AAV), Adenovirus, Herpesvirus, Poxvirus, Retrovirus, vaccinia virus and other RNA or DNA viral expression vectors known in the art. In a preferred embodiment, the expression vector is a viral vector and in a particularly preferred embodiment, the viral vector is a recombinant vaccinia virus.

The method of assaying in the present invention can employ reverse transcriptase-polymerase chain reaction (RT-PCR), in which PCR is applied in conjunction with reverse transcription. Typically, RNA is extracted from a sample tissue using standard techniques and is reverse transcribed to produce cDNA molecules. This cDNA is then used as a template for a subsequent polymerase chain reaction.

Once primer and template have annealed, a DNA polymerase is employed to extend from the primer, thus synthesizing a copy of the template. The DNA strands are then denatured and the process is repeated numerous times until sufficient DNA is generated to allow visualization using fluorescence, radionuclides, or other detectable moieties if attached to at least one of the primers or other means to visualize the amplified polynucleotide molecule, e.g., ethidium bromide staining or spectrophotometry.

Biological samples for use within such assays include blood, sera, urine, tissue biopsies, lymph node, peritoneal fluid, cerebrospinal fluid and prostate secretions, as well as other tissues, homogenates, and extracts thereof. Such biological samples may be prepared using any standard technique.

Polynucleotides that encode the Nipah virus and Hendra virus proteins (or a portion or other variant thereof) or that is complementary to such a polynucleotide, may be used within the methods provided herein. Polynucleotides may be single-stranded (coding or antisense) or double-stranded, and may be DNA (cDNA or synthetic) or RNA molecules. Additional coding or non-coding sequences may, but need not, be present within a polynucleotide of the present invention, and a polynucleotide may, but need not, be linked to other molecules and/or support materials.

Polynucleotides may be prepared using any of a variety of techniques. For example, a polynucleotide may be amplified via polymerase chain reaction (PCR) from cDNA. For this approach, sequence-specific primers may be designed based on the sequences provided herein, and may be purchased or synthesized. Other polynucleotides may be directly synthesized by methods known in the art, such as chemical synthesis.

Particularly preferred portions of a coding sequence or a complementary sequence are those designed as a primer to detect Nipah virus or other Henipavirus such as Hendra virus in a sample. Primers may be labeled by a variety of reporter groups or detectable moieties, such as radionuclides and enzymes, and are those comprising at least 15, 20, 25, or 30 consecutive nucleotides of the Nipah virus polynucleotides, e.g., SEQ ID NOS: 8 and 17, or their complements, as appropriate, described herein, for example, the sequence shown in SEQ ID NO:1. Primers for PCR are those comprising at least 15, 20, 25, or 30 consecutive nucleotides of the Nipah virus polynucleotides or their complements, as appropriate described herein, for example, the sequences as shown in SEQ ID NOS:2 and 3. In a preferred embodiment, the primers used for reverse transcription and subsequence amplification specifically target the nucleocapsid region of the Nipah virus genomic RNA.

The polynucleotides and polypeptide sequences of various Nipah virus isolates are known and constituents of the Nipah virus include a nucleocapsid (NC), a matrix, a polymerase, an attachment glycoprotein, and P/V/C fusion proteins. Examples of such polynucleotides include those available from GenBank under the accession numbers AJ564622, AJ564621, AF376747, AF212302, AY029768, and AY029767. Further, those sequences shown as SEQ ID NOS:8 and 17 in the Sequence Listing also correspond to Nipah virus polynucleotides.

Likewise, the amino acid sequences of Nipah virus polypeptides have been described, for example, see GenBank entries AJ564622, AJ564621, AF376747, AF212302, AY029768, and AY029767. Further non-limiting examples of specific viral components include polymerase-SEQ ID NOS:9, 18, 28, and 30; Attachment protein-SEQ ID NO:10; Fusion protein (F)-SEQ ID NOS:11 and 20; Matrix protein-SEQ ID NO:12, 21, and 27; C protein—SEQ ID NO:13; V protein—SEQ ID NO:14, 25 and 26; Phosphoprotein—SEQ ID NO:15, 22, and 24; and Nucleocapsid—SEQ ID NOS:16, 23, 31 and 32; Glycoprotein—SEQ ID NO:19 and 29.

The polynucleotides and polypeptide sequences of various Hendra virus isolates are known and constituents of the Hendra virus. Examples of such polynucleotides include those available from GenBank under the accession numbers AF017149 and AF 010304. Further, those sequences shown as SEQ ID NOS:33 and 45 in the Sequence Listing also correspond to Hendra virus polynucleotides.

Likewise, the amino acid sequences of Hendra virus polypeptides have been described, for example, see GenBank entries AF017149 and AF 010304. Further non-limiting examples of specific viral components include nucleocapsid-SEQ ID NO:34; phosphoprotein—SEQ ID NOS:35 and 42; nonstructural protein V—SEQ ID NOS:36 and 43; nonstructural protein C—SEQ ID NOS:37 and 44; matrix protein—SEQ ID NO:38; fusion protein—SEQ ID NO:39; glycoprotein—SEQ ID NO:40; and polymerase—SEQ ID NO:41. In one embodiment, the proteins that are at least 70%, preferably at least 80%, more preferably at least 90% identical to the Nipah virus or Hendra virus amino acid sequences described herein can be employed in the present invention. In another embodiment, the Nipah virus or Hendra virus proteins that can be used are those that are encoded by polynucleotide sequence with at least 70%, preferably 80%, more preferably at least 90%, 95%, and 97% identity to the Nipah virus or Hendra virus coding sequence, these polynucleotides will hybridize under stringent conditions to the coding polynucleotide sequence of the Nipah virus polynucleotide sequences described herein. The terms “stringent conditions” or “stringent hybridization conditions” includes reference to conditions under which a polynucleotide will hybridize to its target sequence, to a detectably greater degree than other sequences (e.g., at least 2-fold over background). Stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides), for example, high stringency conditions include hybridization in 50% formamide, 1 M NaCl, 1% SDS at 37° C., and a wash in 0.1×SSC at 60 to 65° C. (see Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York (1993); and Current Protocols in Molecular Biology, Chapter 2, Ausubel, et al., Eds., Greene Publishing and Wiley-Interscience, New York (1995)). Amino acid and polynucleotide identity, homology and/or similarity can be determined using the ClustalW algorithm, MEGALIGN™, Lasergene, Wisconsin)

The proteins having identity or those proteins encoded by the polynucleotides which hybridize to the polynucleotides described herein preferably retain at least 20%, preferably 50%, more preferably at least 75% and/or most preferably at least 90% of the biological activity of wild-type Nipah virus or Hendra virus protein activities—the amount of biological activity include 25%, 30%, 35%, 40%, 45%, 55%, 60%, 65%, 70%, 75%, 80%, 85%, 95%; and all values and subranges there between. Furthermore, they can also have 100% or more of the biological activity relative to wild-type Nipah virus or Hendra virus activity—the amount of biological activity including at least 105%, at least 110%, at least 125%, at least 150%, and at least 200%. The percentage of amino acid similarity between virus proteins inside the Henipavirus genus and in particular between the envelope glycoproteins underlines the capacity of each of these proteins to induce antibodies with cross-reactive and cross-protective reactivities.

The Nipah virus or Hendra virus proteins may be purified to substantial purity by standard techniques well known in the art, including selective precipitation with such substances as ammonium sulfate, column chromatography, immunopurification methods, and others. See, for instance, R. Scopes, Protein Purification: Principles and Practice, Springer-Verlag: New York (1982).

The present invention also encompasses methods of treatment or prevention of a disease caused by the Nipah virus and also to Hendra virus and to any member of the Henipavirus genus, by mounting an immune response. In the method of treatment, the administration of the immunoreactive compositions described herein may be for either “prophylactic” or “therapeutic” purpose. When provided prophylactically, the immunoreactive compositions are provided in advance of any symptom. The prophylactic administration of the immunoreactive compositions serves to prevent, improve, and/or reduce the severity of any subsequent infection or disease. When provided therapeutically, the immunoreactive compositions are provided at (or shortly after) the onset of a symptom of infection or disease. Thus the present invention may be provided either prior to the anticipated exposure to a disease causing agent or disease state or after the initiation of the infection or disease.

As used herein, the subject patient that would benefit from the administration of the formulations described herein includes any animal which can benefit from protection against viral infection. In a preferred embodiment, the subject patient is a human patient, a horse, or a pig which are amplifying hosts and are of economical interest.

The virus polypeptides can be used prophylactically as vaccines. The vaccines of the invention contain as an active ingredient an immunogenically effective amount of the binding or fusing domain polypeptide or of a recombinant virus as described herein. The immune response may include the generation of antibodies; activation of cytotoxic T lymphocytes (CTL) against cells presenting peptides derived from the virus polypeptides, or other mechanisms well known in the art. See e.g. Paul Fundamental Immunology Second Edition published by Raven press New York (incorporated herein by reference) for a description of immune response. Useful carriers are well known in the art, and include, for example, thyroglobulin, albumins such as human serum albumin, tetanus toxoid, polyamino acids such as poly(D-lysine:D-glutamic acid), influenza, hepatitis B virus core protein, hepatitis B virus recombinant vaccine.

The DNA or RNA encoding the virus polypeptides may be introduced into patients to obtain an immune response to the polypeptides which the polynucleotide encodes. For example, in this embodiment an expression vector, as described herein, is used and is inoculated into a subject patient to induce an immune response.

An amount sufficient to accomplish immunoprotection or prophylaxis is defined as an “immunogenically effective dose.” Amounts effective for this use will depend on the composition, the manner of administration, the weight and general state of health of the patient.

The term “unit dose” as it pertains to the inoculum refers to physically discrete units suitable as unitary dosages for mammals, each unit containing a predetermined a quantity of the recombinant antigens or polynucleotides encoding the recombinant antigens calculated to produce the desired immunogenic effect in association with the required diluent. The specifications for the novel unit dose of an inoculum of this invention are dictated by and are dependent upon the unique characteristics of the recombinant virus and the particular immunologic effect to be achieved.

The inoculum is typically prepared as a solution in tolerable (acceptable) diluent such as saline, phosphate-buffered saline or other physiologically and/or pharmaceutically acceptable diluent and the like to form an aqueous pharmaceutical composition.

The route of inoculation may be intravenous, intramuscular, subcutaneous, intradermal and the like, which results in eliciting a protective response against Nipah virus. The dose is administered at least once. Subsequent doses may also be administered.

In providing a mammal with the immunogenic compositions of the present invention, preferably a human, the dosage of administration will vary depending upon such factors as the mammal's age, weight, height, sex, general medical condition, previous medical history, disease progression, tumor burden and the like.

After immunization the efficacy of the vaccine can be assessed by production of antibodies or immune cells that recognize the antigen, as assessed by specific lytic activity or specific cytokine production or by tumor regression. One skilled in the art would know the conventional methods to assess the aforementioned parameters.

Immunostimulatory agents or adjuvants can be used to improve the host immune responses may also be included in the immunogenic compositions. Adjuvants have been identified that enhance the immune response to antigens. Aluminum hydroxide and aluminum phosphate are commonly used as adjuvants in human and veterinary vaccines. Other extrinsic adjuvants and other immunomodulating materials can elicit immune responses to antigens. These include saponins complexed to membrane protein antigens to produce immune stimulating complexes (TSCOMS), pluronic polymers with mineral oil, killed mycobacteria in mineral oil, Freund's complete adjuvant, bacterial products, such as muramyl dipeptide (MDP) and lipopolysaccharide (LPS), as wall as monophoryl lipid A, QS 21 and polyphosphazene.

In a preferred embodiment, Nipah virus glycoproteins (G and F) are used separately and in an alternative preferred embodiment the G and F glycoproteins are used in combination in the immunogenic compositions of the present invention. In a preferred embodiment, the immunogenic composition is an expression vector carrying the Nipah virus proteins which upon inoculation express the proteins to elicit an immune response, e.g., recombinant vaccinia virus expressing the Nipah virus glycoproteins and more preferred is the vector that expresses the G and F glycoproteins of Nipah virus.

A bank of monoclonal antibodies (Mabs) against the Nipah virus G and F proteins and which neutralize Nipah virus infectivity in vitro have also been developed. Furthermore, certain of the anti-Nipah virus F proteins neutralize Hendra virus. Thus, another embodiment of the present invention is recombinant hybridomas producing the antibodies against Henipavirus G and F proteins as well as vaccine vector recombinants expressing Henipavirus G and F proteins

Non-limiting examples of the vaccinia vector recombinants and hybridomas include the recombinant vaccinia virus expressing Nipah G protein was deposited at CNCM on Sep. 16, 2003 under the no I-3086; the recombinant vaccinia virus expressing Nipah F protein was deposited at CNCM on Sep. 16, 2003, under the number I-3085; the hybridoma N°1.7 anti-Nipah virus G protein with neutralizing activity against Nipa virus was deposited at the CNCM on Sep. 7, 2004; the hybridoma N°3.B10 anti-Nipah virus G protein with neutralizing activity against Nipa virus was deposited at the CNCM on Sep. 7, 2004; the hybridoma N°35 anti-Nipah virus F protein with neutralizing activity against Nipah and Hendra virus was deposited at the CNCM on Sep. 7, 2004; and he hybridoma N°3 anti-Nipah virus F protein with neutralizing activity against Nipah and Hendra virus was deposited at the CNCM on Sep. 7, 2004.

### EXAMPLES

### Example 1

### A Golden Hamster Model of Henipavirus

A recent outbreak of a novel paramyxovirus subsequently named Nipah virus (NiV) infected hundreds of patients in Malaysia causing severe morbidity, and a mortality rate of about 40% (Chua et al. 2000. Science 288:1432-1435). Patients developed symptoms ranging from fever and headache to a severe acute febrile encephalitic syndrome. Although the majority of symptomatic patients who survived the acute infection eventually recovered without serious sequelae, a small number were readmitted with relapsed encephalitis months and years later Tan et al. Ann Neurol. 2002.51:703-708). The clinical features and pathogenesis of relapsed encephalitis were found to be distinct from acute NiV encephalitis. Pig-to-human transmission through close contact is now well-established, with the pig playing the part of an amplifying host for the virus (Parashar et al. J Infect Dis. 2000. 181:1755-1759). The natural host is very likely to be the fruit bat since NiV has been isolated from bat's urine recently Chua et al. Microbes Infect. 2002. 4:145-151). Thus, the NiV outbreak represents the most serious viral zoonosis that has emerged from bats recently (Eaton, Microbes Infect. 2001. 3:277-278).

Based on studies of NiV-infected human tissues, the pathology and pathogenesis of NiV infection is beginning to be understood (Wong et al. Am J Pathol. 2002. 61:2153-2167). In acute NiV infection, present evidence suggests that following primary viral replication, viremia occurred spreading the virus systemically. Blood vessels became infected resulting in widespread vasculitis, which led to thrombosis, vascular occlusion, ischemia and/or microinfarction in multiple organs, affecting the central nervous system (CNS) most severely (Wong et al. Am J Pathol. 2002. 61:2153-2167). Extravascular parenchymal tissues, most notably neurons, were also susceptible to infection. It has been postulated that a combination of CNS ischemia and/or microinfarction, and direct neuronal infection may contribute to the severe neurological manifestations seen in acute NiV infection (Wong et al. Am J Pathol. 2002. 61:2153-2167).

Attempts to further understand the early pathogenesis of acute NiV infection were hampered by the lack of an animal model. Present knowledge of the pathology and pathogenesis of acute NiV infection relates to the late stages of the disease since the studies were based on human autopsies. Naturally and experimentally infected animals including pigs and cats that have been studied so far showed vasculitis but not the typical encephalitis found in human NiV infection, and thus may not be suitable as models (Hooper et al. 2001. Microbes Infect. 3:315-322). The anti-viral ribavirin, which was used as an empirical therapy in infected patients and reported to be effective, has yet to be fully evaluated in animal experiments (Chng et al., Ann Neurol. 2001. 49:810-813). Likewise, other anti-viral agents and newly-developed vaccines could not be tested for their potential usefulness in NiV infection due to the lack of a good model. Controlled transmission studies in animal models could be conducted to investigate viral infectivity and the routes of infection.

In this study we investigated several animal species as potential models for acute NiV infection, and identified the golden hamster (Mesocricetus auratus) as a suitable model. The pathological lesions in hamster infected intranasally and intraperitoneally were characterized by various approaches, and showed a high degree of similarity to those found in the human disease. We also attempted to correlate virus isolation and viral genome detection in various infected organs with pathological changes found therein.

Materials and Methods

Virus Stock and Titration

NiV isolated from the cerebrospinal fluid of a patient was received in the BSL-4 “Jean Merieux” laboratory in Lyon, France, from Dr K B Chua and Dr S K Lam (University of Malaya, Kuala Lumpur, Malaysia) after 2 passages in Vero cells. Virus stock was obtained after a third passage on Vero cells conducted under physical containment level 4.

After 1-2 days of infection when Vero cells showed fusion and syncytia formation, the supernatant was harvested for virus. Virus stock was titrated in 6-well plates by incubating 200 μl of serial 10 times dilution of supernatant in each well (containing 10⁶ Vero cells per well) for 1 hr at 37° C. The cells in each well were washed twice with Dulbecco's minimum essential medium (DMEM), and 2 ml of 1.6% carboxymethylcellulose in DMEM containing 2% fetal calf serum were added to each well. The plates were incubated for 5 days at 37° C., and the wells were washed with phosphate buffer pH 7.4 (PBS), fixed with 10% formalin for 20 min, washed and stained with methylene blue. The virus titer in the supernatant after 24 hr of infection at a multiplicity of infection (MOI) of 0.01 was 2×10⁷ plaque forming units (pfu)/ml.

Animal Infection Experiments

Altogether 3 series of animal studies were done. In the first study, preliminary testing for susceptibility to NiV infection was done on 2 groups of animals comprising 5 mice, 2 guinea pigs and 2 hamsters each. Four week-old, female Swiss mice (Charles River, L'Arbresle, France), 4 month-old, male Hartley guinea pigs (Charles River), and 2 month-old male golden hamsters (Janvier, Le Fenest St Isle, France) were used in this experiment in which each group was inoculated either by the intranasal (IN) or the intraperitoneal (IP) route. For the IN route, 30 μl of virus stock (6×10⁵ pfu) was given to each animal, while for the IP route 0.5 ml (10⁷ pfu) was inoculated. The animals were observed for signs of infection. The animals were housed in ventilated containment equipped with Hepa filters in the animal room of the BSL-4 lab. We followed the French regulations for handling animals, and the strict procedures imposed for work in high security BSL-4 containment.

Based on the results of the first study, a second study was then performed on adult hamsters (7-14 weeks old) using IN and IP inoculation routes to determine the lethal doses needed to kill 50% of the animals (LD₅₀). Groups of 6 hamsters were infected with 10-fold dilutions of NiV stock and observed twice daily over 4 weeks.

In order to investigate the possibility of on-going reinfection between animals housed together in the same cage contributing to mortality, a third study was done. In this study 2 hamsters infected by IP route with 10⁵ pfu of virus were placed 3 days postinoculation in the same cage as 4 other uninfected hamsters. The animals were observed, and retroorbital sinus blood samples obtained for serology after 30 days.

Suitable tissue specimens from the first and second studies including blood, brain, lung, heart, liver, spinal cord, spleen and kidney were collected from a total of 12 hamsters who died recently (≦12 hours) or were terminally moribund. The latter were anethetized with ketamine and xylazine, and exsanguinated by cardiac puncture and necropsied. Urine was collected from the bladder whenever possible. Animals discovered dead after more than 12 hours were not studied.

Tissues were frozen at −80° C. for viral culture and reverse transcription-polymerase chain reaction (RT-PCR) analysis. For histopathologic studies, tissues were fixed in 10% buffered formalin for at least 15 days before routine tissue processing and paraffin embedding outside the BSL-4 laboratory. Tissues from the nasal passage and cervical lymph nodes were also dissected out from formalin-fixed carcasses for routine processing and paraffin embedding only. For electron microscopy (EM), fresh or formalin-fixed tissues were fixed in 3% glutaraldehyde in 0.1 M phosphate buffer pH 7.4 for a few hours and transferred to phosphate buffer. Similarly, tissues for immunoelectronmicroscopy (IEM) were fixed in 2% paraformaldehyde/0.05% glutaraldehyde, and transferred to buffer. In addition, EM and IEM tissues which were initially not formalin fixed, were gamma-irradiated (2×10⁶ rads) to further ensure non-infectivity.

Blood samples were collected by cardiac puncture at necropsy or obtained from the retroorbital sinus in surviving animals in the second study 4 weeks after infection. The NiV doses causing mortality of 50% of the hamsters were calculated based on the method of Reed and Muench.

Virus Isolation and Titration

The quantity of infectious virus particles was measured in urine and other tissues by plaque titration in Vero cells. A small fragment of each organ was mechanically-crushed (Mini-beadbeater; Biospec, Bartlesville, USA) twice for 30 seconds each in a 2 ml tube containing 0.5 ml of sterile glass beads and 0.5 ml of DMEM. The tubes were centrifuged at 3000 rpm for 5 min at 4° C., and 200 μl of serial dilutions of the supernatant were layered on 6-well plates of Vero cells for virus titration.

Nipah Antibody Testing

Sera of infected hamsters were tested individually by enzyme-linked immunosorbent assay (ELISA) for the presence of NiV antibodies. Crude extracts of NiV antigens were prepared from infected Vero cells at an MOI of 0.01 pfu/cell for 24 hours. The cells were washed with PBS and lysed in PBS containing 1% Triton X100 (10⁷ cells/ml) at 4° C. for 10 min. The cell lysate was sonicated twice for 30 seconds each to full cell destruction and centrifuged at 5000 rpm at 4° C. for 10 min. The supernatant was frozen at −80° C. Non-infected Vero cells were similarly treated to prepare an antigen control. Cross-titration of the Nipah antigens was performed with serum from a convalescent, NiV-infected patient to determine the antigen titer corresponding to the dilution showing the highest O.D. reading.

Reverse Transcription-Polymerase Chain Reaction

Total RNA was extracted from 20 μl of serum and urine, and from mechanically-crushed, fresh frozen tissues using an RNA extraction kit (QIAamp Viral RNA Mini Kit; Qiagen Inc., Valencia, Calif., USA). About 2 μg of the extract was used in an RT-PCR protocol (Titan One Tube RT-PCR System; Roche Diagnostics, Mannheim, Germany) to detect the presence of NiV nucleoprotein (N) gene. Specific primers were previously published (Chua et al. Science. 2000. 288:1432-1435).

Light Microscopy

Formalin-fixed, paraffin-embedded tissues were microtomed 3 μm thick, placed on glass slides, and stained with hemalin-phloxine-safranin stain for light microscopy.

Immunohistochemistry (IHC)

Tissue sections of 3 μm thickness were placed on silanized slides and dewaxed by xylene and graded ethanol washes. Antigen was retrieved by thermic treatment in pH 6.0 citrate buffer at 96-98° C. for 40 min. After cooling to room temperature (20° C.), the sections were incubated at 20° C. throughout, and sequentially as follows, with PBS washes in between steps: (a) 4% bovine serum albumin/10% goat serum (GS) in PBS, 15 min; (b) rabbit-raised, polyclonal anti-NiV antibody, 1:500, 1 hr; (c) biotinylated, goat anti-rabbit secondary antibody, 30 min (Dako, Trappes, France); (d) 0.09% H₂O₂ in PBS; (e) horseradish peroxidase-linked streptavidin and diaminobenzidine substrate according to the manufacturer's protocol (Dako, Trappes, France). The slides were counterstained in hematoxylin and mounted in an aqueous medium (Aquamount, Merck Eurolab, Strasbourg, France).

In Situ Hybridization (ISH)

For ISH, digoxigenin (DIG)-labeled riboprobes were generated from the 228 bp, RT-PCR product using the Nipah virus specific primers (Chua et al. Science. 2000. 288:1432-1435). This fragment was cloned in the pdrive cloning vector (Qiagen PCR cloning kit, Qiagen Inc., Valencia, Calif., USA) according to the manufacturer's protocol. Plasmids containing the correct insert in both orientations were linearized with the restriction endonuclease Hind III, and transcribed to produce sense and anti-sense riboprobes using the DIG RNA labeling kit (Roche Diagnostics, Mannheim, Germany). The riboprobes were treated with DNase (15 min, 37° C.) then purified by ethanol precipitation before use.

Dewaxed tissue sections were pretreated with 0.2 N HCl (20 min, 20° C.) followed by 0.1 mg/ml proteinase K in 100 mM Tris/50 mM EDTA, pH 8.0 buffer (15 min, 37° C.). After 2 PBS washes, the slides were incubated overnight at 45° C. in a moist chamber (Hybaid Omnislide) with 1:50 to 1:100 dilution of riboprobes in filtered hybridization solution containing 45% formamide, 6×SSC (1×SSC=0.15 M sodium chloride, 0.015 M sodium citrate, pH 7.0), 5× Denhardt's solution, 100 μg/ml denatured salmon sperm, 100 μg/ml yeast tRNA and 10% dextran sulphate.

Sequential post-hybridization steps included (a) 6×SSC (3×20 min, 45° C.); (b) 2×SSC (10 min, 20° C.); (c) 100 mM Tris, pH 7.5/150 mM NaCl buffer (1 min, 20° C.); (d) The same Tris/NaCl buffer with 2% GS and 0.1% Triton (30 min, 20° C.). The slides were then incubated with alkaline phosphatase-conjugated, anti-DIG Fab fragments (Roche diagnostics, Mannheim, Germany) diluted 1:1000 in Tris/NaCl/GS/Triton buffer in a moist chamber (overnight, 20° C.). The reaction was stopped by washes with Tris/NaCl (pH 7.5) buffer (3×10 min) and 100 mM Tris, pH 9.0/150 mM NaCl/50 mM MgCl₂ buffer (1 min) before incubation with the Tris/NaCl/MgCl₂ buffer containing NBT/BCIP solution (Roche Diagnostics, Mannheim, Germany) according the manufacturer's protocol. The colour reaction was stopped using 10 mM Tris, pH 8.0 buffer after about 45 min. The slides were counterstained with haematoxylin and coverslipped in an aqueous medium.

Animal Infection Experiments: Survival and LD₅₀

In the first study, none of the Swiss mice inoculated by either IN or IP route developed any clinical signs. Only Hartley guinea pigs that were infected by IP route, and therefore received 10⁷ infectious viral particles, showed transient fever and weight loss after 5-7 days but they recovered. Golden hamsters infected by both routes showed difficulties with movement and balance, and rapidly died 5-8 days after infection.

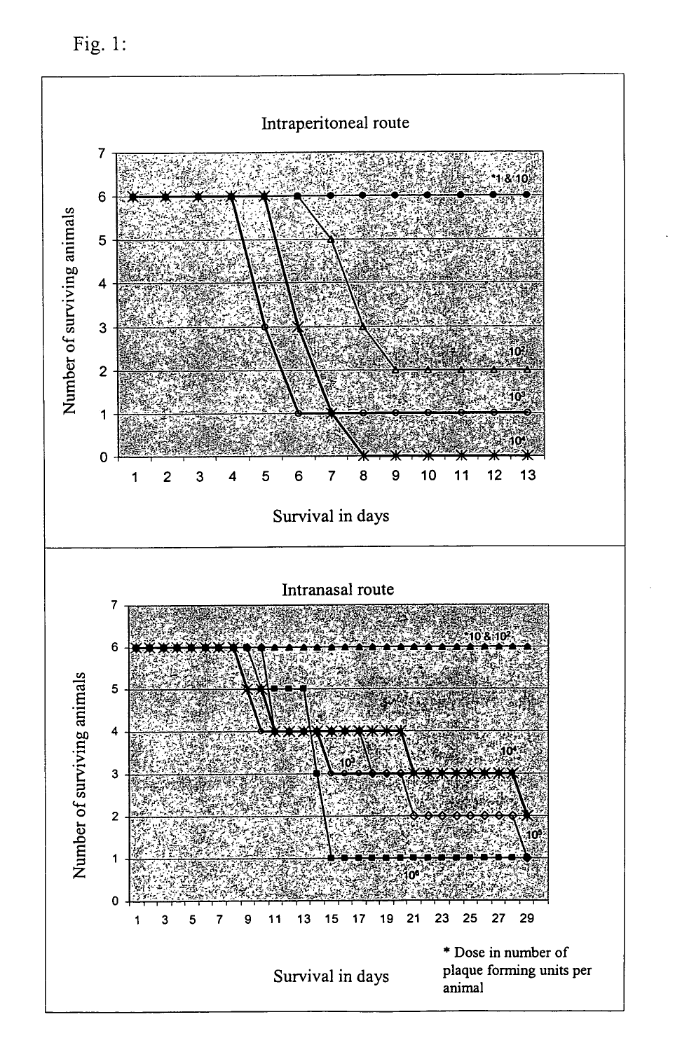

FIG. 1 shows the dose-survival graphs of hamsters in the second study that were inoculated with serial dilutions of viruses, viz., 1 to 10⁴ pfu by IP route and 10 to 10⁶ pfu by IN route. The time interval between infection and appearance of clinical signs and death were shorter in IP-infected hamsters. They died 5 to 9 days after infection and <24 hours after the appearance of tremor and limb paralysis. Conversely, the majority of IN-inoculated animals showed a progressive deterioration presenting with imbalance, limb paralysis, lethargy, muscle twitching and breathing difficulties in the final stages. The majority of animals died between 9 and 15 days. However, 6 animals died later, 1 at day 18, 2 at day 21 and 3 at day 29. The LD₅₀ of animals by IP and IN route was respectively 270 pfu and 47,000 pfu for each animal.

In animals surviving more than 30 days post-infection, and which were inoculated with lower viral doses (1 and 10 pfu/animal for IP route; 10 and 10² pfu/animal for IN route) there was no seroconversion (data not shown). In fact, none of these animals died or showed any signs of illness. In contrast, surviving animals infected with higher viral doses, and which were kept in the same cages as animals given the same doses and died, had high levels of antibody (data not shown). Nonetheless, these survivors showed no clinical signs of illness.

In the transmission study (third study) in which uninfected animals were housed together with infected animals, none of the uninfected animals showed evidence of disease or seroconversion (data not shown).

Viral Isolation and Viral Genome Detection

In general, RT-PCR of various animal specimens taken at autopsy showed that NiV viral genome could be detected in most tissues and urine (Table 2). Serum was the notable exception in that it was uniformly negative for viral genome. Because of this, viral culture was not attempted on serum. Where both these tests were performed, the range of tissues positive for viral culture correlated well with RT-PCR, although the percentage for positivity was lower for viral culture especially in intranasally infected hamsters.

Pathological Features

Blood Vessels

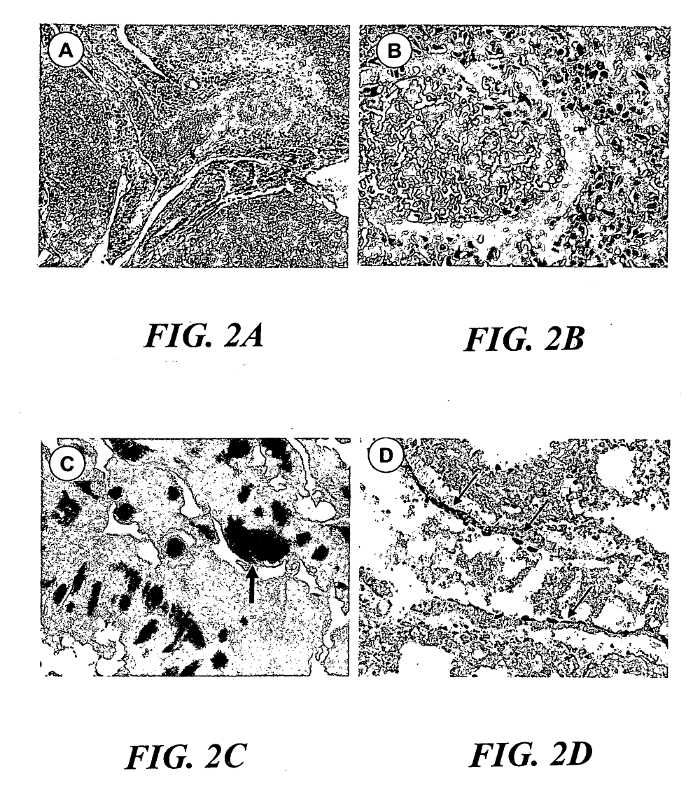

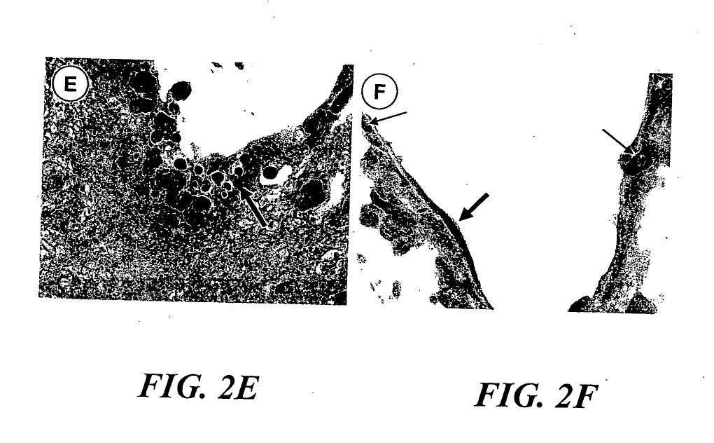

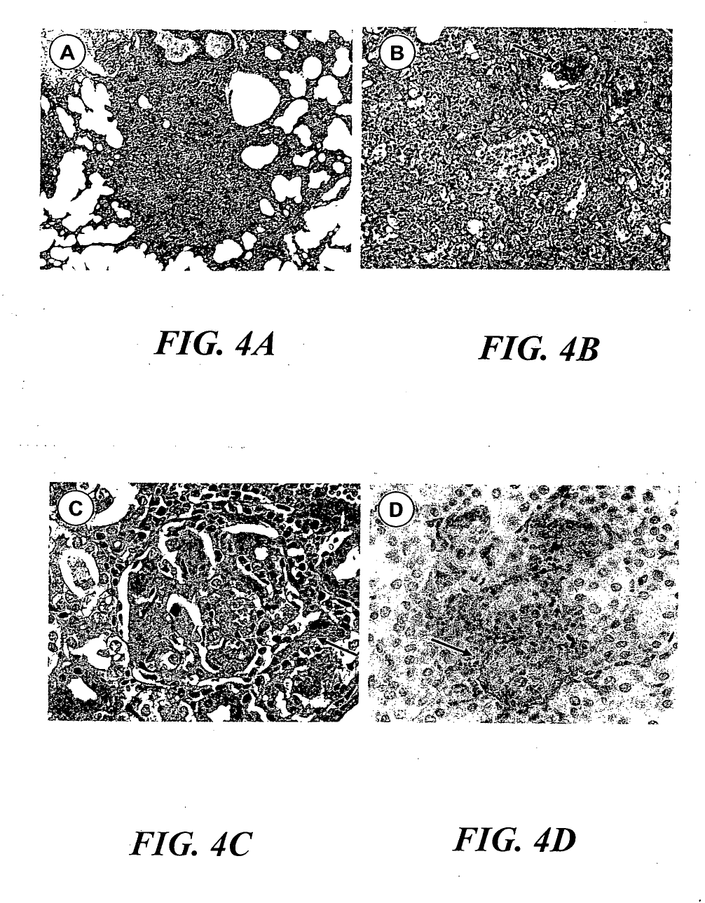

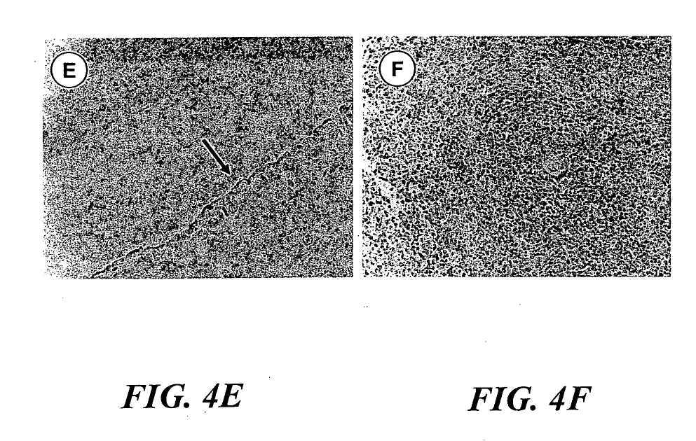

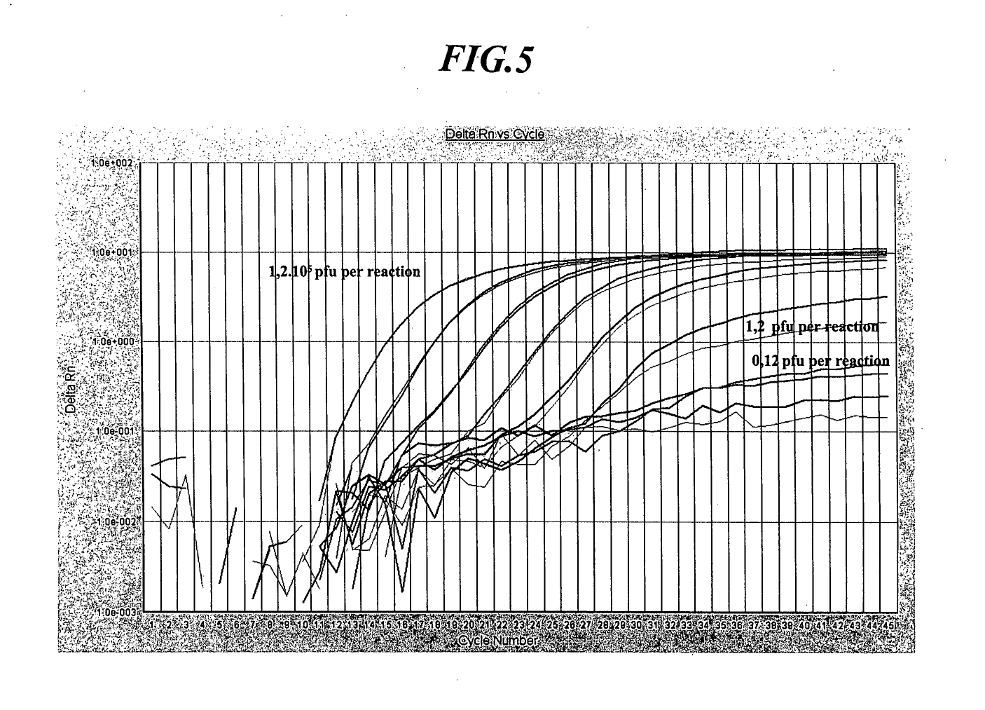

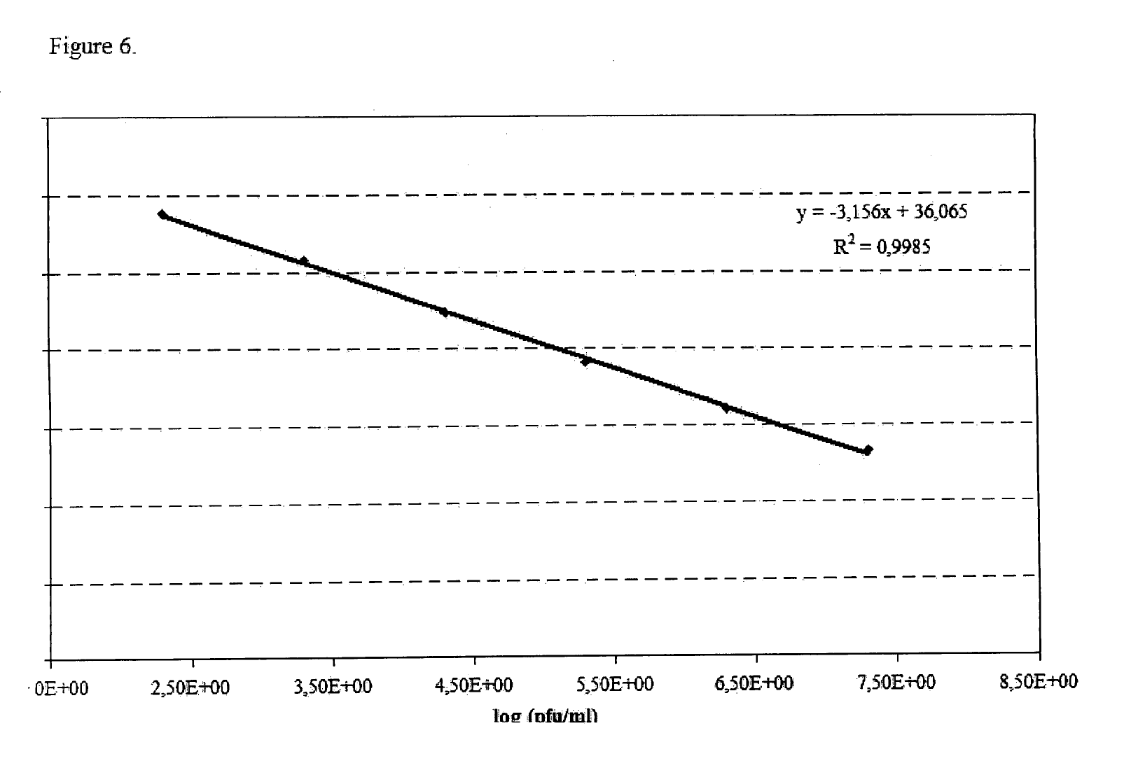

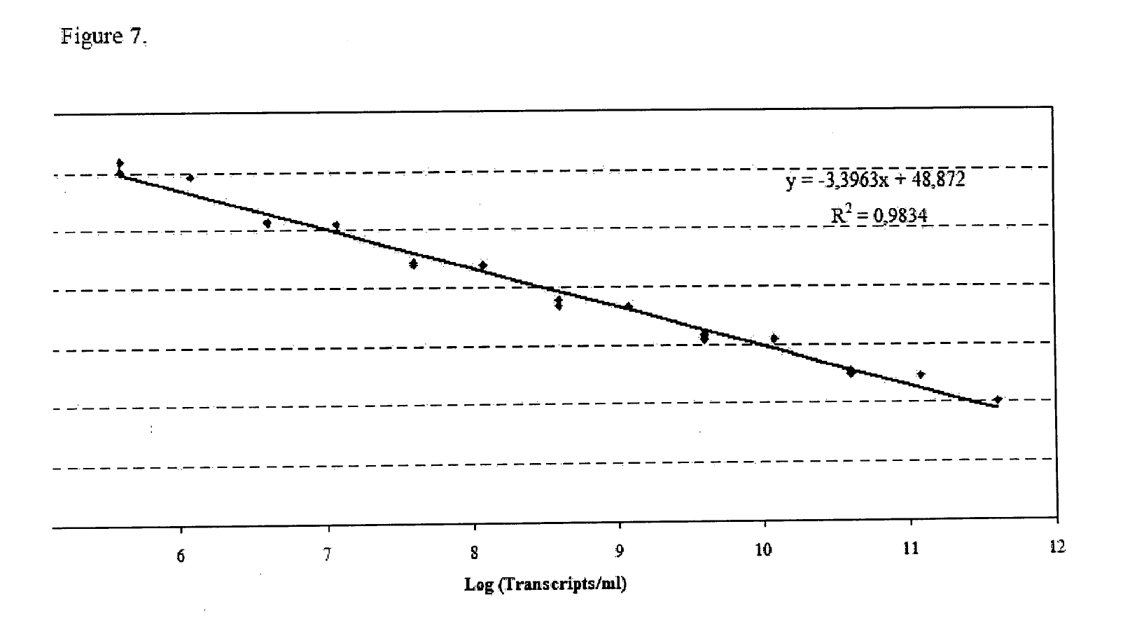

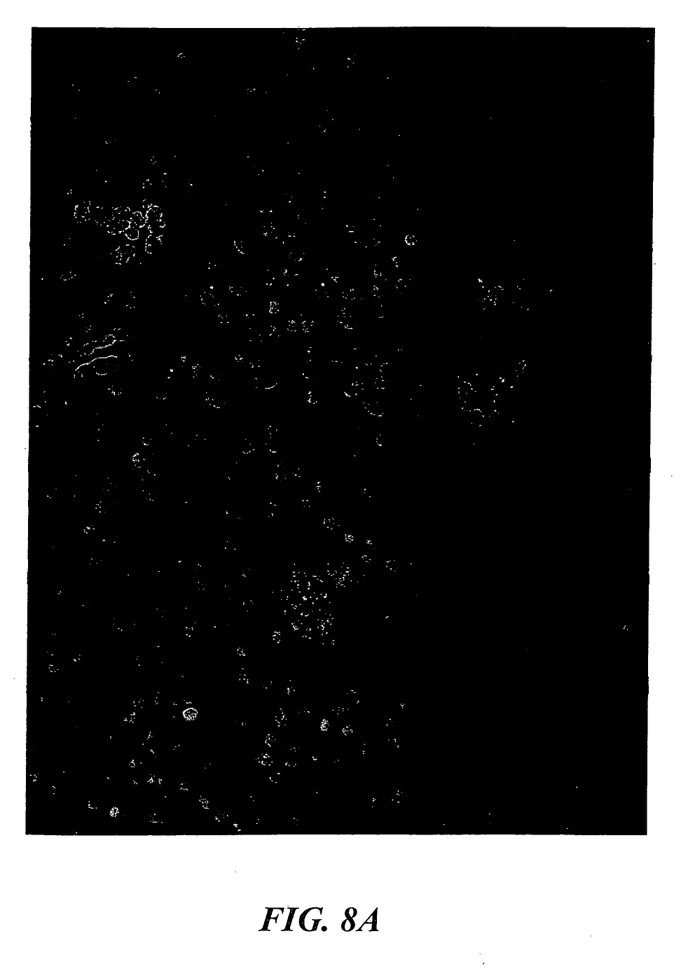

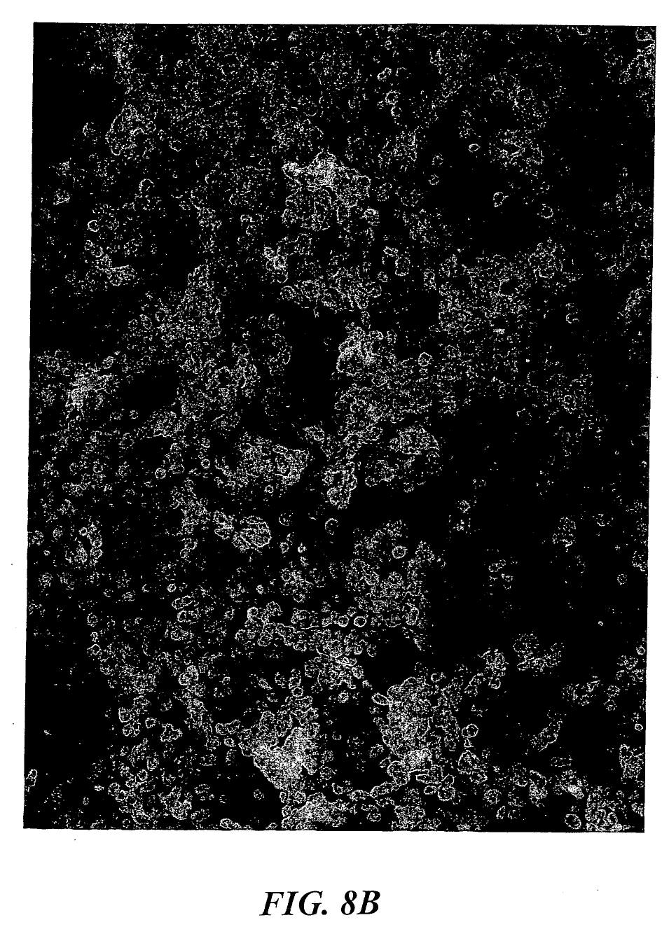

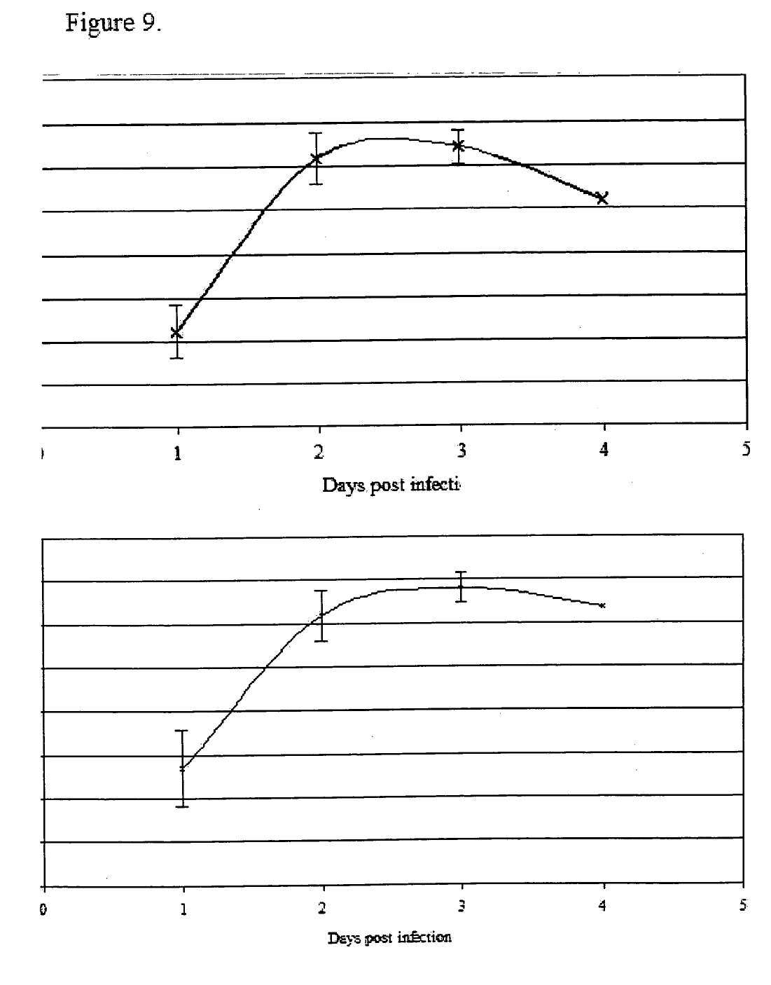

Vascular pathology was found in multiple organs including brain, lung, liver, kidney and heart. In large blood vessels the more florid changes were characterized by focal, transmural fibrinoid necrosis with surrounding inflammation (FIG. 2A). However, vasculitis may be more subtle with fewer inflammatory cells (FIG. 2E, 3A), and very focal nuclear pyknosis and karyorrhexis (FIG. 2E). Multinucleated syncytia arising from the endothelium were encountered in one hamster that died 8 days after intraperitoneal inoculation (FIG. 2C). Thrombosis could be found in the lumen of some vessels (FIG. 4B). Viral antigen and genome as demonstrated by IHC and ISH respectively localized to endothelial cells and syncytia, and underlying smooth muscle of the tunica media in blood vessels (FIG. 2D, F). Viral nucleocapsids were detected in the blood vessel wall.

Central Nervous System

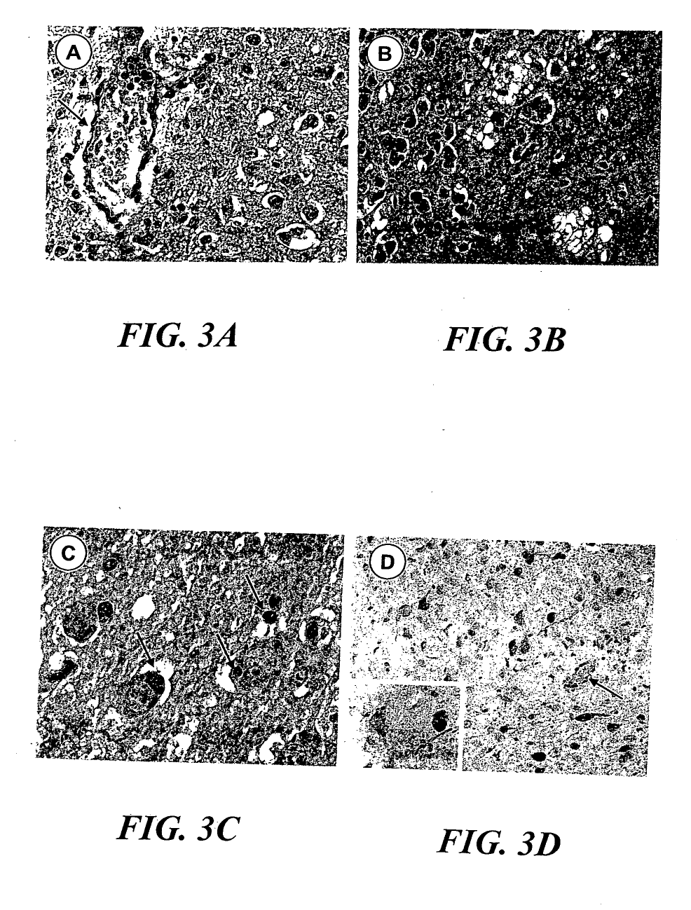

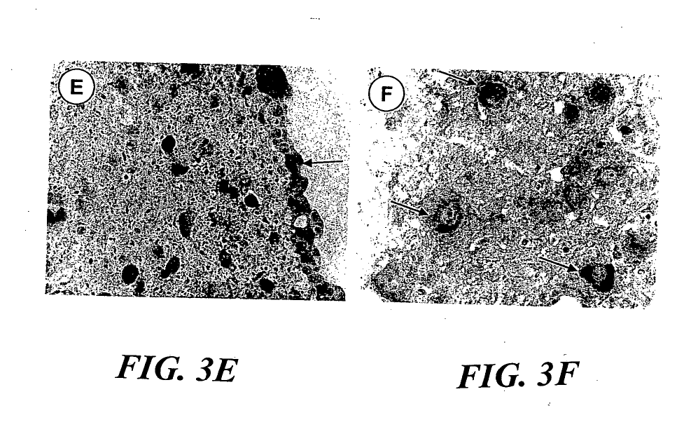

The brain was the most severely affected in terms of vascular and parenchymal lesions compared with other organs. Apart from vasculitis, the most striking features were in the neurons usually found in the vicinity of vasculitis. Affected neurons showed numerous eosinophilic inclusion bodies in the cytoplasm (FIG. 3C). These inclusions, as well as neuronal cytoplasm with no obvious inclusions, and neuronal processes, were often positive for both viral antigen and RNA (FIG. 3D-F). Ultrastructurally, these inclusions were composed of defined masses of filamentous nucleocapsids of the fuzzy type typically associated with paramyxoviruses (FIG. 5A). These inclusions were immunolabeled by NiV-specific antibodies (FIG. 5B). Nuclear inclusions could not be found but there was evidence of nuclear IHC positivity (FIG. 3D, inset).

Other parenchymal changes included focal areas with evidence of ischemia/infarction and edema (FIG. 3B). Parenchymal and meningeal inflammation were generally mild, and only occasionally were perivascular cuffing and neuronophagia observed. Rarely, IHC positivity was noted in ependymal lining (FIG. 3E), and in mononuclear cells found in the meninges and choroid plexus. The choroid plexus lining epithelium however was negative for viral antigen and genome. IHC and ISH positivity was not observed in the white matter.

Other Organs

In the lung, small discrete nodular or more confluent areas of parenchymal inflammation, often associated with vasculitic vessels, could sometimes be observed (FIG. 4A, B). Inflammatory cells consisted mainly of a varying mixture of macrophages, neutrophils and lymphocytes. Multinucleated giant cells and inflammatory cells positive for NiV by IHC and ISH were rare. Fibrinoid necrosis of lung parenchyma was not evident. Bronchitis, multinucleated syncytia or other evidence of NiV infection of bronchial epithelium were not found.

Glomerular lesions in the kidney were rare but the most florid lesions had thrombotic plugs in the glomerular capillaries, peripheral multinucleated syncytia, and surrounding inflammation (FIG. 4C). Viral antigen was detected only in the occasional glomerulus and tubule (FIG. 4D). In the kidney of several animals, the covering epithelium of the renal papilla that project into the calyces, consistently demonstrated the presence of viral antigen (FIG. 4E) but ISH was negative in the same epithelium.

The rare focus of necrosis was noted in the spleen but no vasculitis or multinucleated giant cells were observed. IHC and ISH were occasionally positive in periarteriolar lymphoid cells (FIG. 4F). There appeared to be no specific liver parenchymal lesions. In the heart, myocarditis associated with infarction was only rarely observed (FIG. 2B). No inflammation or viral antigen was detected in lymph nodes or nasal epithelium. Of the 3 animal species viz., mouse, guinea pig and hamster which were inoculated with NiV, the hamster appeared to be the most susceptible. Depending upon the route and dose most of the infected hamsters developed severe illness. Studies of tissues obtained from infected hamsters suggested that it is a suitable animal model for acute NiV infection, demonstrating most of the characteristics found in human acute NiV infection.

Hamsters could be infected by either IP or IN routes but infection by the IP appeared to kill animals faster than the IN route. Furthermore, far lower IP doses were required to kill the same number of animals as shown by the widely disparate LD₅₀ doses between IP and IN-infected animals. This is probably not surprising since IN-inoculated NiV presumably had to penetrate the mucosal barrier of the aero-digestive tract before infection could take place, whereas IP-inoculated NiV theoretically could enter the systemic circulation directly.

Histopathologic studies of infected hamster tissues showed that blood vessels, particularly those in the CNS, developed vasculitis characterized by necrosis and intramural inflammation. Evidence of direct viral infection of the vessel wall, including the endothelium and smooth muscle, was provided by the presence of endothelial multinucleated syncytia formation, and the detection of viral nucleocapsid, antigen and genome in the vascular wall. Most likely as a result of vasculitis, thrombosis and vascular obstruction occurred producing distal ischemia and microinfarction in the brain and heart. Blood vessels in the lung and kidney were also involved with vasculitis although to a lesser extent, and infarction was not obvious. These findings are similar to those found in human infection (Wong et al; Am J Path. 2002. 161:2153-2167) A notable exception could be vasculitis in the liver which was not reported in human infection.

In addition to ischemia and infarction, CNS neurons also showed evidence of infection by the presence of neuronal viral inclusions, antigen and genome. Viral inclusions found mainly in the cytoplasm consisted of typical paramyxoviral-type nucleocapsids. The findings in blood vessels, parenchyma and neurons of the CNS makes it the major target in acute NiV infection, and this is borne out by the fact that sick animals had prominent CNS signs such as paralysis, gait and balance abnormalities. In the case of human infection, the CNS symptoms and signs were very prominent and the CNS was also the most severely affected organ (Gooh e al. N Engl J. Med. 2000. 342:1229-1235; Wong et al; Am J Path. 2002. 161:2153-2167).

In the hamster kidney the vasculitis and glomerular lesions resembled those reported in humans (Chua et al. Lancet 1999. 354:1257-1259; Wong et al; Am J Path. 2002. 161:2153-2167). The consistent presence of viral antigen but not of viral genome in the covering epithelium of the renal papilla suggests possible reabsorption of IHC-detectable viral proteins leaked into the urine. Williamson et al., found evidence of urothelial infection in the urinary bladder of Hendra virus-infected guinea pigs but there was no information on epithelial infection in the kidney. The presence of viral antigen and genome in the periarteriolar lymphoid cells of the spleen suggests that active viral replication occurred there. In the hamster heart the rare infarction is assumed to be related to vasculitis as in the case of humans (Wong et al; Am J Path. 2002. 161:2153-2167)

The limited published data on NiV-infected animals comprising observations on field and experimentally-infected pigs and cats, and field-infected dogs and horse, showed that systemic vasculitis was the common feature in all these animal (Hooper et al. Microbes Infect. 2001. 3:315-322). However it appears that in none of these animals was encephalitis and neuronal infection as convincingly demonstrated, as in the hamsters in our study. In the case of the pig and cat, there was evidence of meningitis but no distinct encephalitis nor any apparent direct evidence of neuronal infection. In the dog and horse apart from meningitis, focal brain parenchyma rarefaction was also found but there is no data on the presence, if any, of encephalitis or of direct neuronal infection. Thus, these animals appear not to be good models for the acute human disease, which is typified by prominent vasculitis, encephalitis and direct neuronal infection.

Tissue localization of virus by IHC and ISH was confirmed by virus isolation and/or RT-PCR in all the solid organs tested. Overall, RT-PCR was more sensitive than virus isolation as a confirmatory test for NiV infection in both IN and IP-infected animals. The lower rate of virus isolation from IN-infected compared with IP-infected animals could be related to the longer survival of the former, which presumably favoured effective immune clearance of virus from solid organs. However, RT-PCR was negative in serum in all 7 animals tested irrespective of survival duration suggesting that the immune system may be more efficient in clearing virus from the circulation or that viremia occurred early in the infection. Alternatively, viral particles may be transported inside infected blood leucocytes. Further studies in the hamster model will be needed to clarify this.

In previous human studies viremia was also postulated to have occurred early based on the simultaneous involvement of multiple organs and disseminated blood vessels, and the observation that vascular lesions such as vasculitis, thrombosis and infarction occurred earlier than extravascular parenchymal lesions (Wong et al; Am J Path. 2002. 161:2153-2167). These findings appear to be corroborated by our data which also showed simultaneous and widespread organ involvement.

The presence of virus in urine as confirmed by RT-PCR and virus isolation correlates well with kidney glomerular injury. Virus excretion in human urine has been reported from patients and postulated as a possible means of viral transmission to health care workers.

Oral ingestion and/or aerosol inhalation of infected secretions is thought to be responsible for pig-to-human viral transmission (Parashar et al. J Infect Dis 2000. 181: 1755-1759). The successful infection of hamsters by the IN route appear to support this.

The establishment of an animal model for acute NiV infection should open the way to a greater understanding of its pathogenesis particularly in relation to the early events since present knowledge of NiV is based mainly on the end-stage disease. Potential anti-NiV drugs and vaccines could also be tested for effectiveness in the model. A greater understanding of the immune response could enable us to investigate if NiV could cause immunosupression, a phenomenon well known in measles infection. An animal model for relapsed NiV encephalitis is still elusive but long term follow-up of large numbers of infected hamsters which eventually recovered could yield some cases of relapsed encephalitis since the prevalence of human relapsed encephalitis is low (Tan et al. Ann Neurol. 2002. 51:703-708)

### Example 2

### Specific and Sensitive Quantitative Assay for Henipavirus RNA Using Real Time PCR

Nipah virus is classified as a class 4 agent and all tests have been carried out in the Biosafety level (BSL) 4 Jean Merieux laboratory in Lyon. Only RNA extracts have been tested outside the BSL4 laboratory according to biosafety procedures.

Cells and Viruses

Nipah virus (isolated from the cerebrospinal fluid of a patient) was a generous gift from Dr Kaw Bing Chua and Pr Sai Kit Lam (Kuala Lumpur, Malaysia). Virus stock was prepared in the BSL-4 laboratory by infecting Vero-E6 cells with a multiplicity of infection (MOI) of 0.01 plaque forming units (pfu)/cell and virus was recovered 24 h post-infection. The virus titer was 2×10⁷ pfu/ml.

A time-course of virus production was monitored on Vero cells infected with Nipah virus at a MOI of 0.01. Wells of subconfluent cells in Lab-tek culture plate (Nalge Nunc International) were infected with Nipah virus or mock-infected. After 1 h of incubation at 37° C., cells were washed three times with Dulbeco's minimum essential medium (DMEM) and 0.5 ml of DMEM containing 2% fetal calf serum (FCS) were added to each well. The supernatants of each well were harvested daily during four days, transferred into Eppendorf tubes, centrifuged at 2000 rpm for 5 min and then aliquoted into two fresh tubes. One series of tubes containing supernatants of infected or mock infected cells was treated for RNA extraction and quantification and the other used for virus titration.

Cell monolayers in each well were fixed in 10% formalin for 20 min and in 0.1% Triton X100 for 5 min. The cells were rinsed with PBS and incubated for 30 min at 37° with a dilution of human convalescent serum containing anti-Nipah antibodies. The cells were then rinsed and incubated with a fluorescein-conjugated anti-human IgG antibody containing a solution of 0.1% propidium iodide. After a final rinse the cells were observed in a UV microscope (Leica).

Animals

Five 7 to 14 week-old golden hamsters (Janvier, Le Fenest St Isles, France) were infected intraperitoneally with 5×10^{4 pfu} (about 100×the LD50) (Wong, et al 2003. Am. J. Pathol.). Blood samples were taken from each animal at day 5 after infection by eye puncture and the sera were frozen at −80° C. until use. We followed the French regulations for handling animals, and the procedures imposed for work in the BSL4 containment.

Virus Titration

Viruses were titrated by plaque assay on Vero cells. Briefly, six-well plates containing subconfluent Vero cells were incubated for 1 hr at 37° C. in a 5% CO₂ incubator with 1 ml of serial dilutions of virus stocks using 1:10 as the starting dilution (1:100 for hamster sera). Cells were washed twice with DMEM without FCS and covered with 2 ml of 1.6% carboxymethylcellulose in DMEM containing 5% FCS. After 5 days of incubation at 37° C., cells were fixed in 10% formalin, stained with methylene blue and rinsed with water. Plaques were counted and the titer expressed as pfu/ml.

RNA Extraction

Viral RNA was extracted from 140 μl of supernatant from Nipah virus-infected Vero cells or from 20 μl of hamster serum using the RNA extraction kit (QIAamp Viral RNA Mini Kit, Qiagen Inc., Valencia Calif., USA) following the manufacturer's instructions. The extracts were resuspended in 60 μl of Buffer AVE, aliquoted and stored at −80° C. before RT-PCR amplification was carried out.

Preparation of Positive Nipah Virus Control

The entire Nipah NP gene was cloned into the PCR TA cloning vector pDrive (Qiagen) which possesses a T7 promoter. The sequence and orientation of the insert were verified by DNA sequencing (Big Dye Terminator, Applied Biosystems, USA). The plasmid pDrive-NP-NiV was linearized at the end of the NP gene and then purified using the Geneclean® II kit (Q-Biogene) prior to in vitro transcription using T7 RNA polymerase (Invitrogen). The RNA transcripts were treated with RNase-free DNase I (Roche diagnostics) to remove the DNA template, and then extracted with RNA NOW (Ozyme) and ethanol precipitated. The RNA was resuspended in water and stored at −80° C. To ensure that template DNA had been eliminated, a quantitative PCR assay was performed using the TaqMan™ PCR system (TaqMan™ universal PCR Master Mix 200RXN, Applied Biosystems) before and after the treatment with RNase-free DNase I. The amount of RNA was determined by spectrophotometer and measured quantities were used to realize the standard curve for Real time RNA quantification.

Primers and TaqMan™ Probes

The primers and probe for the Nipah NP gene were designed using the program Primer Express™ (Perkin-Elmer, Applied Biosystems, USA) following the recommended criteria. The forward primer (Ni-NP1209 5′GCAAGAGAGTAATGTTCAGGCTAGAG 3′—SEQ ID NO:1) and the reverse primer (Ni-NP1314 5′ CTGTTCTATAGGTTCTTCCCCTTCAT 3′—SEQ ID NO:2) amplify a 105 bp fragment. The fluorescent probe (Ni-NP1248Fam 5′ TGCAGGAGGTGTGCTCATTGGAGG 3′—SEQ ID NO:3) was designed to anneal to a sequence internal to the PCR primers. The fluorescent reporter dye, a 6-carboxy-fluorescent (FAM) was located at the 5′ end of the probe and the quencher 6-carboxy-tetramethyl-rhodamine (TAMRA) was located at the 3′ end.

RT-PCR TaqMan™ Reaction

Quantitative RT-PCR assays were performed using the ABI PRISM 7700 TaqMan™ sequence detector. The one-step RT-PCR system (TaqMan™ one step PCR master Mix reagents kit, Applied Biosystems) was used for an uninterrupted thermal cycling. A master mix reaction was prepared and dispensed in 20 μl aliquots or 22.5 μl aliquots into thin-walled microAmp optical tubes (ABI PRISM™, Applied Biosystems). Then 5 μl of RNA extract from hamster sera, or 2.5 μl from either stock virus or infected cell supernatants, or 2.5 μl of RNA transcript were added to each tube. The final reaction mixture contained 900 nM of each primer and 200 nM of the probe. Prior to amplification the RNA was reverse transcribed at 50° C. for 30 min. This was followed by one cycle of denaturation at 94° C. for 5 min. Next, PCR amplification was carried out for 45 cycles at 94° C. for 15 s and 60° C. for 1 min. The fluorescence was read at the end of this second step allowing a continuous monitoring of the amount of RNA. The threshold cycle (Ct) is the number of cycles before the fluorescence emitted passed a fixed limit called the ‘detection threshold’ (Dt). The determination of the Dt was based on the lowest level at which viral RNA was detected and remained within the range of linearity of a standard curve. Thus, the log₁₀ of the number of targets initially present is proportional to the Ct value and can be measured using the standard curve.

RNA from the measles virus strain CR68, whose quality had been verified, was used as a negative control.

These experiments show an assay to detect and quantify Nipah virus RNA that is versatile, highly reproducible and stable over time. To achieve this we have developed a Nipah virus TaqMan™ RT-PCR assay.

Sensitivity and Specificity of the Assay

The sensitivity and specificity of the Nipah virus detection assay were evaluated by using a series of samples containing dilutions of RNA extracted from a Nipah virus stock. A range of 10 fold virus dilutions containing from 1.2×10⁵ pfu to 0.12 pfu per tube (in a volume of 2.5 μl) was tested. A threshold cycle (Ct) value was calculated from the amplification plot of this range of dilutions (FIG. 1). FIG. 2 shows that the detection was linear from 1.2×10⁵ pfu to 1.2 pfu per run. This indicates both the feasibility of the amplification test for a large range of virus titers and its sensitivity. Similar data were obtained when the test was repeated three times, underlining the reproducibility of the assay (data not shown). The specificity of the assay was verified by the absence of amplification using measles virus RNA with Nipah primers and probe (data not shown).

To standardize the assay, serial dilutions of known amounts of RNA transcribed in vitro from the plasmid pDrive-NP-NiV were tested by RT-PCR TaqMan™. Three assays using transcript RNAs prepared at different days were used to draw a standard curve (FIG. 3). The linearity of the curve allowed a quantification of 10⁹ to 10³ molecules of RNA per reaction. Moreover, the low deviation (R2=0.9834) indicates that the assay is highly reproducible (FIG. 3). The inter-assay coefficient of variation calculated by comparing the Ct values obtained for two RNA transcripts was found to vary between 0.3 to 2.2%.

Quantification of Virus Load in Infected Cell Supernatants

To determine the accuracy of our TaqMan™ RT-PCR method for quantification of Nipah virus RNA, infectious virus titers obtained by plaque assays were compared to the amounts of genome equivalents calculated by TaqMan™ RT-PCR using a RNA transcript standard curve. Vero cells were infected with Nipah virus at a multiplicity of infection of 0.01 pfu/cell and cell supernatants taken at days 1, 2, 3 and 4 post infection were analysed. A mild virus-induced cytopathic effect was already observed one day post-infection, and the number and intensity of cell fusions increased each day until full cell destruction was complete 4 days post-infection (FIG. 4). The amounts of infectious virus and viral RNA in the medium increased until the third day for each infection, and then decreased (FIG. 5). Moreover, the RNA/pfu ratios between the number of infecting particles and the number of RNA genomes were not constant, and increased with the time of infection (Table 1).

TABLE 1
Detection of infectious Nipah virus and Nipah
virus RNA in infected cell supernatants by plaque
assays and real-time RT-PCR assay.

| | Viral RNA/ml | | pfu/ml | | | |
|---|---|---|---|---|---|---|
| Days | (×10⁻⁶)^{b} | | (×10⁻³) | | RNA/pfu^{c} | |
| post | No Test | | | | | |
| infection ^{a} | 1 | 2 | 1 | 2 | 1 | 2 |
| 1 | 11 | 5 | 21 | 9 | 538 | 507 |
| 2 | 760 | 2007 | 775 | 2037 | 981 | 1049 |
| 3 | 1924 | 3353 | 1210 | 2275 | 1590 | 1473 |
| 4 | 1549 | NT* | 400 | NT | 3872 | NT |

^{a} Cells were infected at MOI of 0.01 and supernatants were analysed at 1, 2, 3 and 4 days after infection.
^{b} The concentration of Nipah virus RNA was calculated using the RNA transcript standard curve.
^{c} RNA/pfu ratios between the number of infecting particles and the number of viral RNA detected in Vero cell supernatants.
*Not tested

To confirm the accuracy of the number of viral RNA molecules in a sample, ten fold dilutions of RNA extracted at day 3 post-infection were analysed by TaqMan™ and compared to the theoretical number of pfu (Table 2). Day 3 was chosen because it corresponded to the peak of RNA and infectious virus production. The RNA/pfu ratios obtained in diluted samples at day 3 after infection increased inversely to the amount of viral RNA.

TABLE 2
Quantification of Nipah RNA from diluted supernatants of cells infected by 0.01 pfu/cell
3 days after infection by real time RT-PCR TaqMan ™ and RT-PCR.

| Test 1 | | | | Test 2 | | |
|---|---|---|---|---|---|---|
| pfu/ml (×10⁻³) | RNA/ml (×10⁶) | RNA/pfu | RT-PCR | pfu/ml (×10⁻³) | RNA/ml (×10⁶) | RNA/pfu |
| 1210 | 1738 | 1436 | + | 2275 | 3090 | 1358 |
| | (1924)^{a} | (1590) | | | (3353)^{a} | (1473) |
| 121 | 229 | 1891 | + | 227.5 | 308 | 1353 |
| 12.1 | 31 | 2543 | + | 22.75 | 36.9 | 1622 |
| 1.21 | 4 | 3349 | + | 2.275 | 3.9 | 1714 |
| | | | | 0.2275 | 0.59 | 2588 |
| | | | | 0.0228 | UD^{b} | |

^{a}The value in parentheses was calculated in the experiment described in Table 1
^{b}Unquantifiable data (RNA was detected in the sample but the Ct value was out of the range of linearity of the standard)

Detection of Viral RNA in Sera of Hamsters Infected with Nipah Virus

To assess whether our Nipah TaqMan™ assay allows the detection and quantification of viral RNA in biological samples, the sera of five hamsters infected with Nipah virus were analysed by plaque titration and real time RT-PCR. Previous studies have shown that viremia in hamsters could be detected at day five post infection. The results (Table 3) indicate that viral RNAs were detected in three animals and infectious virus in two animals. The number of viral genome molecules was about 3 logs higher than the number of live virus.

TABLE 3
Detection of Nipah viral RNA in sera of infected
hamsters extracted 5 days after infection.

| Hamsters | ARN/ml (10⁻³) | Pfu/ml | RNA/pfu |
|---|---|---|---|
| H1 | 705 | 500 | 1410 |
| H2 | 1413 | 500 | 826 |
| H3 | 628 | ND | |
| H4 | ND^{a} | ND | |
| H5 | ND | ND | |

^{a}not detected
Hamsters were infected intraperitoneally with 100 times the dose needed to kill 50% of the animals. The quantification of the amplification plot was calculated with a curve using RNA transcripts

The assay that has been developed provides a rapid, accurate and quantitative diagnosis of Nipah virus infection. This test can be a useful tool for laboratories that need to rapidly confirm the etiology of Nipah virus in clinical or field specimens. Nipah virus is highly pathogenic for man and has killed more than 40% of infected individuals (Goh, et al 2000, New Engl J. Med. 342:1229-35; Chong, et al 2002, Can J Neurol Sci. 29:83-7; Lee, et al 1999, Ann Neurol. 46:428-32). In pigs, mortality is low but because the infection rate approaches 100%, to stop the spread of Nipah virus, over one million pigs were slaughtered in Malaysia in 1999, which had a devastating impact on the national pig farming industry (Mohd Nor et al 2000, Rev Sci Tech Off Int Epiz. 19(1):160-5; Chua, 2000, Science. 288:1432-5). Although no human or pig cases have been identified since the last epidemics in Malaysia and in Singapore, the presence of pteroid bats carying anti-Nipah antibodies in Cambodia in 2001 indicates that the virus may reemerge at any time in southeast Asia. A Nipah-like disease was reported in Bangladesh in 2001 and in Northen India, but as yet no precise data concerning the nature of the etiolologic agent has become available (ProMed 2002 Nipah-like virus—Bangladesh (2001): Archive number 20020830.5187; ProMed 2003 Nipah-like virus—India (North Bengal):2001 Archive number 20030106.005027). A positive identification of this virus is necessary to implement appropriate control measures. However, the absence of therapy or a vaccine against this agent imposes that its propagation in cell culture for virus isolation and identification, serum neutralization, and antigen preparation for ELISA, be conducted in a biosafety level BSL 4 laboratory. Such restrictions would limit both investigations of encephalitis in humans, and virus detection in biological specimens of wild and domestic animals. To ensure operator safety, the use of diagnostic real-time PCR assays for Nipah virus should be a prerequisite safe approach for preliminary identification of specimens that can then be handled in a BSL-4 laboratory for propagation.

TaqMan™ assays have been developed to diagnose a large range of viruses such as varicella zoster, human papilloma, hepatitis C, dengue, Epstein-Barr, or influenza viruses (Hawrami, et al 1999, J Virol Methods. 79:33-40; Josefsson, et al 1999, J Clin Microbiol. 37:490-496; Morris, et al 1996, J Clin Microbiol. 34:2933-2936; Laue, et al 1999, J Clin Microbiol. 37:2543-2547; Leung, et al 2002, J Immu Methods. 270:259-267; Schweiger, et al 2000, J Clin Microbiol. 38: 1552-1558) and the technique has been used to assist in the diagnosis of several life-threatening enzootic mosquito-borne and hemorrhagic viral diseases (Lanciotti, et al 2000, J Clin Microbiol. 38:4066-4071; Garin, 2001, Microbes Infect. 3:739-745; Garcia, et al 2001, J Clin Microbiol. 39:4456-4461; Houng, et al 2000, J. Virol. 86:1-11). Real-time RT-PCR has the advantage over plaque assays and RT-PCR in that it provides rapid, quantitative and specific results.

The TaqMan™ assay developed for Nipah virus detected a wide range of virus concentrations from 1.2×10⁵ pfu to 1.2 pfu per reaction, corresponding to a threshold of 200 pfu/ml. Other studies on differents viruses have shown similar detection threshold (Houng, et al 2000, J. Virol. 86:1-11; Lanciotti, et al 2000, J Clin Microbiol. 38:4066-4071). The sensitivity of the Nipah TaqMan™ assay was found to be similar to those obtained with RT-PCR (Table 2).

The reproducibility of the TaqMan™ assay was high since only small variations were observed in the results from several assays conducted at different times and with different RNA preparations (see FIG. 3 and Table 2). Thus the reliability of the test may principally depend on RNA extraction. The specificity of the Nipah virus TaqMan™ assay was verified by the absence of measles virus RNA amplification when the Nipah virus-specific primers and probe were used. Measles virus is a morbilivirus, the most closely related genus to henipaviruses. A TaqMan™ assay has recently been developed for Hendra virus, a henipavirus showing 78.4% nucleotide homology in the N gene with Nipah virus (Smith, et al 2001, J Virol Methods. 98:33-40; Wang, et al 2001, Microbes and Infection 3, 279-287). The analysis by the program Primer Express of the affinities of the Nipah virus probe, and the forward and reverse primers for the Hendra virus N gene suggests that the test should be specific for Nipah virus (Harcourt, et al 2000, Virology. 271:334-349). The specificity of the Nipah virus TaqMan assay in the Henipavirus genus was verified with Hendra virus. The absence of Hendra virus RNA amplification with the Nipah virus-specific primers and probe confirms the specificity of the test for Nipah virus.

RNA transcripts were developed as stable, reproducible and reliable standards for quantitative assays. The linear range of Nipah virus RNA quantification was at least 10⁹ to 10³. Similar results were obtained for Hendra virus: the linearity was observed from undiluted Hendra virus RNA to 1/10⁷ (Smith, et al 2001, J Virol Methods. 98:33-40). This range of linearity allows the detection of a wide range of virus titers and should quantitatively identify Nipah virus in clinical specimens and in cell cultures without requiring dilutions of the sample. Surprisingly, the ratio of RNA molecules/pfu increased when the virus was diluted in the test tube (Table 2), suggesting that high quantities of RNA molecules may affect the efficiency of DNA amplification. This may be explained by the lack of reagents available in the samples containing high quantities of RNA templates.

The number of viral genome molecules calculated by TaqMan™ assay was found to be about 3 logs higher than the corresponding number of infectious virus particles measured by plaque titration. For dengue virus, it was also found that each infectious pfu contained at least 100 or more genomic equivalents and for Rift Valley Fever or Puumala virus a 2-3 log difference was noted (Houng, et al 2000, J. Virol. 86:1-11; Garcia, et al 2001, J Clin Microbiol. 39:4456-4461; Garin, 2001, Microbes Infect. 3:739-745). This ratio is due to the presence of non-infectious virus, either to defective, immature, or inactivated particles, or to RNA encapsidated as nucleoparticles released from damaged infected cells. Indeed, the RNA/pfu ratios calculated at different times after infection increased with the time of infection, with the highest ratio observed at day 4, mirroring the cytopathic effect (FIG. 4).

These data show that the Nipah TaqMan™ RT-PCR assay is also valid for monitoring Nipah virus in serum samples from infected hamsters. Sera were taken at day 5 post-infection because this was the only day when virus could ever be detected in animals (V. Guillaume et al., J. Virol. 2004. 78: 834-840). However, both real-time PCR and plaque titration failed to demonstrate Nipah virus in two out of five hamsters, confirming that these animals may have suffered either a brief or an undetectable viremia. Viral RNA but not virus was detected in hamster H3. However, virus titers in the hamster sera were rather low and close to the limits of detection of both techniques (200 pfu/ml and 100 pfu/ml for real-time RT-PCR and plaque titration, respectively).

### Example 3

### Vaccination and Passive Protection Against a Henipavirus

In the following, two NiV glycoproteins (G and F) in vaccinia virus recombinants have been expressed to evaluate their contribution to protection. To do this a hamster animal model in which the animals die of acute encephalitis following Nipah virus infection was used and presented as example 1 (Wong et al. Am. J. Patol. 2003. 163:2127-2137) Using this model, vaccination with vaccinia recombinants expressing either of the two Nipah virus glycoproteins protects the animals from a fatal infection. Furthermore, passive transfer of antibody from immunized animals to naive animals protects the latter from a lethal Nipah virus challenge.

Cells and Viruses

Vero E6, RK13 and BHK 21 cells were maintained in DMEM medium (Gibco) containing 10% foetal calf serum. Nipah virus isolated from the cerebrospinal fluid of a patient was received at the Jean Merieux BSL-4 laboratory in Lyon, France, from Dr K B Chua and Dr S K Lam (University of Malaya, Kuala Lumpur, Malaysia) following two passages in Vero cells. A virus stock was made (under P4 conditions) following a third passage on Vero cells: the supernatant was harvested 2 days after infection when the Vero cells showed fusion and syncytia formation. The virus stock was titrated in 6-well plates by incubating 200 μl of serial 10 fold dilutions of supernatant in each well (containing 106 Vero cells per well) for 1 hr at 37° C. The cells in each well were then washed twice with DMEM and 2 ml of 1.6% carboxymethylcellulose in DMEM containing 2% fetal calf serum were added to each well. The plates were incubated for 5 days at 37° C., and the wells were washed with phosphate buffer pH 7.4 (PBS), fixed with 10% formalin for 20 min, washed and stained with methylene blue. After infecting Vero cells at a multiplicity of infection (m.o.i.) of 0.01 pfu/cell, virus titres reached 2×10⁷ pfu/ml.

Stocks of vaccinia and recombinant viruses were grown in BHK 21 cells. Cells were infected at 0.01 pfu/cell and the cells harvested 3 days later, sonicated and stored at −80° C. Virus was titrated in Vero cells.

Cloning of NiV Glycoprotein Genes and Construction of Vaccinia Recombinants

To clone the NiV genes coding for the two viral glycoproteins, Vero E6 cells infected with NiV were extracted with RNA Now according to the manufactures instructions and subjected to RT-PCR. The 5′ and 3′ primers used for the G protein were 5′-CGCGGATCCAGTCATAACAATTCAAG-3′ (SEQ ID NO:4) and 5′-CGCGGATCCGAGGTTGATTTTTATG-3′ (SEQ ID NO:5) respectively. Those for the F protein were 5′-CGCAGGATCGAAGCTCTTGCCTCG-3′ (SEQ ID NO:6) and 5′-CATCAATCTGGATCCACTATGTCCC-3′ (SEQ ID NO:7). The resulting cDNA was cloned into pT-Adv plasmid using Clontech Advantage PCR cloning kit according to the manufacture's instructions. Nucleic acid sequence analysis revealed that, compared to the published nucleic acid sequence analysis for NiV (Chan, et al 2001. J Gen Virol. 82:2151-5), there was a single nucleotide difference in the NiV.G gene at position 683 (A to G) but this change is silent as far as the primary sequence is concerned. VV recombinants were prepared using the host-range selection system described by Perkus et al. (Perkus, et al 1989. J. Virol. 63:3829-3836). Briefly, the genes to be expressed were subcloned by excising the inserts from the pT-Adv plasmids with Bam HI and cloned into the Bam HI site of the pCOPAK H6 plasmid (Perkus, et al 1989. J. Virol. 63:3829-3836), which also contains the KIL vaccinia gene. Vero cells were infected with the NYVAC strain of VV (Tartaglia et al 1992. Virology. 188:217-232) and transfected with the pCOPAK plasmid. The VV recombinants were selected on RK13 cells.

Antibody Determinations

Sera from hamsters were tested individually by enzyme-linked immunosorbent assay (ELISA) for the presence of NiV antibodies. Crude extracts of NiV antigens were prepared from Vero cells infected at a m.o.i. of 0.01 pfu/cell for 24 hours. The cells were washed with PBS and lysed in PBS containing 1% Triton X100 (10⁷ cells/ml) at 4° C. for 10 min. The cell lysate was sonicated twice for 30 seconds each to full cell destruction and centrifuged at 5000 rpm at 4° C. for 10 min. The supernatant was frozen at −80° C. Non-infected Vero cells were similarly treated to prepare control antigen. Cross-titration of the Nipah antigens was performed with serum from a convalescent, NiV-infected patient to determine the antigen titer corresponding to the dilution showing the highest O.D. reading.

Neutralizing antibody titres were determined in Vero cells. Serum dilutions in PBS starting with 1/20 were mixed with 50 pfu of NiV in 96 well plates and incubated for 1 hour at 37° C. and then 20,000 Vero cells were added. The plates were read after 5 days and the dilution of serum reducing 50% of the virus titre was recorded.

Primers and TagMan™ Probes

The conditions used are those described above in Example 2. Briefly, the primers and probe were designed using the program Primer Express™ (Perkin-Elmer, Applied Biosystems, USA) following the recommended criteria. A target region in the NP gene was selected. The forward primer (NiV.NP 1209 5′-GCAAGAGAGTAATGTTCAGGCTAGAG-3′ (SEQ ID NO:1)) and the reverse primer (NiV.NP1314 5′-CTGTTCTATAGGTTCTTCCCCTTCAT-3′ (SEQ ID NO:2)) amplify 105 pb of the NiV.NP gene. The fluorescent probe (NiV.NP124SFam 5′-TGCAGGAGGTGTGCTCATTGGAGG-3′ (SEQ ID NO:3)) is designed to anneal to a sequence internal to the PCR primers. The fluorescent reporter dye, 6-carboxy-fluorescein (FAM) was located at the 5′ end of the probe and the quencher, 6-carboxy-tetramethyl-rhodamine (TAMRA) was located at the 3′ end.

Quantitative RT-PCR assays were performed using the ABI PRISM 7700 TagMan sequence detector. The one-step RT-PCR system (TagMan one-step PCR master Mix reagents kit, Applied Biosystems) was used for uninterrupted thermal cycling. A master mix reaction was prepared and dispensed in 201 aliquots or 22.5 μl aliquots into thin-walled microAmp optical tubes (ABI PRRSMTM, Applied Biosystems) allowing a continuous monitoring of the amount of RNA. Then 5 μl of RNA extract from sera or 2.5 μl RNA transcript was added to each tube. The final reaction mixture contained 900 nM of each primer and 200 nM of the probe. Prior to amplification the RNA was reverse transcribed at 50° C. for 30 nm. This was followed by one cycle of denaturation at 94° C. for 5 nm. PCR amplification then proceeded with 45 cycles at 94° C. for 15 s, 60° C. for 1 mn.

Immunization of Hamsters

For protection studies, inbred golden hamsters (Janvier, Le Fenest St. Isles, France), were vaccinated twice (1 month apart) with 10⁷ pfu of VV recombinants expressing either the G or F NiV glycoproteins or with 5×10⁶ of each of the recombinants when they were used for co-immunization. The animals were challenged 3 months after the last immunization.

To prepare polyclonal monospecific serum against the F and G glycoproteins, hamsters were immunized on day 0 and 14 with 10⁷ pfu of the VV recombinants followed by sonicated VV-recombinant infected BHK 21 cells (+Freund's complete adjuvant) at 28 days and the same antigen (+Freund's incomplete adjuvant) at 42 days. The animals were bled 14 days after the last immunization and the antibodies determined by ELISA and neutralization.

Expression of NiV Glycoproteins in Vaccinia

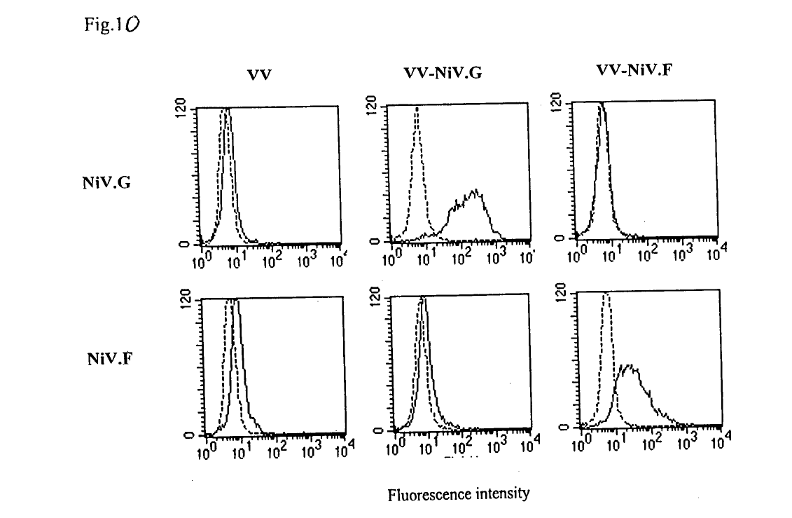

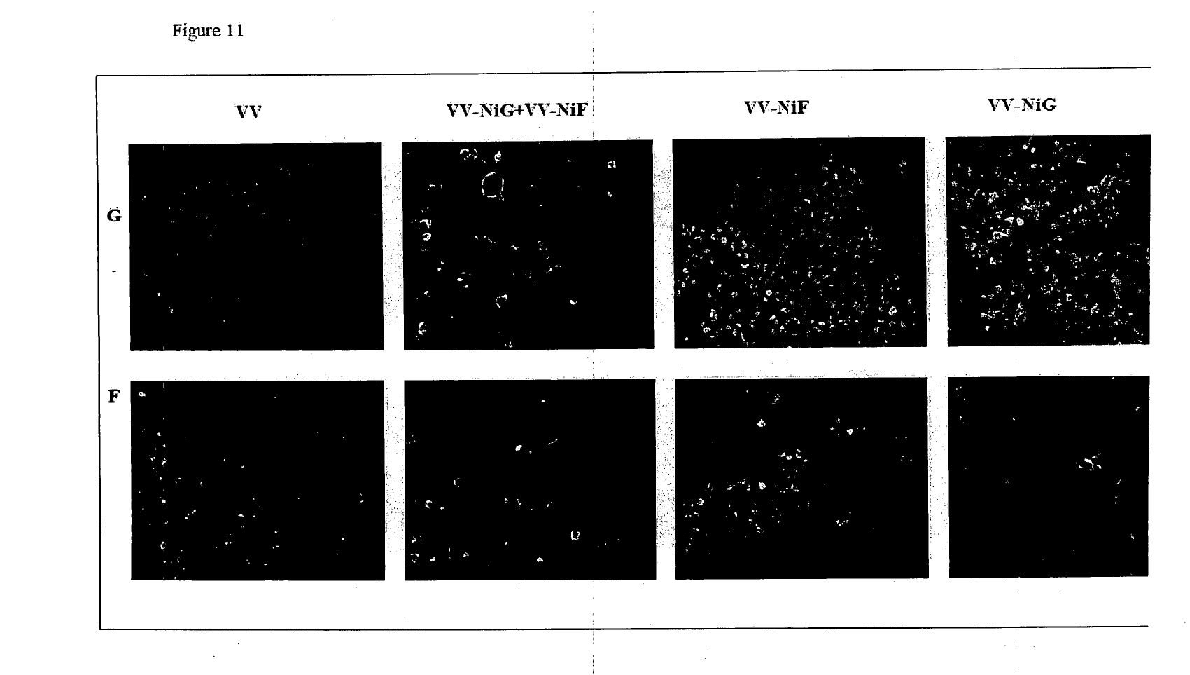

The NiV G or F proteins expressed from vaccinia virus were tested in vitro for the expression of biologically active proteins. HeLa cells infected with either VV-NiV.G or -F were examined by FACScan analysis for the expression of the NiV proteins at the plasma membrane. Both viral glycoproteins were expressed at the cell surface (FIG. 10). When HeLa cells were infected with both vaccinia recombinants cell fusion (syncytia formation) was induced (FIG. 11).

Immunization of Hamsters with VV Recombinants Expressing G or F Protects Against a Lethal Infection

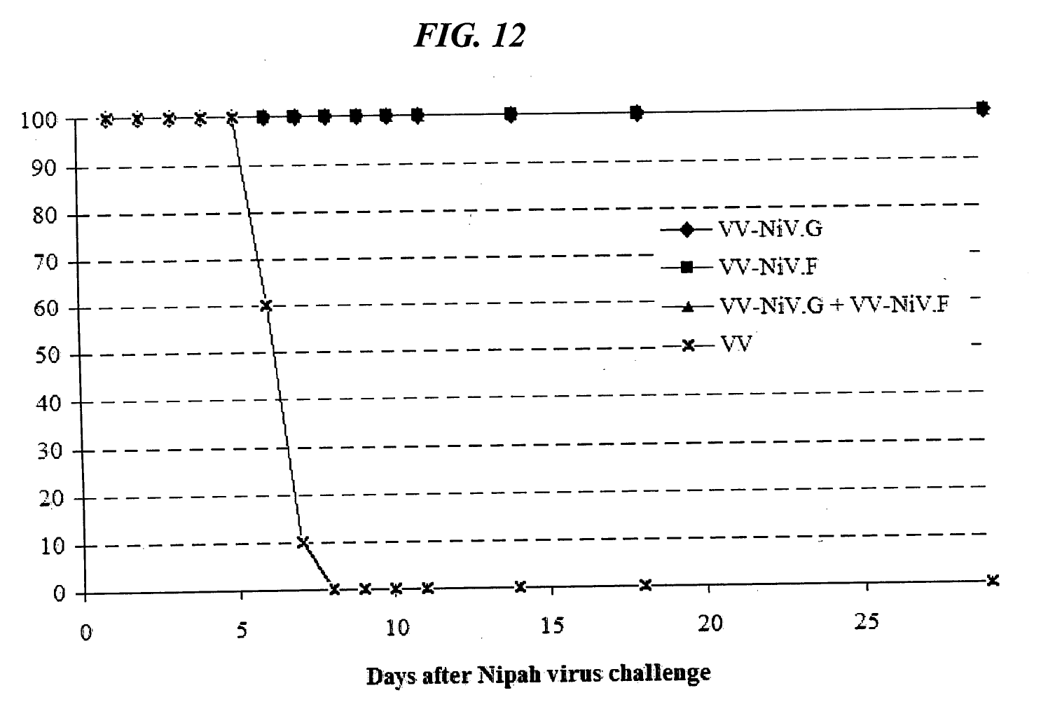

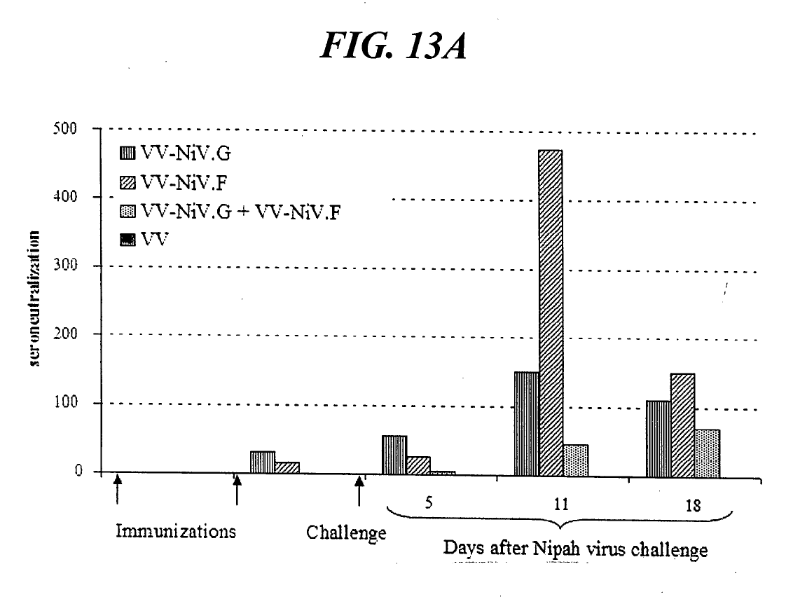

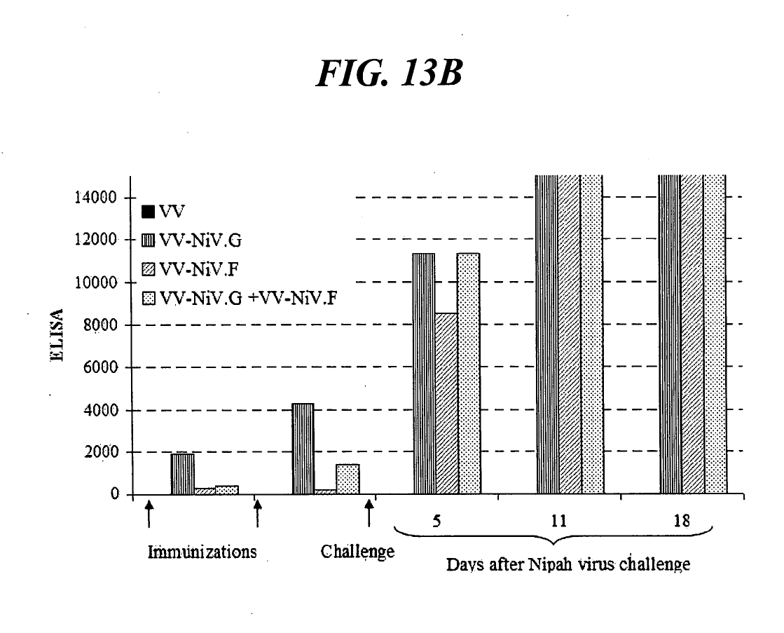

Hamsters were immunized subcutaneously with either 10⁷ pfu VV-NiV.G or F or with the two combined (5×10⁶ pfu of each recombinant). One month later, the animals were boosted with the same dose of vaccinia recombinant. In the animal model we have developed for NiV, intraperitoneal inoculation of hamsters with our NiV isolate induces a fatal encephalitis 7-10 days later (See example 1 and FIG. 1). When the VV-NiV.G, -F or G+F vaccinated animals were challenged with NiV 3 months after the last immunization, there was complete protection against mortality (FIG. 12). After challenge, the levels of both neutralizing and antibodies as measured by ELISA increased in all vaccinated animals (FIG. 13). Further studies on the sera from the hamsters showed that the presence of virus could only be detected at a late stage of infection (day 5-6) in control non-immunized animals. No virus was detected in the vaccinated animals (Table 4).

TABLE 4
quantitative analysis of NiV present in the
sera in control and infected hamsters

| | Number of hamsters with Nipah virus RNA | | | |
|---|---|---|---|---|
| | detected by TaqMan assay* | | | |
| | VV-NipG | VV-NipF | VV-NipG/VV-NipF | control |
| J1 | — (4) | — | — | — |
| J2 | — | — | — | — |
| J3 | — (4) | — | — | — |
| J4 | — | — | — | — |
| J5 | — (4) | — | — | 4 (5) |
| J6 | — | — | — | 2 (3) |
| J7 | — (4) | — | — | |
| J8 | — | — | — | |

*five animals were tested each day for each vaccination test

Serum from VV-NiV.G and -F recombinant-immunized hamsters passively protects naive hamsters against a lethal NiV challenge.

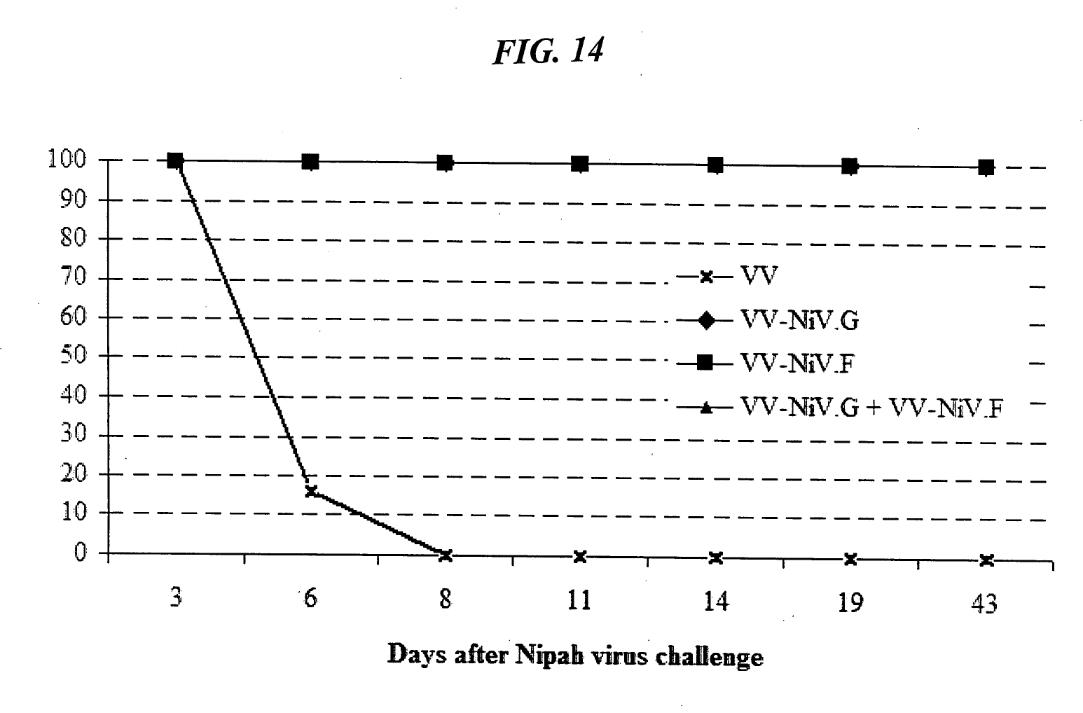

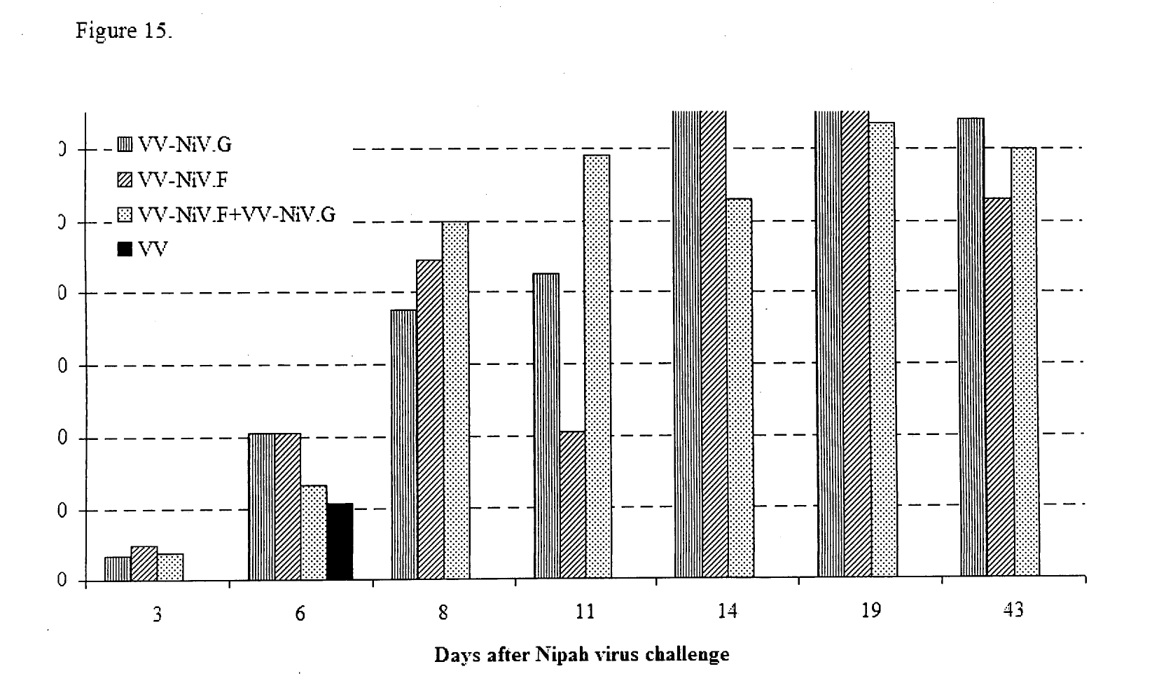

To dissect the importance of the humoral immune response in protection, hamsters were hyperimmunized with the vaccinia recombinants (see Materials & Methods) and the animals with sera containing the highest levels of neutralizing antibody to NiV were pooled (160 neutralizing units/ml). Hamsters were given 0.2 ml of anti-serum directed against either the G or F NiV glycoproteins or a mixture of the two by intraperitoneal injection. One hour later the animals were challenged with virus and 24 hr later 0.2 ml of sera were again passively transferred. The hamsters were observed for clinical signs during two months. Animals receiving either of the anti-sera (monospecific polyclonal G or F) or the mixture of the two were protected from a lethal NiV infection (FIG. 14). After infection the ELISA serum antibody levels against NiV were strongly induced (FIG. 15).

The above shows the immunological parameters which may play a role in protection against NiV infection.

Hamsters vaccinated with either VV.G or F were completely protected from a lethal infection. Confirming the contribution of the humoral response in this process, naive animals were also shown to be protected by hyperimmune serum passively transferred prior to challenge. Thus, using an animal model the above shows that it is possible to protect both actively and passively against lethal NiV infections. However, in both active and passive immunization the antibody response to NiV was strongly stimulated, suggesting that the virus replicated in the vaccinated animals. However, attempts to detect virus in the sera were unsuccessful. In control non immunized animals, virus could only be detected in the sera of moribund animals. It is probable, as observed in several other paramyxovirus infections, that the virus is mainly cell-associated.

In humans, both relapsing and late onset cases of infection have been observed (Lim, et al 2003. J. Neurol. Neurosurg. Psychiatry. 74:131-133; Tan, et al 2002. Ann Neurol. 51:703-708; Wong, et al 2001. J Neurol Neurosurg Psychiatry. 71:552-554). In these situations the immunobiology of the infection is unknown. These late pathologies in our challenged immunized animals up to 5 months post-challenge have not been observed. Similarly, in the passively protected animals no late disease was observed. However, the lower limits of antibody protection in vivo or the effect of passively immunizing the animals once the infection has been initiated have not been determined.

Obviously, numerous modifications and variations of the present invention are possible in light of the above teachings. It is therefore to be understood that within the scope of the appended claims, the invention may be practiced otherwise than as specifically described herein.

### Example 4

### Production and Reactivities of Monoclonal Antibodies Against Nipah Virus

In order to study the pathology of Nipah virus infections, we have established a hamster model (part of the claim). Following infection with Nipah virus, the animals die from encephalitis displaying a pathology similar to that seen in man. Furthermore, we have shown that these animals can be protected either by vaccination using either of the glycoproteins (G or F) or passively using antisera directed against one of these antigens (part of the claim). As there is, as yet, no treatment available for Henipavirus infections, we will develop an immunotherapeutic approach to develop prophylactics for Henipavirus-infected individuals. We have developed a bank of monoclonal antibodies (mAbs) against the NiV G and F glycoproteins and which neutralise Nipah virus infectivity in vitro. Furthermore, certain of the anti-NiVF mAbs neutralise Hendra virus.

Present Situation and Materials Available

We have characterised 30 mAbs from a bank prepared against G- or F-expressed Nipah virus proteins. -17 against NiF and 13 against NiG. On the basis of virus neutralisation, certain have been selected for the present study. It should be noted that none of the anti-NiGs neutralised Hendra virus, whereas the anti-NiFs also neutralised HeV. The epitopes recognised by these NiV mAbs have been studied by competition ELISAs and also by sequencing escape mutants. The properties of the mAbs selected for the initial studies are shown below:

| | | | a.a. | | |
|---|---|---|---|---|---|
| | | | recognised | | |
| | | | (escape | neutralisation specificity | |
| Antigen | name | isotype | mutants) | NiV | HeV |
| G | 1.7 | IgG1 | 336,391 | 1.7 × 10⁶ | — |
| G | 3B10 | IgG1 | 500,533 | 0.5 × 10⁶ | — |
| G | 5A7 | IgG2a | n.d. | 1.1 × 10⁵ | — |
| G | 7F3 | IgG2b | n.d. | 1.3 × 10⁵ | — |
| F | 35 | IgG1 | 282(NiV), | 3.5 × 10⁵ | 3.5 × 10⁵ |
| | | | 216(HeV) | | |
| F | 3 | IgG2a | 247(NiV & | 2.4 × 10⁵ | 1.2 × 10⁵ |
| | | | HeV) | | |

For analyses of the immune responses after NiV infection, we have expressed the G, F and NP NiV proteins in vaccina virus. These antigens obtained from infected cell lysates are used in ELISA tests to measure antigen specific responses.

Balb/c mice have been immunised with the expression plasmid VIJ containing the cDNA of the Nipah virus G or F protein. This has been performed using the gene gun (BioRad) technique. The mice have been boosted with a vaccinia recombinant encoding the Nipah virus G or F protein and 3-4 months after this boost, the mice have been injected (i.p.) with irradiated Nipah vius-infected Vero cells 3 days prior to the fusion. The hybridomas have been screened for IgG secreting hybridomas on Nipah virus-infected and non-infected Vero cells.

We have characterised all the NiV mAbs by neutralisation and a number by competition ELISA and sequence analysis of escape mutants. Overall, our studies so far indicate that there is probably a single major epitope in F or in G protein recognised and the data from the escape mutants suggest that the different mAbs overlap the region to varying degrees.

1. An golden hamster animal model of Henipavirus infection, which is infected with a Henipavirus.

2. A method of detecting Nipah virus in a sample, comprising:
producing a DNA copy of at least one RNA molecule of said Nipah virus with at least one primer specific for the RNA molecule;
amplifying the DNA copy with at least one pair of oligonucleotide primers specific for the DNA copy of the Nipah virus RNA molecule; and
detecting the presence of an amplified DNA corresponding to Nipah virus, which is indicative of the presence of Nipah virus in the sample.

3. The method of claim 2, wherein the DNA copy produced and amplified is a Nipah virus nucleocapsid coding region.

4. The method of claim 2, wherein at least one of the pair of oligonucleotide primers comprises a detectable moiety.

5. The method of claim 4, wherein the detecting comprises visualizing the detectable moiety.

6. The method of claim 2, wherein the sample is obtained from a pig.

7. The method of claim 2, wherein the sample is obtained from a wild or domestic animal

8. The method of claim 2, wherein the sample is obtained from a human.

9. The method of claim 2, wherein the at least one primer specific for the RNA molecule comprises at least 15 consecutive nucleotides of complementary to a polynucleotide which encodes a polypeptide comprising an amino acid sequence selected from the group consisting of SEQ ID NO:16, SEQ ID NO:23, SEQ ID NO:31 and SEQ ID NO:32.

10. The method of claim 9, wherein the at least one primer specific for the RNA molecule comprises at least 20 consecutive nucleotides of the polynucleotide.

11. The method of claim 9, wherein the at least one primer specific for the RNA molecule comprises at least 25 consecutive of the polynucleotide.

12. A method of protecting an individual against a Henipavirus infection comprising:
administering the at least one isolated Henipavirus G and F glycoproteins to said individual or mammal in an amount sufficient to induce an immune response in said individual or mammal.

13. The method of claim 12 wherein the Henipavirus is Nipah or Hendra virus.

14. The method of claim 12, wherein said administering further comprises administering an adjuvant.

15. The method of claim 12, wherein said administering is performed one or more times.

16. The method of claim 15, wherein at least both the Henipavirus F and G glycoproteins are administered.

17. A method of preventing or protecting an individual or mammal in need thereof against Henipavirus infection comprising:
administering an expression vector, which expresses at least one isolated Henipavirus G and F glycoproteins to said individual or mammal in an amount sufficient to induce an immune response in said individual or mammal to prevent or protect the individual or mammal against Henipavirus infection.

18. The method of claim 17, wherein the expression vector expresses at least F and G glycoproteins of the Henipavirus.

19. The method of claim 17, wherein the expression vector is a viral vector.

20. The method of claim 19, wherein the viral vector is a recombinant poxvirus vector.

21. The method of claim 17, further comprising administering at least one adjuvant.

22. A recombinant hybridoma which produces an antibody against one or both of a Henipavirus G or F protein.

23. A recombinant poxvirus vector expressing one or both of a Henipavirus G or F protein.

24. A recombinant vaccinia virus expressing Nipah G protein deposited at the CNCM as No. I-3086.

25. A recombinant vaccinia virus expressing Nipah F protein deposited at the CNCM as No. I-3085.

26. Hybridoma N°1.7 anti-Nipah virus G protein with neutralizing activity against Nipa virus.

27. Hybridoma N°3.B10 anti-Nipah virus G protein with neutralizing activity against Nipa virus.

28. Hybridoma N°35 anti-Nipah virus F protein with neutralizing activity against Nipah and Hendra virus.

29. Hybridoma N°3 anti-Nipah virus F protein with neutralizing activity against Nipah and Hendra virus.


SEQUENCE LISTING (45 sequences)

SEQ ID NO: 1 - DNA, 26 bp, Artificial Sequence
  synthetic oligonucleotide
gcaagagagtaatgttcaggctagag

SEQ ID NO: 2 - DNA, 26 bp, Artificial Sequence
  synthetic oligonucleotide
ctgttctataggttcttccccttcat

SEQ ID NO: 3 - DNA, 24 bp, Artificial Sequence
  synthetic oligonucleotide
tgcaggaggtgtgctcattggagg

SEQ ID NO: 4 - DNA, 26 bp, Artificial Sequence
  synthetic oligonucleotide
cgcggatccagtcataacaattcaag

SEQ ID NO: 5 - DNA, 25 bp, Artificial Sequence
  synthetic oligonucleotide
cgcggatccgaggttgatttttatg

SEQ ID NO: 6 - DNA, 24 bp, Artificial Sequence
  synthetic oligonucleotide
cgcaggatcgaagctcttgcctcg

SEQ ID NO: 7 - DNA, 25 bp, Artificial Sequence
  synthetic oligonucleotide
catcaatctggatccactatgtccc

SEQ ID NO: 8 - DNA, 18246 bp, Nipah virus
accaaacaagggagaatatggatacgttaaaatatataacgtatttttaaaacttaggaaccaagacaaacacttttggtcttggtattggatcctcaagaaatatatcatcatgagtgatatctttgaagaggcggctagttttaggagttatcaatctaagttagggagagatgggagggctagtgcagcaactgctactttgacaaccaagataaggatatttgtaccagctactaatagtccagagctcagatgggaactaacattgtttgcacttgatgtgattagatctccgagtgctgccgagtcaatgaaagttggagctgctttcacactcatctctatgtattcagagagacccggggctctcattagaagtctcctcaatgacccagacattgaagctgtaataatagatgttggatcaatggtcaacggaataccagtaatggagaggagaggagacaaggctcaggaggagatggaaggcttgatgagaatcctcaaaactgctcgagacagcagcaagggaaaaacaccttttgttgacagccgagcttacggcctacggataacagacatgagcaccctggtctctgcagttatcaccatcgaggcccagatctggatactgatcgctaaagcagttacagctcccgacactgccgaggaaagtgaaactagaagatgggctaaatacgtccaacaaaagagagtcaatccgttctttgctctaactcagcaatggctaacagaaatgaggaatctgctctcccagagtctatcagtaaggaagttcatggttgagatcctcatagaagtcaagaaaggaggatctgctaaaggcagagcagtagaaataatctcagacatcggaaactatgtcgaggaaactggtatggcaggattcttcgcaaccatcagattcgggttggagacaaggtatccagcacttgcactcaacgaattccagagtgacctcaacaccatcaaaagcttgatgctactctacagagaaattggcccaagagccccttatatggtgcttcttgaagaatcaattcagactaaatttgcccctggaggttacccattattgtggagctttgccatgggtgtggctactactattgacaggtctatgggggcattgaatatcaatcgtggttatcttgagcctatgtatttcagactaggccaaaaatcagcacgtcaccatgctggaggaattgatcagaacatggcaaatagactgggactaagttcagatcaagttgcagaactcgctgctgcagttcaggaaacatcagcaggaaggcaagagagtaatgttcaggctagagaggcaaaatttgctgcaggaggtgtgctcattggaggcagtgatcaagatatcgatgaaggggaagaacctatagaacagagtggcagacagtcagttaccttcaaaagggagatgagtatttcatcccttgctaacagtgtgccgagcagttctgtgagcacatccggtgggaccagattgactaattcattactaaacctcagatcaagactggctgcaaaagcagcaaaagaagccgcctcatccaatgcaacagatgatccagcaatcagcaacagaactcaaggggaatcagagaagaagaataatcaagacctcaaacctgctcaaaatgaccttgatttcgtcagagctgatgtgtgacgtctatttccaatattctacagtatccaaaaatctttctatagtacactatcataatacgacactaagggatcaaccatatcaaagttacgaatcgttttaattatattaatcaaatgatactcttttatgggcaaaccgaagaaccaatgtctacatgtaaattgagctttggtattgcaatctaatacttgctcaaaatcttgaactattagtgtaatttctatcatcatagagttatcaagattttattatataagttggtgcagatctttggacatgaattacacactacactctaatgaagacaaaatttacattacatatttaaggactatttcctatcctttcaatggtacttggttatgaaggtttcttaatttaactaagctactgtctttgcactggaatatacaatacctcttacctcatttcttactttaatatcatgttatttttttgataagtcacttaacttgaccaaggtctaccaggtaatgctcgcacaagtgaactgcaatctcaacttagattaaacataatcatgcaaaatcactattttgtactactaactcattaagaaaaacttaggatccaagagatttactctaggatctcctattaagcttagcagtcattagttgagagttcaacttgcaaaactctaaccttcactctaataacaattcatccaatggataaattggaactagtcaatgatggcctcaatattattgactttattcagaagaaccaaaaagaaatacagaagacatacggacgatcaagtattcaacaacccagcatcaaagatcaaacaaaagcctgggaagattttctgcagtgcaccagtggagaatctgaacaagttgaggggggaatgtctaaggatgatggagatgttgaaagaagaaacttggaggatctatccagtacttctcccacagatggaactattggaaagagagtgtcgaacacccgtgactgggcagaaggttcagatgacatacaactggacccagtggttacagacgttgtataccatgatcatggaggagaatgtaccggatatggatttacttcaagccctgagagagggtggagtgattacacatcaggagcaaacaatgggaatgtatgtcttgtatctgatgcaaagatgctgtcctatgctcccgaaattgcagtttctaaagaagatcgggaaactgatctagttcatcttgagaataaactatctactacaggactgaatcccacagcagtaccgttcactctgagaaacctgtctgatcctgcaaaagactctcctgtgattgctgaacactactacggactaggagttaaagagcaaaacgttggccctcagactagcagaaatgtcaatttggacagcatcaaattgtacacatcagatgacgaagaggcagatcagcttgaattcgaagatgagtttgcaggaagctcaagtgaagtgatagtcggcatttctcctgaagatgaagagccttcaagtgttggcggaaaacccaatgaatccattggacgtacaatcgaaggccaatcaatccgagacaaccttcaagccaaggacaacaaatcaacagatgtaccaggagcaggaccgaaagattcagcagtgaaggaagaaccaccccagaagaggctacctatgttagctgaagaatttgagtgctctggatcggaagacccaatcattcgggagctgctgaaggagaactcactcataaattgtcagcaagggaaagatgctcagcctccatatcattggagcatcgagaggtcaataagcccggataaaactgagatcgtcaacggtgctgtgcaaactgctgacaggcaaagaccaggaactccgatgccaaagtcccgaggtattcccattaaaaagggcacagacgcgaaatatccatctgctgggacggaaaacgtgcctgggtcgaagagtggtgcaacccggcatgttcgaggatcacccccctaccaagaaggcaagagtgtcaatgcggagaatgtccaactgaatgcttccactgcggttaaggaaactgataagtcagaagtaaaccccgtagacgacaacgactcacttgatgataaatacatcatgccttcagatgatttctcaaacactttcttcccgcacgacactgatcgcttgaattatcacgcagatcatttaggtgattatgaccttgaaaccctgtgtgaagagtcggttctaatgggagtgatcaactctataaaattaattaatctggatatgcgcttaaatcacattgaagaacaagttaaagagatcccaaagatcatcaataagcttgagtccattgacagagttctggccaagactaacaccgcactctcaaccattgaaggacacctggtttccatgatgataatgataccagggaaagggaaaggagaaagaaaggggaaaaataatcctgagcttaaaccagtgataggaagagacattctagagcagcaatctcttttttcttttgacaatgtcaagaatttcagagatggatcgttgacaaacgaaccgtatggggcagctgtacagttgagagaagatcttattcttcctgaacttaattttgaggagacaaatgcatctcaatttgttcctatggcagatgattcatccagagatgttatcaagacattgataaggactcacattaaagatagagagttgagatcagaactgattggttacctgaataaagcggaaaatgatgaggaaattcaggagatagcgaacactgtcaatgacatcattgacggtaatatttgatcactgaattgtcagcagaaatacaatgatctaacaacaatctcccacaagtagacaatggtttcaggtcaataataacaacctcaatactaatctttcacataagcattactcattccagccctcagacgataacacaatacttgatacatgtttattgaagtgtatgtagcatgattgaactattcaataactgtatttctcactcttgctcttagttagtcattgtgtctaataattattattacagtacaaggtattatgaattcaaagatacgcaataaatctgatatcagcatagagtagaaaattgttgtttttgtcatgatcattcgaagatttaacaatgatgtcaactttcatacctaaacataataacataaaatggtcgatttgtattgtagatctctcacgcattttagtgtcatgaattagtgtttcaaatcagttgcatatcaattaagaaaaacttaggagacaggtatagaacctctctttcagataactggtcaattaaggacagaaattctgtttctcaaatccgctagcctttgtcaaagaggacacaagcaatggagccggacatcaagagtatttcaagtgagtcaatggaaggagtatctgatttcagccctagttcttgggagcatggtgggtatcttgataaggttgaaccagaaattgatgaaaatggcagtatgattccaaaatacaagatctataccccaggagctaacgagaggaaatacaacaactacatgtaccttatatgttacggctttgttgaagatgttgagagaaccccagagacagggaaacgcaagaagatcaggacaattgctgcctaccctctgggtgttggtaagagtgcctctcatccccaagatcttctggaggaactctgttccctcaaagttactgtgagaagaacagctggatcaactgagaaaattgtgtttggatcatctggccctctaaatcacctcgttccgtggaagaaagtactgactagtggttcaatttttaatgcagtcaaggtttgtcggaacgttgatcagatacagcttgacaagcatcaagctctgagaatattttttctcagtatcacaaagctcaatgattctggaatctacatgattccacgaaccatgcttgagttcaggagaaacaatgccattgccttcaatcttctagtgtacttgaagattgatgctgatttatccaaaatggggatccagggaagcctcgataaagatggcttcaaggttgcctccttcatgctacacttggggaactttgtccgtcgtgcagggaagtattactctgttgattattgtaggaggaagattgataggatgaaattgcagttttcactgggttccataggcggactaagtctccacattaagatcaatggtgtaatcagcaaacggctgtttgctcaaatgggattccaaaaaaacctttgtttctctttgatggacatcaatccttggctcaacagattgacctggaacaacagttgtgagatcagccgagtagcagctgtgttgcagccttctattccaagagagttcatgatctatgatgatgtcttcattgacaatacagggagaattctaaagggctaaacagaattcttctaaaatttaatcagtcatgagtttagtaatcatacctagtcataatacatcacacaggactatttacaaaagacagttaaaaaatggaataatcatgtagtagtaattgagaacattattagaatagtataactaaaatgtagtttttttgagtatttgatttaaaattagataactattacaaaaaacttaggagccaagctcttgcctcgttcagaaggttaaacaagcattcttaccattggatcaacaaaaggattggttttatcgtctaagaaatttattgaaaggcaaagaaattcctggttttatgttgaatgaggtgtatcaaactaaggagaccttctaacagccaggtcataggaatataaataaaaataagaataaaattgattccatcggaagattcatttcaagaagtgatcaaatcaaagcggttggcagacctaccaatcatataccacaagactcgacaatggtagttatacttgacaagagatgttattgtaatcttttaatattgattttgatgatctcggagtgtagtgttgggattctacattatgagaaattgagtaaaattggacttgtcaaaggagtaacaagaaaatacaagattaaaagcaatcctctcacaaaagacattgttataaaaatgattccgaatgtgtcgaacatgtctcagtgcacagggagtgtcatggaaaattataaaacacgattaaacggtatcttaacacctataaagggagcgttagagatctacaaaaacaacactcatgaccttgtcggtgatgtgagattagccggagttataatggcaggagttgctattgggattgcaaccgcagctcaaatcactgcaggtgtagcactatatgaggcaatgaagaatgctgacaacatcaacaaactcaaaagcagcattgaatcaactaatgaagctgtcgttaaacttcaagagactgcagaaaagacagtctatgtgctgactgctctacaggattacattaatactaatttagtaccgacaattgacaagataagctgcaaacagacagaactctcactagatctggcattatcaaagtacctctctgatttgctttttgtatttggccccaaccttcaagacccagtttctaattcaatgactatacaggctatatctcaggcattcggtggaaattatgaaacactgctaagaacattgggttacgctacagaagactttgatgatcttctagaaagtgacagcataacaggtcaaatcatctatgttgatctaagtagctactatataattgtcagggtttattttcctattctgactgaaattcaacaggcctatatccaagagttgttaccagtgagcttcaacaatgataattcagaatggatcagtattgtcccaaatttcatattggtaaggaatacattaatatcaaatatagagattggattttgcctaattacaaagaggagcgtgatctgcaaccaagattatgccacacctatgaccaacaacatgagagaatgtttaacgggatcgactgagaagtgtcctcgagagctggttgtttcatcacatgttcccagatttgcactatctaacggggttctgtttgccaattgcataagtgttacatgtcagtgtcaaacaacaggcagggcaatctcacaatcaggagaacaaactctgctgatgattgacaacaccacctgtcctacagccgtactcggtaatgtgattatcagcttagggaaatatctggggtcagtaaattataattctgaaggcattgctatcggtcctccagtctttacagataaagttgatatatcaagtcagatatccagcatgaatcagtccttacaacagtctaaggactatatcaaagaggctcaacgactccttgatactgttaatccatcattaataagcatgttgtctatgatcatactgtatgtattatcgatcgcatcgttgtgtatagggttgattacatttatcagttttatcattgttgagaaaaagagaaacacctacagcagattagaggataggagagtcagacctacaagcagtggggatctctactacattgggacatagtgtattcagattgatgaaattatgttagagaaatcagaaaacttctgactttcagaaatggattgtatacaattagttagatcatcctgaataatcgaggtgagaacattgcaactataaaatcagatcatgtaaatagttgtaaaaaattaaaagcttcttttaattcttttgaacaataatttaattaatatataacatattctctcacacgagcgctaacctatacactctctactaatattttatactcataattaatgatataatgacaaataaggattcaaattggattatgatatagtttcatactacaatagcatttcgaccaagaaaatatccttacaattatacaatgtacttaaccgtgaatatgtaattgataatttccctttagaaatttaataaaaaacttaggacccaggtccataactcattggatacttaactgtatctttctaagctatcacatatcaaaggagagattgaatgcttttttggagatctagatcattactatatgtgtctcctataatcacatcataggagtgaaccataatacacatctttgggtaggggaaggaaagtattgttgacgtactgattgatctgcttgagtcaaataatcagtcataacaattcaagaaaatgccggcagaaaacaagaaagttagattcgaaaatactacttcagacaaagggaaaattcctagtaaagttattaagagctactacggaaccatggacattaagaaaataaatgaaggattattggacagcaaaatattaagtgctttcaacacagtaatagcattgcttggatctatcgtgatcatagtgatgaatataatgatcatccaaaattacacaagatcaacagacaatcaggccgtgatcaaagatgcgttgcagggtatccaacagcagatcaaagggcttgctgacaaaatcggcacagagatagggcccaaagtatcactgattgacacatccagtaccattactatcccagctaacattgggctgttaggttcaaagatcagccagtcgactgcaagtataaatgagaatgtgaatgaaaaatgcaaattcacactgcctcccttgaaaatccacgaatgtaacatttcttgtcctaacccactcccttttagagagtataggccacagacagaaggggtgagcaatctagtaggattacctaataatatttgcctgcaaaagacatctaatcagatattgaagccaaagctgatttcatacactttacccgtagtcggtcaaagtggtacctgtatcacagacccattgctggctatggacgagggctattttgcatatagccacctggaaagaatcggatcatgttcaagaggggtctccaaacaaagaataataggagttggagaggtactagacagaggtgatgaagttccttctttatttatgaccaatgtctggaccccaccaaatccaaacaccgtttaccactgtagtgctgtatacaacaatgaattctattatgtactttgtgcagtgtcaactgttggagaccctattctgaatagcacctactggtccggatctctaatgatgacccgtctagctgtgaaacccaagagtaatggtgggggttacaatcaacatcaacttgccctacgaagtatcgagaaagggaggtatgataaagttatgccgtatggaccttcaggcatcaaacagggtgacaccctgtattttcctgctgtaggatttttggtcaggacagagtttaaatacaatgattcaaattgtcccatcacgaagtgtcaatacagtaaacctgaaaattgcaggctatctatggggattagaccaaacagccattatatccttcgatctggactattaaaatacaatctatcagatggggagaaccccaaagttgtattcattgaaatatctgatcaaagattatctattggatctcctagcaaaatctatgattctttgggtcaacctgttttctaccaagcgtcattttcatgggatactatgattaaatttggagatgttctaacagtcaaccctctggttgtcaattggcgtaataacacggtaatatcaagacccgggcaatcacaatgccctagattcaatacatgtccagagatctgctgggaaggagtttataatgatgcattcctaattgacagaatcaattggataagcgcgggtgtattccttgacagcaatcagaccgcagaaaatcctgtttttactgtattcaaagataatgaaatactttatagggcacaactggcttctgaggacaccaatgcacaaaaaacaataactaattgttttctcttgaagaataagatttggtgcatatcattggttgagatatatgacacaggagacaatgtcataagacccaaactattcgcggttaagataccagagcaatgtacataaaaatcaacctcataatttaatggattgatctaatataatgataataatcgtacaaagacatgtgatgtaaacaaaattgttgtaattaaataagtcctcagctgaatacttttttaagattagcaatagcatgtttttccagttattggatagttgataatataattctgaaactgggttaataaataatcttgatcggtgatctttgagaacaatgatatcatatagttcatcaagtgataatcaattctttatatgtacactttagagtatattttgagacttagtattttcggcccgaatgttaaatttaatagttcatacataacctaaactcaagttctaagcataatgataacaattaatgcgaacttgtcttgatgtaaggaagatttgatattaactgagactccacttgatatagtagagctgaatcttgtaaataaattataatgaatagtttattcaaagattatcattcatattagtgtaaattaagaaaaacttaggacccaggtccttgattatgccaattttctcgagaaatcattcaattgaccatagactgaaagcgttgttacctagttcttcagaagagatcttattagaattaatttatatgatctaattcccttaaaaactgaataccaaaaaacaaaaatggccgatgaattatcaatatccgacatcatttaccctgaatgtcatttggatagtcctatagtctctggtaaactaatatcagctattgaatatgctcaattgagacacaatcagcccagtgatgataaaagactgtctgagaatattaggttaaaccttcacgggaaaagaaagagtctatacatattaagacaatccaaacagggtgattacattagaaacaacataaaaaacctaaaggaattcatgcatattgcgtaccctgaatgcaataacattctattctccatcacatcccaaggcatgactagcaaacttgataacatcatgaaaaagtcattcaaagcatacaatatcattagtaagaaagtaattgggatgctgcaaaatatcactagaaatctcataactcaagatagaagagatgaaataattaatatacatgagtgtaggcgattaggggatttagggaagaatatgagtcaatctaaatggtatgagtgttttttgttttggtttactatcaaaacagagatgcgagcagtgatcaagaattcgcaaaagccgaaattccgttcagattcatgcataatacacatgcgagacaaaagtactgaaataatcctaaatccgaatcttatctgcattttcaaatcagacaaaactggaaagaagtgttattatcttacacccgaaatggttctaatgtattgtgatgtcctagagggaaggatgatgatggagacaacagtcaaatcggatatcaagtaccaacctctaatctcgagatccaatgccctctgggggctaattgatcccttgttccctgtcatgggaaacagaatttacaatatagtgtctatgatagagcctttagttcttgcactactccaactcaaggatgaggctaggatcctgaggggtgcatttctgcatcactgcataaaggaaatgcatcaagaattgagtgagtgtggttttacagatcagaagattcggtctatgtttattgatgatcttttatccattctaaatatcgataatatacatctgttggcagagttcttttctttctttcgtacgtttggccatcctattcttgaggctaaagttgctgcagaaaaagtgagagaacatatgttggcagataaagttcttgaatatgcccctataatgaaagcacatgctatattctgcgggactataataaatgggtatagggatagacacggaggagcctggcctcctctttacctccccgcacatgcatctaaacatataatccgtttgaaaaattctggggaatctttgaccattgatgactgtgtcaagaattgggaatcattctgtgggattcaatttgattgtttcatggagctgaaattggacagtgatctgagtatgtatatgaaagataaagctttatctccaatcaaagacgaatgggacagtgtatacccacgtgaagtgttgagctataccccaccgaagtcaaccgagccaagaagattggttgacgtttttgtaaatgatgaaaactttgatccatacaacatgctggaatatgtcttatccggtgcttatctcgaggatgaacaattcaatgtttcttatagcttgaaggagaaagagacgaagcaagctggacgattgttcgcaaagatgacctacaaaatgcgtgcatgtcaagtcatagcagaggccctgatagcctcaggtgtcggtaaatattttaaggagaacgggatggttaaggatgagcacgaacttttgaagacactcttccaattgtctatttcctcagttcctcgagggaacagtcagggtaatgatcctcaatccatcaataatatagaaagagatttccaatactttaaaggggtcactaccaatgtgaaagacaaaaagaataactcttttaataaggttaaatctgctctcaataatccgtgccaagctgacggagtccatcataacatgtcacccaatacacgaaatcgttataagtgtagtaatacaagtaagtcttttctcgattatcataccgagtttaatcctcacaatcactataaatcagacaatacagaggcggccgtactgtccaggtatgaggacaacactgggacaaaatttgatacagtaagtgcatttcttacaactgatcttaagaaattctgtctcaattggagatacgaatcaatggctatatttgctgaacgtctggatgagatatacggtttacctggattttttaattggatgcacaaacgactagaaagatctgttatctatgttgcagaccctaattgcccccctaatattgacaaacatatggaactagaaaaaactcctgaagatgatatattcattcattatcctaaaggcggtattgaaggatatagccaaaaaacatggactatagcaactatcccctttttattcttgagtgcctatgagacaaacacgaggattgctgcaattgtccaaggagacaatgaatcaattgctatcactcaaaaagttcatcctaatcttccctacaaggtaaagaaagagatctgtgcaaagcaagctcagctttattttgaaaggttaaggatgaacttaagagccctcggccacaatcttaaagctacagaaactatcatcagtacacatctttttatttattcgaagaaaattcattatgatggtgctgtgctgtctcaggcactcaaatcaatgtcaagatgttgcttttggtcagagactctggtggatgaaactagatcagcttgtagtaacatcagcactacaatagctaaagctatagaaaatgggttgtcaagaaatgtcggctattgcatcaatattttgaaagtaattcagcagcttctcatatcaactgagtttagtattaacgagacattgacactggatgtgacatctcccatttcaaataatttagattggcttataacagctgcattaatcccggcacctattggaggattcaattaccttaatttgtctagaatttttgttagaaatataggtgatccggttacagcatctttggctgatcttaagagaatgattgatcacagtattatgactgaaagcgtattacaaaaagttatgaatcaagaacctggtgatgcgagtttcttggactgggccagtgatccatactcgggcaacttgcctgactcacaaagcatcactaaaacaattaaaaatatcacagcaaggactatactgaggaactcaccgaacccaatgctaaaaggtttatttcatgacaaatcttttgatgaagatcttgaactagctagcttcttaatggacaggagggttatattacctagagccgctcatgagatactggataattcattgacaggtgccagagaggaaattgctggtttattagatacaactaaaggcttgatcagatcagggctaagaaagagtggacttcagccaaagttagtttctagattatctcatcatgattataatcaatttttaatactgaacaaacttctatcaaacagaagacaaaatgacttgatatcatcaaatacttgctcagttgacttggcacgagcattgagatctcacatgtggagggaattagcgttaggtagagtaatatacggtcttgaggtaccagatgcacttgaggctatggtgggaaggtatataacagggagcttagagtgccaaatttgtgagcagggaaacacgatgtatgggtggttctttgtacctagggattcccaattggatcaggtagatagagagcactcatcaataagagtaccttatgtaggatcaagtacggatgaaagatcggatatcaaactagggaatgtcaaaagaccaactaaggccttgcgttctgctatcagaattgcgacagtatatacttgggcctatggggacaatgaagagtgttggtatgaagcttggtacctagcgtctcagagggtaaacatagacttagatgtattgaaagctataaccccagtttccacttcaaacaatttatcccatagattgagagataaatccacacaatttaagtttgcagggagtgtactcaacagagtttctagatatgttaacataagcaatgacaatctagatttcagaattgagggagaaaaggtagatacgaatcttatttatcaacaagcaatgctattagggttatcggtattggaaggtaaattcagattgagattagaaactgatgattacaacgggatatatcacttacacgtaaaggataattgttgtgtcaaagaagtggctgatgtaggccaagtagacgctgagttgcctatcccagaatatactgaagtggataacaatcatcttatatatgatccagaccccgtttcagaaatagattgcagccgtctttctaatcaggagtccaaatcaagagaattagactttcctttatggtcaactgaggaacttcatgatgtcctagctaagactgttgctcagaccgttcttgagattataacaaaggctgacaaggatgttttaaagcaacaccttgcaatagactctgacgataacatcaacagcttaatcacagaatttctaatagttgatcctgaactgtttgcactttatctaggacaatctatatcaataaaatgggcctttgaaattcatcataggcgtcctagaggaagacatactatggtcgacctattgtcagatcttgtatcaaatacatcaaagcacacttacaaagtgttgtcaaatgccttgtcacatcctagagtattcaagagatttgtaaactgtggcttgctattgcctacacagggtccttaccttcatcaacaagattttgaaaagttgtctcaaaaccttcttgtaacatcttatatgatttatctaatgaactggtgtgacttcaagaaatccccctttttaatcgccgaacaggatgaaactgtgataagtctacgagaggatataataacatccaaacatctctgtgttataattgacttatatgcaaatcaccataaacctccttggataatagatctaaacccacaagaaaaaatatgtgtactgcgtgactttatttctaaatctaggcatgtggacacgtcctccagatcatggaatacttctgacctggattttgtaatattctatgcatctttgacttatttgagaagaggtataataaaacaattaaggataagacaagttactgaggttatagataccacaacaatgttaagggacaatataattgtagagaatcctcctattaaaacaggagtgttagacatcagaggttgtataatatacaatttagaggaaatcctgtctatgaacacaaaatcagcatcaaaaaagatctttaatcttaatagtaggccgtcagtggagaatcataaatatagaaggataggtctcaactcatcatcttgttacaaggcattaaatctatcacctctgattcaaaggtatttgccgtcgggagctcaaaggttgtttataggagaaggttctgggagcatgatgttattatatcagtctacattggggcaatcaatttctttttacaattcaggtatagatggagattatataccaggtcaaagagaactgaaactatttccctctgaatactcaattgctgaggaagacccatctctgacggggaaattgaaaggactagtggtgcccctattcaatggaagaccagaaacaacatggatcgggaatttagactcctacgagtatatcataaataggacagcggggcgaagtataggtcttgtccattctgacatggagtctgggattgacaaaaatgtagaggagatactagtagaacattcccatctaatatctatcgcgataaatgttatgatggaggacggactattagtatccaagatagcatacacccctggattcccaatctcaagattatttaacatgtacagatcatatttcggactagtactggtgtgtttcccagtatatagtaatccagattctactgaagtatatcttctttgcttacagaagacggtcaagactattgttcccccgcaaaaagtccttgagcactctaatttgcacgatgaagtcaatgaccagggaataacatcagtgatttttaaaatcaagaattcacagtctaagcagttccacgatgatctaaagaagtactatcagattgaccaacctttttttgtaccaactaaaatcactagtgatgaacaagtacttctccaagcagggctgaaactcaatgggccagaaattcttaagagtgaaatcagttatgatatcggttcagatatcaatacattaagagacaccatcataattatgttaaatgaggctatgaattattttgatgacaacagatcaccttcacaccacctagaaccctatccagttttggagagaactagaattaaaacaataatgaattgtgtgactaaaaaagtgattgtctactcacttatcaagttcaaggacaccaaaagctcagaactttatcacatcaaaaataacatcagaagaaaagttctaatcttagatttcagatcgaagctcatgacaaagactctacctaaagggatgcaagagagaagagaaaaaaacggtttcaaagaagtttggatagtagatttatcgaatcgagaagttaaaatctggtggaagataatcggatacatatctattatctgatttaaccttccaaatccaagaccaactgataacttatgttgatctaaggttcagttattaagaaaaacttaataacgattcttctttacccttgttcggt

SEQ ID NO: 9 - protein, 2244 aa, Nipah virus
MADELSISDIIYPECHLDSPIVSGKLISAIEYAQLRHNQPSDDKRLSENIRLNLHGKRKSLYILRQSKQGDYIRNNIKNLKEFMHIAYPECNNILFSITSQGMTSKLDNIMKKSFKAYNIISKKVIGMLQNITRNLITQDRRDEIINIHECRRLGDLGKNMSQSKWYECFLFWFTIKTEMRAVIKNSQKPKFRSDSCIIHMRDKSTEIILNPNLICIFKSDKTGKKCYYLTPEMVLMYCDVLEGRMMMETTVKSDIKYQPLISRSNALWGLIDPLFPVMGNRIYNIVSMIEPLVLALLQLKDEARILRGAFLHHCIKEMHQELSECGFTDQKIRSMFIDDLLSILNIDNIHLLAEFFSFFRTFGHPILEAKVAAEKVREHMLADKVLEYAPIMKAHAIFCGTIINGYRDRHGGAWPPLYLPAHASKHIIRLKNSGESLTIDDCVKNWESFCGIQFDCFMELKLDSDLSMYMKDKALSPIKDEWDSVYPREVLSYTPPKSTEPRRLVDVFVNDENFDPYNMLEYVLSGAYLEDEQFNVSYSLKEKETKQAGRLFAKMTYKMRACQVIAEALIASGVGKYFKENGMVKDEHELLKTLFQLSISSVPRGNSQGNDPQSINNIERDFQYFKGVTTNVKDKKNNSFNKVKSALNNPCQADGVHHNMSPNTRNRYKCSNTSKSFLDYHTEFNPHNHYKSDNTEAAVLSRYEDNTGTKFDTVSAFLTTDLKKFCLNWRYESMAIFAERLDEIYGLPGFFNWMHKRLERSVIYVADPNCPPNIDKHMELEKTPEDDIFIHYPKGGIEGYSQKTWTIATIPFLFLSAYETNTRIAAIVQGDNESIAITQKVHPNLPYKVKKEICAKQAQLYFERLRMNLRALGHNLKATETIISTHLFIYSKKIHYDGAVLSQALKSMSRCCFWSETLVDETRSACSNISTTIAKAIENGLSRNVGYCINILKVIQQLLISTEFSINETLTLDVTSPISNNLDWLITAALIPAPIGGFNYLNLSRIFVRNIGDPVTASLADLKRMIDHSIMTESVLQKVMNQEPGDASFLDWASDPYSGNLPDSQSITKTIKNITARTILRNSPNPMLKGLFHDKSFDEDLELASFLMDRRVILPRAAHEILDNSLTGAREEIAGLLDTTKGLIRSGLRKSGLQPKLVSRLSHHDYNQFLILNKLLSNRRQNDLISSNTCSVDLARALRSHMWRELALGRVIYGLEVPDALEAMVGRYITGSLECQICEQGNTMYGWFFVPRDSQLDQVDREHSSIRVPYVGSSTDERSDIKLGNVKRPTKALRSAIRIATVYTWAYGDNEECWYEAWYLASQRVNIDLDVLKAITPVSTSNNLSHRLRDKSTQFKFAGSVLNRVSRYVNISNDNLDFRIEGEKVDTNLIYQQAMLLGLSVLEGKFRLRLETDDYNGIYHLHVKDNCCVKEVADVGQVDAELPIPEYTEVDNNHLIYDPDPVSEIDCSRLSNQESKSRELDFPLWSTEELHDVLAKTVAQTVLEIITKADKDVLKQHLAIDSDDNINSLITEFLIVDPELFALYLGQSISIKWAFEIHHRRPRGRHTMVDLLSDLVSNTSKHTYKVLSNALSHPRVFKRFVNCGLLLPTQGPYLHQQDFEKLSQNLLVTSYMIYLMNWCDFKKSPFLIAEQDETVISLREDIITSKHLCVIIDLYANHHKPPWIIDLNPQEKICVLRDFISKSRHVDTSSRSWNTSDLDFVIFYASLTYLRRGIIKQLRIRQVTEVIDTTTMLRDNIIVENPPIKTGVLDIRGCIIYNLEEILSMNTKSASKKIFNLNSRPSVENHKYRRIGLNSSSCYKALNLSPLIQRYLPSGAQRLFIGEGSGSMMLLYQSTLGQSISFYNSGIDGDYIPGQRELKLFPSEYSIAEEDPSLTGKLKGLVVPLFNGRPETTWIGNLDSYEYIINRTAGRSIGLVHSDMESGIDKNVEEILVEHSHLISIAINVMMEDGLLVSKIAYTPGFPISRLFNMYRSYFGLVLVCFPVYSNPDSTEVYLLCLQKTVKTIVPPQKVLEHSNLHDEVNDQGITSVIFKIKNSQSKQFHDDLKKYYQIDQPFFVPTKITSDEQVLLQAGLKLNGPEILKSEISYDIGSDINTLRDTIIIMLNEAMNYFDDNRSPSHHLEPYPVLERTRIKTIMNCVTKKVIVYSLIKFKDTKSSELYHIKNNIRRKVLILDFRSKLMTKTLPKGMQERREKNGFKEVWIVDLSNREVKIWWKIIGYISII

SEQ ID NO: 10 - protein, 602 aa, Nipah virus
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 11 - protein, 546 aa, Nipah virus
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 12 - protein, 352 aa, Nipah virus
MEPDIKSISSESMEGVSDFSPSSWEHGGYLDKVEPEIDENGSMIPKYKIYTPGANERKYNNYMYLICYGFVEDVERTPETGKRKKIRTIAAYPLGVGKSASHPQDLLEELCSLKVTVRRTAGSTEKIVFGSSGPLNHLVPWKKVLTSGSIFNAVKVCRNVDQIQLDKHQALRIFFLSITKLNDSGIYMIPRTMLEFRRNNAIAFNLLVYLKIDADLSKMGIQGSLDKDGFKVASFMLHLGNFVRRAGKYYSVDYCRRKIDRMKLQFSLGSIGGLSLHIKINGVISKRLFAQMGFQKNLCFSLMDINPWLNRLTWNNSCEISRVAAVLQPSIPREFMIYDDVFIDNTGRILKG

SEQ ID NO: 13 - protein, 166 aa, Nipah virus
MMASILLTLFRRTKKKYRRHTDDQVFNNPASKIKQKPGKIFCSAPVENLNKLRGECLRMMEMLKEETWRIYPVLLPQMELLERECRTPVTGQKVQMTYNWTQWLQTLYTMIMEENVPDMDLLQALREGGVITHQEQTMGMYVLYLMQRCCPMLPKLQFLKKIGKLI

SEQ ID NO: 14 - protein, 456 aa, Nipah virus
MDKLELVNDGLNIIDFIQKNQKEIQKTYGRSSIQQPSIKDQTKAWEDFLQCTSGESEQVEGGMSKDDGDVERRNLEDLSSTSPTDGTIGKRVSNTRDWAEGSDDIQLDPVVTDVVYHDHGGECTGYGFTSSPERGWSDYTSGANNGNVCLVSDAKMLSYAPEIAVSKEDRETDLVHLENKLSTTGLNPTAVPFTLRNLSDPAKDSPVIAEHYYGLGVKEQNVGPQTSRNVNLDSIKLYTSDDEEADQLEFEDEFAGSSSEVIVGISPEDEEPSSVGGKPNESIGRTIEGQSIRDNLQAKDNKSTDVPGAGPKDSAVKEEPPQKRLPMLAEEFECSGSEDPIIRELLKENSLINCQQGKDAQPPYHWSIERSISPDKTEIVNGAVQTADRQRPGTPMPKSRGIPIKKGHRREISICWDGKRAWVEEWCNPACSRITPLPRRQECQCGECPTECFHCG

SEQ ID NO: 15 - protein, 709 aa, Nipah virus
MDKLELVNDGLNIIDFIQKNQKEIQKTYGRSSIQQPSIKDQTKAWEDFLQCTSGESEQVEGGMSKDDGDVERRNLEDLSSTSPTDGTIGKRVSNTRDWAEGSDDIQLDPVVTDVVYHDHGGECTGYGFTSSPERGWSDYTSGANNGNVCLVSDAKMLSYAPEIAVSKEDRETDLVHLENKLSTTGLNPTAVPFTLRNLSDPAKDSPVIAEHYYGLGVKEQNVGPQTSRNVNLDSIKLYTSDDEEADQLEFEDEFAGSSSEVIVGISPEDEEPSSVGGKPNESIGRTIEGQSIRDNLQAKDNKSTDVPGAGPKDSAVKEEPPQKRLPMLAEEFECSGSEDPIIRELLKENSLINCQQGKDAQPPYHWSIERSISPDKTEIVNGAVQTADRQRPGTPMPKSRGIPIKKGTDAKYPSAGTENVPGSKSGATRHVRGSPPYQEGKSVNAENVQLNASTAVKETDKSEVNPVDDNDSLDDKYIMPSDDFSNTFFPHDTDRLNYHADHLGDYDLETLCEESVLMGVINSIKLINLDMRLNHIEEQVKEIPKIINKLESIDRVLAKTNTALSTIEGHLVSMMIMIPGKGKGERKGKNNPELKPVIGRDILEQQSLFSFDNVKNFRDGSLTNEPYGAAVQLREDLILPELNFEETNASQFVPMADDSSRDVIKTLIRTHIKDRELRSELIGYLNKAENDEEIQEIANTVNDIIDGNI

SEQ ID NO: 16 - protein, 532 aa, Nipah virus
MSDIFEEAASFRSYQSKLGRDGRASAATATLTTKIRIFVPATNSPELRWELTLFALDVIRSPSAAESMKVGAAFTLISMYSERPGALIRSLLNDPDIEAVIIDVGSMVNGIPVMERRGDKAQEEMEGLMRILKTARDSSKGKTPFVDSRAYGLRITDMSTLVSAVITIEAQIWILIAKAVTAPDTAEESETRRWAKYVQQKRVNPFFALTQQWLTEMRNLLSQSLSVRKFMVEILIEVKKGGSAKGRAVEIISDIGNYVEETGMAGFFATIRFGLETRYPALALNEFQSDLNTIKSLMLLYREIGPRAPYMVLLEESIQTKFAPGGYPLLWSFAMGVATTIDRSMGALNINRGYLEPMYFRLGQKSARHHAGGIDQNMANRLGLSSDQVAELAAAVQETSAGRQESNVQAREAKFAAGGVLIGGSDQDIDEGEEPIEQSGRQSVTFKREMSISSLANSVPSSSVSTSGGTRLTNSLLNLRSRLAAKAAKEAASSNATDDPAISNRTQGESEKKNNQDLKPAQNDLDFVRADV

SEQ ID NO: 17 - DNA, 18246 bp, Nipah virus
accaaacaagggagaatatggatacgttaaaatatataacgtatttttaaaacttaggaaccaagacaaacacttttggtcttggtattggatcctcaagaaatatatcatcatgagtgatatctttgaagaggcggctagttttaggagttatcaatctaagttagggagagatgggagggctagtgcagcaactgctactttgacaaccaagataaggatatttgtaccagctactaatagtccagagctcagatgggaactaacattgtttgcacttgatgtgattagatctccgagtgctgccgagtcaatgaaagttggagctgctttcacactcatctctatgtattcagagagacccggggctctcattagaagtctcctcaatgacccagacattgaagctgtaataatagatgttggatcaatggtcaacggaataccagtaatggagaggagaggagacaaggctcaggaggagatggaaggcttgatgagaatcctcaaaactgctcgagacagcagcaagggaaaaacaccttttgttgacagccgagcttacggcctacggataacagacatgagcaccctggtctctgcagttatcaccatcgaggcccagatctggatactgatcgctaaagcagttacagctcccgacactgccgaggaaagtgaaactagaagatgggctaaatacgtccaacaaaagagagtcaatccgttctttgctctaactcagcaatggctaacagaaatgaggaatctgctctcccagagtctatcagtaaggaagttcatggttgagatcctcatagaagtcaagaaaggaggatctgctaaaggcagagcagtagaaataatctcagacatcggaaactatgtcgaggaaactggtatggcaggattcttcgcaaccatcagattcgggttggagacaaggtatccagcacttgcactcaacgaattccagagtgacctcaacaccatcaaaagcttgatgctactctacagagaaattggcccaagagccccttatatggtgcttcttgaagaatcaattcagactaaatttgcccctggaggttacccattattgtggagctttgccatgggtgtggctactactattgacaggtctatgggggcattgaatatcaatcgtggttatcttgagcctatgtatttcagactaggccaaaaatcagcacgtcaccatgctggaggaattgatcagaacatggcaaatagactgggactaagttcagatcaagttgcagaactcgctgctgcagttcaggaaacatcagcaggaaggcaagagagtaatgttcaggctagagaggcaaaatttgctgcaggaggtgtgctcattggaggcagtgatcaagatatcgatgaaggggaagaacctatagaacagagtggcagacagtcagttaccttcaaaagggagatgagtatttcatcccttgctaacagtgtgccgagcagttctgtgagcacatccggtgggaccagattgactaattcattactaaacctcagatcaagactggctgcaaaagcagcaaaagaagccgcctcatccaatgcaacagatgatccagcaatcagcaacagaactcaaggggaatcagagaagaagaataatcaagacctcaaacctgctcaaaatgaccttgatttcgtcagagctgatgtgtgacgtctatttccaatattctacagtatccaaaaatctttctatagtacactatcataatacgacactaagggatcaaccatatcaaagttacgaatcgttttaattatattaatcaaatgatactcttttatgggcaaaccgaagaaccaatgtctacatgtaaattgagctttggtattgcaatctaatacttgctcaaaatcttgaactattagtgtaatttctatcatcatagagttatcaagattttattatataagttggtgcagatctttggacatgaattacacactacactctaatgaagacaaaatttacattacatatttaaggactatttcctatcctttcaatggtacttggttatgaaggtttcttaatttaactaagctactgtctttgcactggaatatacaatacctcttacctcatttcttactttaatatcatgttatttttttgataagtcacttaacttgaccaaggtctaccaggtaatgctcgcacaagtgaactgcaatctcaacttagattaaacataatcatgcaaaatcactattttgtactactaactcattaagaaaaacttaggatccaagagatttactctaggatctcctattaagcttagcagtcattagttgagagttcaacttgcaaaactctaaccttcactctaataacaattcatccaatggataaattggaactagtcaatgatggcctcaatattattgactttattcagaagaaccaaaaagaaatacagaagacatacggacgatcaagtattcaacaacccagcatcaaagatcaaacaaaagcctgggaagattttctgcagtgcaccagtggagaatctgaacaagttgaggggggaatgtctaaggatgatggagatgttgaaagaagaaacttggaggatctatccagtacttctcccacagatggaactattggaaagagagtgtcgaacacccgtgactgggcagaaggttcagatgacatacaactggacccagtggttacagacgttgtataccatgatcatggaggagaatgtaccggatatggatttacttcaagccctgagagagggtggagtgattacacatcaggagcaaacaatgggaatgtatgtcttgtatctgatgcaaagatgctgtcctatgctcccgaaattgcagtttctaaagaagatcgggaaactgatctagttcatcttgagaataaactatctactacaggactgaatcccacagcagtaccgttcactctgagaaacctgtctgatcctgcaaaagactctcctgtgattgctgaacactactacggactaggagttaaagagcaaaacgttggccctcagactagcagaaatgtcaatttggacagcatcaaattgtacacatcagatgacgaagaggcagatcagcttgaattcgaagatgagtttgcaggaagctcaagtgaagtgatagtcggcatttctcctgaagatgaagagccttcaagtgttggcggaaaacccaatgaatccattggacgtacaatcgaaggccaatcaatccgagacaaccttcaagccaaggacaacaaatcaacagatgtaccaggagcaggaccgaaagattcagcagtgaaggaagaaccaccccagaagaggctacctatgttagctgaagaatttgagtgctctggatcggaagacccaatcattcgggagctgctgaaggagaactcactcataaattgtcagcaagggaaagatgctcagcctccatatcattggagcatcgagaggtcaataagcccggataaaactgagatcgtcaacggtgctgtgcaaactgctgacaggcaaagaccaggaactccgatgccaaagtcccgaggtattcccattaaaaagggcacagacgcgaaatatccatctgctgggacggaaaacgtgcctgggtcgaagagtggtgcaacccggcatgttcgaggatcacccccctaccaagaaggcaagagtgtcaatgcggagaatgtccaactgaatgcttccactgcggttaaggaaactgataagtcagaagtaaaccccgtagacgacaacgactcacttgatgataaatacatcatgccttcagatgatttctcaaacactttcttcccgcacgacactgatcgcttgaattatcacgcagatcatttaggtgattatgaccttgaaaccctgtgtgaagagtcggttctaatgggagtgatcaactctataaaattaattaatctggatatgcgcttaaatcacattgaagaacaagttaaagagatcccaaagatcatcaataagcttgagtccattgacagagttctggccaagactaacaccgcactctcaaccattgaaggacacctggtttccatgatgataatgataccagggaaagggaaaggagaaagaaaggggaaaaataatcctgagcttaaaccagtgataggaagagacattctagagcagcaatctcttttttcttttgacaatgtcaagaatttcagagatggatcgttgacaaacgaaccgtatggggcagctgtacagttgagagaagatcttattcttcctgaacttaattttgaggagacaaatgcatctcaatttgttcctatggcagatgattcatccagagatgttatcaagacattgataaggactcacattaaagatagagagttgagatcagaactgattggttacctgaataaagcggaaaatgatgaggaaattcaggagatagcgaacactgtcaatgacatcattgacggtaatatttgatcactgaattgtcagcagaaatacaatgatctaacaacaatctcccacaagtagacaatggtttcaggtcaataataacaacctcaatactaatctttcacataagcattactcattccagccctcagacgataacacaatacttgatacatgtttattgaagtgtatgtagcatgattgaactattcaataactgtatttctcactcttgctcttagttagtcattgtgtctaataattattattacagtacaaggtattatgaattcaaagatacgcaataaatctgatatcagcatagagtagaaaattgttgtttttgtcatgatcattcgaagatttaacaatgatgtcaactttcatacctaaacataataacataaaatggtcgatttgtattgtagatctctcacgcattttagtgtcatgaattagtgtttcaaatcagttgcatatcaattaagaaaaacttaggagacaggtatagaacctctctttcagataactggtcaattaaggacagaaattctgtttctcaaatccgctagcctttgtcaaagaggacacaagcaatggagccggacatcaagagtatttcaagtgagtcaatggaaggagtatctgatttcagccctagttcttgggagcatggtgggtatcttgataaggttgaaccagaaattgatgaaaatggcagtatgattccaaaatacaagatctataccccaggagctaacgagaggaaatacaacaactacatgtaccttatatgttacggctttgttgaagatgttgagagaaccccagagacagggaaacgcaagaagatcaggacaattgctgcctaccctctgggtgttggtaagagtgcctctcatccccaagatcttctggaggaactctgttccctcaaagttactgtgagaagaacagctggatcaactgagaaaattgtgtttggatcatctggccctctaaatcacctcgttccgtggaagaaagtactgactagtggttcaatttttaatgcagtcaaggtttgtcggaacgttgatcagatacagcttgacaagcatcaagctctgagaatattttttctcagtatcacaaagctcaatgattctggaatctacatgattccacgaaccatgcttgagttcaggagaaacaatgccattgccttcaatcttctagtgtacttgaagattgatgctgatttatccaaaatggggatccagggaagcctcgataaagatggcttcaaggttgcctccttcatgctacacttggggaactttgtccgtcgtgcagggaagtattactctgttgattattgtaggaggaagattgataggatgaaattgcagttttcactgggttccataggcggactaagtctccacattaagatcaatggtgtaatcagcaaacggctgtttgctcaaatgggattccaaaaaaacctttgtttctctttgatggacatcaatccttggctcaacagattgacctggaacaacagttgtgagatcagccgagtagcagctgtgttgcagccttctattccaagagagttcatgatctatgatgatgtcttcattgacaatacagggagaattctaaagggctaaacagaattcttctaaaatttaatcagtcatgagtttagtaatcatacctagtcataatacatcacacaggactatttacaaaagacagttaaaaaatggaataatcatgtagtagtaattgagaacattattagaatagtataactaaaatgtagtttttttgagtatttgatttaaaattagataactattacaaaaaacttaggagccaagctcttgcctcgttcagaaggttaaacaagcattcttaccattggatcaacaaaaggattggttttatcgtctaagaaatttattgaaaggcaaagaaattcctggttttatgttgaatgaggtgtatcaaactaaggagaccttctaacagccaggtcataggaatataaataaaaataagaataaaattgattccatcggaagattcatttcaagaagtgatcaaatcaaagcggttggcagacctaccaatcatataccacaagactcgacaatggtagttatacttgacaagagatgttattgtaatcttttaatattgattttgatgatctcggagtgtagtgttgggattctacattatgagaaattgagtaaaattggacttgtcaaaggagtaacaagaaaatacaagattaaaagcaatcctctcacaaaagacattgttataaaaatgattccgaatgtgtcgaacatgtctcagtgcacagggagtgtcatggaaaattataaaacacgattaaacggtatcttaacacctataaagggagcgttagagatctacaaaaacaacactcatgaccttgtcggtgatgtgagattagccggagttataatggcaggagttgctattgggattgcaaccgcagctcaaatcactgcaggtgtagcactatatgaggcaatgaagaatgctgacaacatcaacaaactcaaaagcagcattgaatcaactaatgaagctgtcgttaaacttcaagagactgcagaaaagacagtctatgtgctgactgctctacaggattacattaatactaatttagtaccgacaattgacaagataagctgcaaacagacagaactctcactagatctggcattatcaaagtacctctctgatttgctttttgtatttggccccaaccttcaagacccagtttctaattcaatgactatacaggctatatctcaggcattcggtggaaattatgaaacactgctaagaacattgggttacgctacagaagactttgatgatcttctagaaagtgacagcataacaggtcaaatcatctatgttgatctaagtagctactatataattgtcagggtttattttcctattctgactgaaattcaacaggcctatatccaagagttgttaccagtgagcttcaacaatgataattcagaatggatcagtattgtcccaaatttcatattggtaaggaatacattaatatcaaatatagagattggattttgcctaattacaaagaggagcgtgatctgcaaccaagattatgccacacctatgaccaacaacatgagagaatgtttaacgggatcgactgagaagtgtcctcgagagctggttgtttcatcacatgttcccagatttgcactatctaacggggttctgtttgccaattgcataagtgttacatgtcagtgtcaaacaacaggcagggcaatctcacaatcaggagaacaaactctgctgatgattgacaacaccacctgtcctacagccgtactcggtaatgtgattatcagcttagggaaatatctggggtcagtaaattataattctgaaggcattgctatcggtcctccagtctttacagataaagttgatatatcaagtcagatatccagcatgaatcagtccttacaacagtctaaggactatatcaaagaggctcaacgactccttgatactgttaatccatcattaataagcatgttgtctatgatcatactgtatgtattatcgatcgcatcgttgtgtatagggttgattacatttatcagttttatcattgttgagaaaaagagaaacacctacagcagattagaggataggagagtcagacctacaagcagtggggatctctactacattgggacatagtgtattcagattgatgaaattatgttagagaaatcagaaaacttctgactttcagaaatggattgtatacaattagttagatcatcctgaataatcgaggtgagaacattgcaactataaaatcagatcatgtaaatagttgtaaaaaattaaaagcttcttttaattcttttgaacaataatttaattaatatataacatattctctcacacgagcgctaacctatacactctctactaatattttatactcataattaatgatataatgacaaataaggattcaaattggattatgatatagtttcatactacaatagcatttcgaccaagaaaatatccttacaattatacaatgtacttaaccgtgaatatgtaattgataatttccctttagaaatttaataaaaaacttaggacccaggtccataactcattggatacttaactgtatctttctaagctatcacatatcaaaggagagattgaatgcttttttggagatctagatcattactatatgtgtctcctataatcacatcataggagtgaaccataatacacatctttgggtaggggaaggaaagtattgttgacgtactgattgatctgcttgagtcaaataatcagtcataacaattcaagaaaatgccggcagaaaacaagaaagttagattcgaaaatactacttcagacaaagggaaaattcctagtaaagttattaagagctactacggaaccatggacattaagaaaataaatgaaggattattggacagcaaaatattaagtgctttcaacacagtaatagcattgcttggatctatcgtgatcatagtgatgaatataatgatcatccaaaattacacaagatcaacagacaatcaggccgtgatcaaagatgcgttgcagggtatccaacagcagatcaaagggcttgctgacaaaatcggcacagagatagggcccaaagtatcactgattgacacatccagtaccattactatcccagctaacattgggctgttaggttcaaagatcagccagtcgactgcaagtataaatgagaatgtgaatgaaaaatgcaaattcacactacctcccttgaaaatccacgaatgtaacatttcttgtcctaacccactcccttttagagagtataggccacagacagaaggggtgagcaatctagtaggattacctaataatatttgcctgcaaaagacatctaatcagatattgaagccaaagctgatttcatacactttacccgtagtcggtcaaagtggtacctgtatcacagacccattgctggctatggacgagggctattttgcatatagccacctggaaagaatcggatcatgttcaagaggggtctccaaacaaagaataataggagttggagaggtactagacagaggtgatgaagttccttctttatttatgaccaatgtctggaccccaccaaatccaaacaccgtttaccactgtagtgctgtatacaacaatgaattctattatgtactttgtgcagtgtcaactgttggagaccctattctgaatagcacctactggtccggatctctaatgatgacccgtctagctgtgaaacccaagagtaatggtgggggttacaatcaacatcaacttgccctacgaagtatcgagaaagggaggtatgataaagttatgccgtatggaccttcaggcatcaaacagggtgacaccctgtattttcctgctgtaggatttttggtcaggacagagtttaaatacaatgattcaaattgtcccatcacgaagtgtcaatacagtaaacctgaaaattgcaggctatctatggggattagaccaaacagccattatatccttcgatctggactattaaaatacaatctatcagatggggagaaccccaaagttgtattcattgaaatatctgatcaaagattatctattggatctcctagcaaaatctatgattctttgggtcaacctgttttctaccaagcgtcattttcatgggatactatgattaaatttggagatgttctaacagtcaaccctctggttgtcaattggcgtaataacacggtaatatcaagacccgggcaatcacaatgccctagattcaatacatgtccagagatctgctgggaaggagtttataatgatgcattcctaattgacagaatcaattggataagcgcgggtgtattccttgacagcaatcagaccgcagaaaatcctgtttttactgtattcaaagataatgaaatactttatagggcacaactggcttctgaggacaccaatgcacaaaaaacaataactaattgttttctcttgaagaataagatttggtgcatatcattggttgagatatatgacacaggagacaatgtcataagacccaaactattcgcggttaagataccagagcaatgtacataaaaatcaacctcataatttaatggattgatctaatataatgataataatcgtacaaagacatgtgatgtaaacaaaattgttgtaattaaataagtcctcagctgaatacttttttaagattagcaatagcatgtttttccagttattggatagttgataatataattctgaaactgggttaataaataatcttgatcggtgatctttgagaacaatgatatcatatagttcatcaagtgataatcaattctttatatgtacactttagagtatattttgagacttagtattttcggcccgaatgttaaatttaatagttcatacataacctaaactcaagttctaagcataatgataacaattaatgcgaacttgtcttgatgtaaggaagatttgatattaactgagactccacttgatatagtagagctgaatcttgtaaataaattataatgaatagtttattcaaagattatcattcatattagtgtaaattaagaaaaacttaggacccaggtccttgattatgccaattttctcgagaaatcattcaattgaccatagactgaaagcgttgttacctagttcttcagaagagatcttattagaattaatttatatgatctaattcccttaaaaactgaataccaaaaaacaaaaatggccgatgaattatcaatatccgacatcatttaccctgaatgtcatttggatagtcctatagtctctggtaaactaatatcagctattgaatatgctcaattgagacacaatcagcccagtgatgataaaagactgtctgagaatattaggttaaaccttcacgggaaaagaaagagtctatacatattaagacaatccaaacagggtgattacattagaaacaacataaaaaacctaaaggaattcatgcatattgcgtaccctgaatgcaataacattctattctccatcacatcccaaggcatgactagcaaacttgataacatcatgaaaaagtcattcaaagcatacaatatcattagtaagaaagtaattgggatgctgcaaaatatcactagaaatctcataactcaagatagaagagatgaaataattaatatacatgagtgtaggcgattaggggatttagggaagaatatgagtcaatctaaatggtatgagtgttttttgttttggtttactatcaaaacagagatgcgagcagtgatcaagaattcgcaaaagccgaaattccgttcagattcatgcataatacacatgcgagacaaaagtactgaaataatcctaaatccgaatcttatctgcattttcaaatcagacaaaactggaaagaagtgttattatcttacacccgaaatggttctaatgtattgtgatgtcctagagggaaggatgatgatggagacaacagtcaaatcggatatcaagtaccaacctctaatctcgagatccaatgccctctgggggctaattgatcccttgttccctgtcatgggaaacagaatttacaatatagtgtctatgatagagcctttagttcttgcactactccaactcaaggatgaggctaggatcctgaggggtgcatttctgcatcactgcataaaggaaatgcatcaagaattgagtgagtgtggttttacagatcagaagattcggtctatgtttattgatgatcttttatccattctaaatatcgataatatacatctgttggcagagttcttttctttctttcgtacgtttggccatcctattcttgaggctaaagttgctgcagaaaaagtgagagaacatatgttggcagataaagttcttgaatatgcccctataatgaaagcacatgctatattctgcgggactataataaatgggtatagggatagacacggaggagcctggcctcctctttacctccccgcacatgcatctaaacatataatccgtttgaaaaattctggggaatctttgaccattgatgactgtgtcaagaattgggaatcattctgtgggattcaatttgattgtttcatggagctgaaattggacagtgatctgagtatgtatatgaaagataaagctttatctccaatcaaagacgaatgggacagtgtatacccacgtgaagtgttgagctataccccaccgaagtcaaccgagccaagaagattggttgacgtttttgtaaatgatgaaaactttgatccatacaacatgctggaatatgtcttatccggtgcttatctcgaggatgaacaattcaatgtttcttatagcttgaaggagaaagagacgaagcaagctggacgattgttcgcaaagatgacctacaaaatgcgtgcatgtcaagtcatagcagaggccctgatagcctcaggtgtcggtaaatattttaaggagaacgggatggttaaggatgagcacgaacttttgaagacactcttccaattgtctatttcctcagttcctcgagggaacagtcagggtaatgatcctcaatccatcaataatatagaaagagatttccaatactttaaaggggtcactaccaatgtgaaagacaaaaagaataactcttttaataaggttaaatctgctctcaataatccgtgccaagctgacggagtccatcataacatgtcacccaatacacgaaatcgttataagtgtagtaatacaagtaagtcttttctcgattatcataccgagtttaatcctcacaatcactataaatcagacaatacagaggcggccgtactgtccaggtatgaggacaacactgggacaaaatttgatacagtaagtgcatttcttacaactgatcttaagaaattctgtctcaattggagatacgaatcaatggctatatttgctgaacgtctggatgagatatacggtttacctggattttttaattggatgcacaaacgactagaaagatctgttatctatgttgcagaccctaattgcccccctaatattgacaaacatatggaactagaaaaaactcctgaagatgatatattcattcattatcctaaaggcggtattgaaggatatagccaaaaaacatggactatagcaactatcccctttttattcttgagtgcctatgagacaaacacgaggattgctgcaattgtccaaggagacaatgaatcaattgctatcactcaaaaagttcatcctaatcttccctacaaggtaaagaaagagatctgtgcaaagcaagctcagctttattttgaaaggttaaggatgaacttaagagccctcggccacaatcttaaagctacagaaactatcatcagtacacatctttttatttattcgaagaaaattcattatgatggtgctgtgctgtctcaggcactcaaatcaatgtcaagatgttgcttttggtcagagactctggtggatgaaactagatcagcttgtagtaacatcagcactacaatagctaaagctatagaaaatgggttgtcaagaaatgtcggctattgcatcaatattttgaaagtaattcagcagcttctcatatcaactgagtttagtattaacgagacattgacactggatgtgacatctcccatttcaaataatttagattggcttataacagctgcattaatcccggcacctattggaggattcaattaccttaatttgtctagaatttttgttagaaatataggtgatccggttacagcatctttggctgatcttaagagaatgattgatcacagtattatgactgaaagcgtattacaaaaagttatgaatcaagaacctggtgatgcgagtttcttggactgggccagtgatccatactcgggcaacttgcctgactcacaaagcatcactaaaacaattaaaaatatcacagcaaggactatactgaggaactcaccgaacccaatgctaaaaggtttatttcatgacaaatcttttgatgaagatcttgaactagctagcttcttaatggacaggagggttatattacctagagccgctcatgagatactggataattcattgacaggtgccagagaggaaattgctggtttattagatacaactaaaggcttgatcagatcagggctaagaaagagtggacttcagccaaagttagtttctagattatctcatcatgattataatcaatttttaatactgaacaaacttctatcaaacagaagacaaaatgacttgatatcatcaaatacttgctcagttgacttggcacgagcattgagatctcacatgtggagggaattagcgttaggtagagtaatatacggtcttgaggtaccagatgcacttgaggctatggtgggaaggtatataacagggagcttagagtgccaaatttgtgagcagggaaacacgatgtatgggtggttctttgtacctagggattcccaattggatcaggtagatagagagcactcatcaataagagtaccttatgtaggatcaagtacggatgaaagatcggatatcaaactagggaatgtcaaaagaccaactaaggccttgcgttctgctatcagaattgcgacagtatatacttgggcctatggggacaatgaagagtgttggtatgaagcttggtacctagcgtctcagagggtaaacatagacttagatgtattgaaagctataaccccagtttccacttcaaacaatttatcccatagattgagagataaatccacacaatttaagtttgcagggagtgtactcaacagagtttctagatatgttaacataagcaatgacaatctagatttcagaattgagggagaaaaggtagatacgaatcttatttatcaacaagcaatgctattagggttatcggtattggaaggtaaattcagattgagattagaaactgatgattacaacgggatatatcacttacacgtaaaggataattgttgtgtcaaagaagtggctgatgtaggccaagtagacgctgagttgcctatcccagaatatactgaagtggataacaatcatcttatatatgatccagaccccgtttcagaaatagattgcagccgtctttctaatcaggagtccaaatcaagagaattagactttcctttatggtcaactgaggaacttcatgatgtcctagctaagactgttgctcagaccgttcttgagattataacaaaggctgacaaggatgttttaaagcaacaccttgcaatagactctgacgataacatcaacagcttaatcacagaatttctaatagttgatcctgaactgtttgcactttatctaggacaatctatatcaataaaatgggcctttgaaattcatcataggcgtcctagaggaagacatactatggtcgacttattgtcagatcttgtatcaaatacatcaaagcacacttacaaagtgttgtcaaatgccttgtcacatcctagagtattcaagagatttgtaaactgtggcttgctattgcctacacagggtccttaccttcatcaacaagattttgaaaagttgtctcaaaaccttcttgtaacatcttatatgatttatctaatgaactggtgtgacttcaagaaattcccctttttaatcgccgaacaggatgaaactgtgataagtctacgagaggatataataacatccaaacatctctgtgttataattgacttatatgcaaatcaccataaacctccttggataatagatctaaacccacaagaaaaaatatgtgtactgcgtgactttatttctaaatctaggcatgtggacacgtcctccagatcatggaatacttctgacctggattttgtaatattctatgcatctttgacttatttgagaagaggtataataaaacaattaaggataagacaagttactgaggttatagataccacaacaatgttaagggacaatataattgtagagaatcctcctattaaaacaggagtgttagacatcagaggttgtataatatacaatttagaggaaatcctgtctatgaacacaaaatcagcatcaaaaaagatctttaatcttaatagtaggccgtcagtggagaatcataaatatagaaggataggtctcaactcatcatcttgttacaaggcattaaatctatcacctctgattcaaaggtatttgccgtcgggagctcaaaggttgtttataggagaaggttctgggagcatgatgttattatatcagtctacattggggcaatcaatttctttttacaattcaggtatagatggagattatataccaggtcaaagagaactgaaactatttccctctgaatactcaattgctgaggaagacccatctctgacggggaaattgaaaggactagtggtgcccctattcaatggaagaccagaaacaacatggatcgggaatttagactcctacgagtatatcataaataggacagcggggcgaagtataggtcttgtccattctgacatggagtctgggattgacaaaaatgtagaggagatactagtagaacattcccatctaatatctatcgcgataaatgttatgatggaggacggactattagtatccaagatagcatacacccctggattcccaatctcaagattatttaacatgtacagatcatatttcggactagtactggtgtgtttcccagtatatagtaatccagattctactgaagtatatcttctttgcttacagaagacggtcaagactattgttcccccgcaaaaagtccttgagcactctaatttgcacgatgaagtcaatgaccagggaataacatcagtgatttttaaaatcaagaattcacagtctaagcagttccacgatgatctaaagaagtactatcagattgaccaacctttttttgtaccaactaaaatcactagtgatgaacaagtacttctccaagcagggctgaaactcaatgggccagaaattcttaagagtgaaatcagttatgatatcggttcagatatcaatacattaagagacaccatcataattatgttaaatgaggctatgaattattttgatgacaacagatcaccttcacaccacctagaaccctatccagttttggagagaactagaattaaaacaataatgaattgtgtgactaaaaaagtgattgtctactcacttatcaagttcaaggacaccaaaagctcagaactttatcacatcaaaaataacatcagaagaaaagttctaatcttagatttcagatcgaagctcatgacaaagactctacctaaagggatgcaagagagaagagaaaaaaacggtttcaaagaagtttggatagtagatttatcgaatcgagaagttaaaatctggtggaagataatcggatacatatctattatctgatttaaccttccaaatccaagaccaactgataacttatgttgatctaaggttcagttattaagaaaaacttaataacgattcttctttacccttgttcggt

SEQ ID NO: 18 - protein, 2244 aa, Nipah virus
MADELSISDIIYPECHLDSPIVSGKLISAIEYAQLRHNQPSDDKRLSENIRLNLHGKRKSLYILRQSKQGDYIRNNIKNLKEFMHIAYPECNNILFSITSQGMTSKLDNIMKKSFKAYNIISKKVIGMLQNITRNLITQDRRDEIINIHECRRLGDLGKNMSQSKWYECFLFWFTIKTEMRAVIKNSQKPKFRSDSCIIHMRDKSTEIILNPNLICIFKSDKTGKKCYYLTPEMVLMYCDVLEGRMMMETTVKSDIKYQPLISRSNALWGLIDPLFPVMGNRIYNIVSMIEPLVLALLQLKDEARILRGAFLHHCIKEMHQELSECGFTDQKIRSMFIDDLLSILNIDNIHLLAEFFSFFRTFGHPILEAKVAAEKVREHMLADKVLEYAPIMKAHAIFCGTIINGYRDRHGGAWPPLYLPAHASKHIIRLKNSGESLTIDDCVKNWESFCGIQFDCFMELKLDSDLSMYMKDKALSPIKDEWDSVYPREVLSYTPPKSTEPRRLVDVFVNDENFDPYNMLEYVLSGAYLEDEQFNVSYSLKEKETKQAGRLFAKMTYKMRACQVIAEALIASGVGKYFKENGMVKDEHELLKTLFQLSISSVPRGNSQGNDPQSINNIERDFQYFKGVTTNVKDKKNNSFNKVKSALNNPCQADGVHHNMSPNTRNRYKCSNTSKSFLDYHTEFNPHNHYKSDNTEAAVLSRYEDNTGTKFDTVSAFLTTDLKKFCLNWRYESMAIFAERLDEIYGLPGFFNWMHKRLERSVIYVADPNCPPNIDKHMELEKTPEDDIFIHYPKGGIEGYSQKTWTIATIPFLFLSAYETNTRIAAIVQGDNESIAITQKVHPNLPYKVKKEICAKQAQLYFERLRMNLRALGHNLKATETIISTHLFIYSKKIHYDGAVLSQALKSMSRCCFWSETLVDETRSACSNISTTIAKAIENGLSRNVGYCINILKVIQQLLISTEFSINETLTLDVTSPISNNLDWLITAALIPAPIGGFNYLNLSRIFVRNIGDPVTASLADLKRMIDHSIMTESVLQKVMNQEPGDASFLDWASDPYSGNLPDSQSITKTIKNITARTILRNSPNPMLKGLFHDKSFDEDLELASFLMDRRVILPRAAHEILDNSLTGAREEIAGLLDTTKGLIRSGLRKSGLQPKLVSRLSHHDYNQFLILNKLLSNRRQNDLISSNTCSVDLARALRSHMWRELALGRVIYGLEVPDALEAMVGRYITGSLECQICEQGNTMYGWFFVPRDSQLDQVDREHSSIRVPYVGSSTDERSDIKLGNVKRPTKALRSAIRIATVYTWAYGDNEECWYEAWYLASQRVNIDLDVLKAITPVSTSNNLSHRLRDKSTQFKFAGSVLNRVSRYVNISNDNLDFRIEGEKVDTNLIYQQAMLLGLSVLEGKFRLRLETDDYNGIYHLHVKDNCCVKEVADVGQVDAELPIPEYTEVDNNHLIYDPDPVSEIDCSRLSNQESKSRELDFPLWSTEELHDVLAKTVAQTVLEIITKADKDVLKQHLAIDSDDNINSLITEFLIVDPELFALYLGQSISIKWAFEIHHRRPRGRHTMVDLLSDLVSNTSKHTYKVLSNALSHPRVFKRFVNCGLLLPTQGPYLHQQDFEKLSQNLLVTSYMIYLMNWCDFKKFPFLIAEQDETVISLREDIITSKHLCVIIDLYANHHKPPWIIDLNPQEKICVLRDFISKSRHVDTSSRSWNTSDLDFVIFYASLTYLRRGIIKQLRIRQVTEVIDTTTMLRDNIIVENPPIKTGVLDIRGCIIYNLEEILSMNTKSASKKIFNLNSRPSVENHKYRRIGLNSSSCYKALNLSPLIQRYLPSGAQRLFIGEGSGSMMLLYQSTLGQSISFYNSGIDGDYIPGQRELKLFPSEYSIAEEDPSLTGKLKGLVVPLFNGRPETTWIGNLDSYEYIINRTAGRSIGLVHSDMESGIDKNVEEILVEHSHLISIAINVMMEDGLLVSKIAYTPGFPISRLFNMYRSYFGLVLVCFPVYSNPDSTEVYLLCLQKTVKTIVPPQKVLEHSNLHDEVNDQGITSVIFKIKNSQSKQFHDDLKKYYQIDQPFFVPTKITSDEQVLLQAGLKLNGPEILKSEISYDIGSDINTLRDTIIIMLNEAMNYFDDNRSPSHHLEPYPVLERTRIKTIMNCVTKKVIVYSLIKFKDTKSSELYHIKNNIRRKVLILDFRSKLMTKTLPKGMQERREKNGFKEVWIVDLSNREVKIWWKIIGYISII

SEQ ID NO: 19 - protein, 602 aa, Nipah virus; identical to SEQ ID NO: 10

SEQ ID NO: 20 - protein, 546 aa, Nipah virus; identical to SEQ ID NO: 11

SEQ ID NO: 21 - protein, 352 aa, Nipah virus; identical to SEQ ID NO: 12

SEQ ID NO: 22 - protein, 709 aa, Nipah virus; identical to SEQ ID NO: 15

SEQ ID NO: 23 - protein, 532 aa, Nipah virus; identical to SEQ ID NO: 16

SEQ ID NO: 24 - protein, 709 aa, Nipah virus; identical to SEQ ID NO: 15

SEQ ID NO: 25 - protein, 398 aa, Nipah virus
MDKLELVNDGLNIIDFIQKNQKEIQKTYGRSSIQQPSIKDQTKAWEDFLQCTSGESEQVEGGMSKDDGDVERRNLEDLSSTSPTDGTIGKRVSNTRDWAEGSDDIQLDPVVTDVVYHDHGGECTGYGFTSSPERGWSDYTSGANNGNVCLVSDAKMLSYAPEIAVSKEDRETDLVHLENKLSTTGLNPTAVPFTLRNLSDPAKDSPVIAEHYYGLGVKEQNVGPQTSRNVNLDSIKLYTSDDEEADQLEFEDEFAGSSSEVIVGISPEDEEPSSVGGKPNESIGRTIEGQSIRDNLQAKDNKSTDVPGAGPKDSAVKEEPPQKRLPMLAEEFECSGSEDPIIRELLKENSLINCQQGKDAQPPYHWSIERSISPDKTEIVNGAVQTADRQRPGTPMPK

SEQ ID NO: 26 - protein, 166 aa, Nipah virus; identical to SEQ ID NO: 13

SEQ ID NO: 27 - protein, 352 aa, Nipah virus; identical to SEQ ID NO: 12

SEQ ID NO: 28 - protein, 546 aa, Nipah virus; identical to SEQ ID NO: 11

SEQ ID NO: 29 - protein, 602 aa, Nipah virus; identical to SEQ ID NO: 10

SEQ ID NO: 30 - protein, 2244 aa, Nipah virus; identical to SEQ ID NO: 9

SEQ ID NO: 31 - protein, 532 aa, Nipah virus; identical to SEQ ID NO: 16

SEQ ID NO: 32 - protein, 532 aa, Nipah virus; identical to SEQ ID NO: 16

SEQ ID NO: 33 - DNA, 18234 bp, Hendra virus
accgaacaaggggaaatatggatacgtgttaaaaaactgcgtatgtttaaaacttaggaaccaagacagtgacaattggtcttggtattggacaattgttcaaggttccaaaatgagtgatatatttgacgaggcggctagtttcagaagctatcaatcgaaactcggtcgagatgggcgggcaagtgcggcaacagctactttgactactaagataagaatttttgtaccagcaactaatagcccagaactgagatgggagttgactttgttcgctctcgatgtaatcagatcaccaagtgcagcagaatcaatgaagattggtgctgctttcactctgatatcaatgtactcagaaagacctggcgctttaatcaggagcctccttaacgacccagacatagaagctgtcatcatagatgtgggttccatgctcaacgggatccctgtgatggaaaggagaggggacaaagcacaagaggagatggaaggtttgatgaggattctgaagactgcacgtgaaagcagcaagggaaagaccccgtttgtagatagcagagcctatggactgaggatcaccgatatgagcactcttgtgtcagccgtcattaccatcgaagcccaaatttggatcctgattgcaaaggcagtgactgctccagatacagccgaggaaagtgagaccagaagatgggcaaagtatgttcaacaaaagagggtcaatccattctttgccttaacccagcaatggctgacagagatgaggaatctcctctcacaaagtctctcagtcagaaaattcatggtggaaattctgatggaggtaaagaaaggcggatcagctaaaggaagggccgttgagataatatctgacataggaaattatgtcgaggaaacaggaatggccggcttctttgcgactatcagattcggtcttgaaacaagataccctgcacttgccctcaatgagttccagagtgatctcaataccatcaaagggctgatgctgctctacagagaaatagggcctcgagcgccatacatggtgctccttgaggaatctattcagacaaagtttgcaccaggtggttatccactcctgtggagctttgctatgggtgttgcaaccactatcgaccgatccatgggtgctctcaacatcaatcgtggttatcttgaacctatgtatttcagacttggacagaaatcagcaagacatcatgctggcgggattgatcaaaacatggctaataaacttggattaaattctgatcaagttgcagaactggctgctgcagttcaagaaacatcagttgggagacaggacaacaacatgcaggcaagagaagccaaatttgcagctgggggagttcttgtggggggtggcgaacaagatattgatgaggaagaagagcctatcgaacatagtggtaggcagtcagtgactttcaagagagaaatgagcatgtcatctctcgcagacagtgtcccaagcagttcagtgagtacatccggaggaacgagattgacaaattctctattgaatctcagatccagattagctgcaaaggctatcaaagagagtactgcacagagctcatcggaaagaaacccacctaataaccgccctcaggcagactcaggaaggaaagatgaccaggaacccaaacctgcccaaaatgatttggactttgttagagcagacgtgtgacgcttaatcagaaactcatataagctccaaatcattctctatgacacactcttataaataaatttaagagagtggattgagattgagttatggacctgtgtcataatatacctttgtcttttaataaattacttataccacccacatatgatgaatatgagttcgcagtagtttgtcttataatttaaatcactctaaagccatgcttgaacctcaactttggatagtcacactgcaatgaatacatttagatatcctcagagtatttaactcactaaatctcagtagtagtaagtctcactggtgatgggatacaagtgcaagttccttttcaaatggtgctcaaatctaaggatcctcttgctgaataggctatctactatgcactggaaaatataatgaactttacctcatacataactaaatgatcaaagtttgtactgagtacatctattaaagtttataacaggctaggggcaacacctagaaataatgtaagtctgttattttagataacagtatatgagtggcatgggtctagacaacttgatcattatattaagaaaaacttaggatccaagaccataaatctaggatcctttacaatctcagaccctggtgcaaggttatatatagagcccaattctcctaattaaacagtgtctcaggttgacaaatggacaagttggatctagtcaatgatggcctcgatattattgactttattcagaagaaccaaaaagaaatacaaaagacatacggacgatcaagcatccaacaaccaagtaccaaagacaggacaagagcatgggaggacttcttgcagagcaccagtggagaacatgaacaggctgaggggggaatgcctaagaatgatggaggtactgaaggaagaaatgtggaggatctatccagtgttacttcctcagatggaactattggacaaagagtgtcaaacacccgagcttgggcagaagacccagatgacatacaactggacccaatggttacagacgttgtataccatgatcatggaggagaatgtaccggacatggaccttcttcaagccctgagagagggtggagttatcacatgtcaggaacacacgatgggaatgtacgtgctgtacctgatacaaaggtgttgcccaatgctcccaaaactacagttcctgaagaagttagggaaattgatttaattgggttggaggacaaatttgcatcagcgggattaaatccagctgcagttccctttgtccccaagaatcaatcaaccccaactgaggagcctccagtcatcccggagtattactatggatctgggaggagaggagatctctcaaaatctcctcctagaggtaatgtcaatctggacagcatcaagatttacacttcggacgacgaggatgaaaatcagctggagtatgaggatgagtttgccaaaagctcaagtgaggttgtcattgacactactcctgaggacaatgattctatcaaccaggaggaagtagttggggacccgtctgatcagggtttagagcatcctttccctttggggaaattcccggagaaagaagaaactcctgatgtacgcagaaaggattccctaatgcaggacagttgtaagagaggaggagtaccgaaaagactgcccatgttatctgaagagttcgagtgctccggatcagatgatccaataatacaagaattagaacgagaagggtctcatccaggaggttcattacgtttgagggaaccacctcagtcgtcagggaattccagaaatcaacctgaccgtcagctgaagacaggtgatgcagcatcacccggaggcgtccagagaccaggcacaccgatgcctaaatcaaggatcatgcccattaaaaagggcacagacgcgaagtctcaatatgttgggacggaagacgtgcctgggtcgaagagtggtgcaacccggtatgttcgcggattaccccccaaccaagaaagcaagagtgttactgcggagaatgtccaactgagtgctcccagtgctgtcacgaggaatgaaggacacgaccaggaagttaccagcaacgaagatagtctggatgacaaatatattatgccatcagatgattttgctaacactttcttaccccatgacactgataggttaaattatcatgcagaccatctcaatgattacgatttagagaccttgtgtgaggagtcagtattgatgggcatcgttaacgcaatcaaactcatcaacattgatatgaggttgaatcacattgaggaacagatgaaagagatacccaagatcatcaacaaaatagactccattgatcgagtgttggctaagaccaatactgcattgtcgacaatagaaggacacctagtctcgatgatgatcatgataccagggaaaggcaagggtgaaaggaaagggaaaacaaatccggagctaaaacctgtaattgggagaaatatcctagaacagcaagagttattttcattcgacaatctcaagaatttcagagatgggtcattgactgatgagccctatggaggggtggcccgaataagagatgatttgatcctgcctgaactcaacttcagtgagacaaatgcatcacaatttgttcctttggcagatgatgcatctaaggatgtcgtgagaactatgattaggactcacatcaaggacagagagttgaggtctgagttgatggattatctaaatcgagcagaaacagatgaagaggttcaggaggtggccaatacagtcaatgatattattgatgggaacatctaaacatgtcagtaatttgatagagtagcagtcagaccaagatcatatattagaggtacacatcataatggccagtgtcttaatctcaaccattcataaaccgtcagtgtttcccttataaactccattatgtagtttattaatatctagagctattctttattaagtaataaatctgtattagattattatataatctctataatcaacacgcctattacttcatgcacttactatcaccatttaataatagttcagatcacccctcaaaatctgggttgtgccaagtcattttcctcaccatataatccacgcagtatgcattcaaatactaaaccacattcagacagagttagacattcaagcaatcaataactatattaactataagaatgatgaatcagccctgcgccaacaaggcaatgtcaatcacttgaatgcaggggtagaccccagaatccattaagaaaaacttaggagacaggtataatattctcatacctgtccattcaccttggacttaaagagagacaatcattcagccccgtccaagacacacctgagtggccagaaccatggattttagtgtgagtgataaccttgatgatccaatagaaggtgtttcagattttagtcccacctcatgggagaatggagggtatctagataaagtggagccagaaattgataagcatggcagtatgatacctaagtacaagatctataccccaggtgcaaatgagaggaaattcaacaactacatgtacatgatttgctatggcttcgtcgaagacgttgagagatcaccagaatctgggaagcgcaagaaaatcaggaccattgccgcgtaccctcttggtgttggcaagagcacttctcaccctcaggatcttttagaagagctatgttctttgaaagtcactgtcagaagaacagctggggctacagagaaaattgtgttcgggtcctctggcccgcttcatcatctcttaccatggaagaagattctgactggcggatcaatattcaatgctgtcaaggtttgccgcaatgtggatcagattcaactggagaatcaacaatcattgaggatttttttcttgagtattactaaattgaatgactccggcatctacatgatcccaaggacaatgctagaattcagaaggaacaacgctattgctttcaatcttctagtatacctcaaaatcgatgcagatcttgcaaaagctggaatccaaggaagcttcgacaaagacggaaccaaagtggcatctttcatgctccatctcgggaattttgtccgacgagctgggaagtactattctgtcgaatactgcaagagaaagattgacagaatgaagctccagttctctcttggttcaattgggggtctaagcttacacatcaaaattaatggagtgatcagtaagagattatttgcccagatgggtttccagaagaacctttgcttttctctaatggacatcaatccttggctcaacagactgacttggaacaacagttgtgagatcagcagggtggcagctgtactgcagccctcggtgccacgtgaattcatgatctatgacgatgtgttcattgacaacacagggaagatcctaaaggggtgaaataaaacattcacttggttattgtagattgataaattaaccagataaccactttaatcttcattctaaagaatgccacagtgtcagtaagttacacaggtacaatacataatctaaattcatgtcttcccttcaaaggattgatttggacctttaaaatagtagaggtaagaattgagtatatccaatattaagaaaaacttaggagccaagttttggtctctttagattaatcaagctgtttgtcaatctgtattcgtaaggttgttaaaataatctgtgtctatcctttagatatccacttgggttgtagatatagtcaatcaactggtctgacaagtcataccccaaacaatccaaatctggaatcacctgttcgctcagtagtaatatcttggcaaacactacactaacctcgtctgtactgtctgaaagtggtcaggattgattgacagttattatttatctttaagcaatggctacacaagaggtcaggctaaagtgtttgctctgtgggatcatagttctggttttgtcattagaagggctagggatactacattatgagaaacttagtaagatagggctggttaaaggtattacaagaaagtacaagattaagagtaaccctttgaccaaggatattgtgatcaaaatgatccctaatgtctcgaatgtctcaaagtgcaccgggactgttatggagaattacaaaagcagactcacagggattctctcaccaatcaaaggcgccatcgaactgtacaataataacacgcatgacctagttggtgatgtcaagcttgcaggtgtggtgatggcagggattgcaatcgggatagctactgctgcacaaatcacagcaggtgttgccttatatgaggcaatgaagaacgcagacaatatcaataaactcaagagcagcatagagtctacaaatgaggctgttgtcaaattacaggaaacagctgagaaaacagtctacgtccttactgctcttcaagattacatcaacactaaccttgttcctacaatagatcaaattagctgcaagcaaacagagctcgcattagacttggcgttgtctaagtatctgtctgatctgctctttgttttcggacctaacttacaggatccagtctctaattccatgactatccaagcaatatctcaagcatttgggggcaattacgaaaccttactgagaacgcttggttacgcgaccgaggacttcgacgaccttttagaaagtgatagcatagcaggccagatagtctatgtagatctcagtagctattacataatagtaagggtgtattttcccatactaacagagatccaacaggcttatgtgcaggagttgcttccagtgagttttaataacgataattcagaatggatcagcattgtcccgaatttcgtgctgattaggaacacgctgatttcaaatatagaagtcaagtactgcttaatcaccaagaaaagtgtgatttgtaatcaggactatgctacacccatgacggctagcgtgagagaatgcttgacaggatccacagataagtgcccaagggagttagtagtctcatcccatgttccaagatttgccctctcaggaggagtcttgtttgcaaattgtataagtgtgacatgtcagtgtcagactactgggagggcaatatctcaatcaggggaacagacactactgatgattgacaatactacctgcacaacagttgttctaggaaacataatcataagccttggaaaatatttgggatcaataaattacaattctgagagcattgctgttgggccaccagtctatacagacaaagttgatatctcaagtcagatatctagtatgaatcaatcactacaacaatctaaggattacattaaagaagctcaaaagatcttggacactgtgaatccgtcgttgataagtatgctatcaatgatcatcctttatgttttgtccattgcagcactgtgcattggtctgatcactttcataagctttgtaatagttgagaaaaagagagggaattacagcaggctagatgataggcaagtgcgaccggtcagtaatggtgatctgtattatattggaacataaaatacactacaatcaatccaattgttttatctttactaatgatataaacgatacttaatcatcagctacctaagtgctcatatagctctcatgtatattcttttattattgttaacaacaggtctactaaatagtttaatccaataaattgaggataatcaattcttgcattgcataaactcgttaggttatatataacaaaaattatttttttctttttgaaagattgtatagtctggtcattctattataattgatataatgattctttgatcatattgatcaatgtacctatttgaatcaatcgacattggattctctattcttatgtaatcccagatggatcaaggctagtcataccttaaatcacaagctgtattattacttactgtgatggataaagggactttacaaaaaacttaggacccaagtccttaaccacattctaatgtgagggagaattaaatgtacattgagactgacaatctaatatacagagtgtttgatcaagttaaaacatcaattgtcaggaattcattgataaaccaacttgttagttagaattaagaaaatataagagctaatactcgcaagtcatttgcttcttcaagagcctgtctcaactatcaagtgaatacatgatctaaaactagtatgatggctgattccaaattggtaagcctgaacaataatctatctggtaaaatcaaggatcaaggtaaagttatcaagaattattacggcacaatggacatcaagaaaattaacgatgggttattagatagtaagatacttggggcgtttaacacagtgatagctttgttgggatcaatcatcatcattgtgatgaatatcatgataattcaaaattacaccagaacgactgataatcaggcactaatcaaagagtcactccagagtgtacagcaacaaatcaaagctttaacagacaaaatcgggacagagataggccccaaagtctcactaattgacacatccagcaccatcacaattcctgctaacatagggttactgggatccaagataagtcagtctaccagcagtattaatgagaatgttaacgataaatgcaaatttactcttcctcctttaaagattcatgagtgtaatatctcttgtccgaatcctttgcctttcagagaataccgaccaatctcacaaggggtgagtgatcttgtaggactgccgaaccagatctgtctacagaagacaacatcaacaatcttaaagcccaggctgatatcctatactctaccaattaataccagagaaggggtttgcatcactgacccacttttggctgttgataatggcttcttcgcctatagccatcttgaaaagatcggatcatgtactagaggaattgcaaaacaaaggataataggggtgggtgaggtattggataggggtgataaggtgccatcaatgtttatgaccaatgtttggacaccacccaatccaagcaccatccatcattgcagctcaacttaccatgaagatttttattacacattgtgcgcagtgtcccatgtgggagatcctatccttaacagtacttcctggacagagtcactgtctctgattcgtcttgctgtaagaccaaaaagtgatagtggagactacaatcagaaatacatcgctataactaaagttgaaagagggaagtacgataaggtgatgccttacggtccatcaggtatcaagcaaggggatacattgtactttccggccgtcggttttttgccaaggaccgaatttcaatataatgactctaattgtcccataattcattgcaagtacagcaaagcagaaaactgtaggctttcaatgggtgtcaactccaaaagtcattatattttgagatcaggactattgaagtataatctatctcttggaggagacatcatactccaatttatcgagattgctgacaatagattgaccatcggttctcctagtaagatatacaattccctaggtcaacccgttttctaccaggcatcatattcttgggatacgatgattaaattaggcgatgttgataccgttgaccctctaagagtacagtggagaaataacagtgtgatttctagacctggacagtcacagtgtcctcgatttaatgtctgtcccgaggtatgctgggaagggacatataatgatgcttttctaatagaccggctaaactgggttagtgctggtgtttatttaaacagtaaccaaactgcagagaaccctgtgtttgccgtattcaaggataacgagatcctttaccaagttccactggctgaagatgacacaaatgcacaaaaaaccatcacagattgcttcttgctggagaatgtcatatggtgtatatcactagtagaaatatacgatacaggagacagtgtgataaggccaaaactatttgcagtcaagatacctgcccaatgttcagagagttgattgaccaaaagagcaataaatattatattataattatatcagtcaaattgtaaacatccttcttataatatactcaaaaaattaaaaattcccccaagaaaatactaaatgtaatcaaactcaataaaccctgaaatattgatagtttacagagatcatatgactctttgtataattctttatagaactcctaaatatcaaagagcgataaacttgctaccctgtacacttaaagcagatactgtgattaactctgtaagtttatcagataatcctattagtgacatactgagggttattcttgtataattcttctgaagatgtgattacagataatctttcaatctagtcaagtaagcctgatttcctaattataatctccctcttaggagaaggagaacgtatcaatgttcactatatactaagtcttttattttaaatttttcttattgtcttcagatgagccttattacttcatctcatcagattataaattttctaataaattaagaaaaacttaggacccaagtcctttaaacgtgcatgattgagatagtcagaaattggagctaatttgaatactagtgagtaaattaggagtagtagtattgaaccacctcatatcttatttcttctgaactgcattaaaatttgtacccaggatataacaacatggctcatgaattatctatctctgatatcatctatcctgaatgtcaccttgacagtcctatagtatcagggaaattgatttcagctatagaatatgcgcagttgagacataaccaaccgaatggggacaagagattaactgaaaatatcaaaattaacttacagggcaaaagaagaagtgtgtatatatccagacaatctagacttggaaattacattagagacaatataaagaatttgaaggaatttctacatgtatcatatcccgagtgcaataaatctttattctctctcaagtctcctggaatgacaagtaagttgagtaatatcatgaagaaatcatttaaagcatacaatatcgtgagcagaaaaatcatagagatgttacaaaacatcaccaggaatctcataactcaggaccaaaaggatgaagttttaggtatttatgagcaggacagactcagtaacatagggaaatacatgagtcaatcgcaatggtatgagtgttttctcttctggttcacaatcaagacagagatgagagctgttatcaagaactcacagaaacctaagtttagatctgactcttgtattattcatatgaaagataataacatggaaattgtaatgaacccaaatcttgtctgcatttacaaaaatgataaggatggaaagaggtgttactaccttacaccagaaattgttctaatgtgttgtgatgtcttggaaggccggatgatgatcgagacttcgataaaatctgatatcaaataccaatctttaattaccagatccaacgctttgtggacattcattgattcactcttcccgattatgggtaataggatatacaatatagtatctatgatagagcctttagtcttggcacttttacagttgaaagatgaggcgcgcatcctaagaggggcatttctacatcattgcattaaagagatccaccaagagttgattggatgtgggtttactgatcaaaaaacaagatctatctttatcgatgatcttctatcagtaatgaatattgataatatccatctgttagctgagttcttttctttcttccgaacctttggtcatccaattctcgaagccaaaaccgcagcagacaaagttagagagcatatgctagcagataaggtccttgaatacgggccaatcatgaaagcacacgcagtcttttgtggtacaattataaatgggtaccgtgacaggcatcggggagcttggcctccactttatctgccatcgcatgcatcgaaacacataataagactcaagaattctggtgaatcattaacagttgatgattgtgttaagaactgggagtcattttgtgggattcaatttgattgctttatggagttaaaattagacagtgatttgagcatgtatatgaaagataaagcactatccccaataaaggaggaatgggatagtgtctatccacgtgaagtacttaattacacgccacccagatctaccgaaccaagaagattggtggatgtatttgtcaatgatgagaatttcgatccttataatatgttagagtatgtccttacaggagattatttaactgatgaacaattcaatgtttcttatagtctaaaggagaaagaaactaaacaagcaggaagattatttgctaaaatgacatataagatgcgtgcctgtcaggtaattgctgaagctttgattgcatcgggagtgggcaagtattttaaagagaacgggatggttaaagatgaacatgagctactcaaaactttatttcaactgtcaatctcttcagtacctcgaggcaatagtcagggcagagattccgaattttccaataacacagagaaaagtctcatatcactcaagagaacaacaggtaggcttctgaataatgaagttccttgtcgaatgaacattatgtcggctttaattgacaaaaatcagtcagaccaaaagaagcataatattctcccaaacacacgaaatcgccacaagtgtgataacactagccaaacattccttgactatcatatggaattcagtccctacaagtcagacagaatggataggacagagacatctgatttctcgaaatatgatgatggaacaggaacaaaatttgacacggtgagtgctttcctaactactgacttgaagaaattctgtctcaattggcgatatgaatccatggctatttttgcagaaagacttgacgaaatttatggcttacccggtttctttaactggatgcacaaacgattagagaagtcagtcatttatgtcgcagatcctaattgcccaccagacatcgggaaacatataaaccttgatgatactcctgaagatgacatatttatccattcaccaaaaggcgggattgaaggttatagtcagaagacttggacgatcgcaacaattccctttctgtttctcagtgcctacgaaacaaatacaaggattgcagctatagttcagggagacaatgagtcaattgccataactcagaaggtccaccctaatttaccttacaaagtcaagaaagaaatttgtgcaaggcaagctcaattatattttgacaggctcagaatgaatcttagggccttaggactcaacttaaaagcaacagagaccattataagtacacacttatttgtctactctaagaaaatacattatgatggtgcagtattatcacaagccctcaaatcaatgtctaggtgttgtttttggtcagagacattggtggacgagacaagatctgcttgcagcaatattagtactaccatagctaaggcgattgagaatggattatcaagaaatgtgggttactgcatcaacgtcttgaaggtcatacagcaactgcttatttctactgagtttagcatcaacgagacattgactgctgatgtaacatctccaatatccaataatctcgattggctggtaactgcatcccgaattcccgcaccaattggaggtttcaattacttgaatttatcaagaatatttgtaagaaatataggagaccctgtgactgcatcactagccgatctaaagagaatgatagaacatgacttgatgacagataaagttctccaaaaagtgatgaatcaagaaccaggcgatgcaagcttcctagattgggctagtgatccgtattcaggaaatctaccggattcacagagtattacaaaaacgatcaagaatataacggctaggactatattgaggacatcaccgaatccaatgctgaagggtctattccatgataaatcattcgaggaagatctggagcttgctactttcctaatggatcgtagaattatattgcccagagccgcacatgaaattttagataattctttgacaggtgctagagaggaaattgctggattacttgacacaacaaaggggctaataagatcaggattgaaaaagagcgggatacaacctaagttagtgtctagactgtctaaccatgattataatcagttcctgatactcaatagacttttgtccaataaaaagagaaacgatcttatatcccctaaaacatgttcagtggatttggccaaggctcttcgttgtcatatgtggagagatcttgctcttggtcgatcaatctatggacttgaagtgcctgacgctttggaagcaatgactggaagatatattacagggagtatggaatgccaactttgtgatcaagggaatactatgtacggttggtttttcgtgccacgagattcacaattagaccaagtgaataaagagcactcatccattagagtaccttacgtcggctcaagcactgatgagaggtcagatatcaaactaggaaacgtcaagagaccgacaagagcactgagatctgccatcgaattgcaactgtttatacctgggcatacggagattctgaagaaagttggtatgaggcttggtacttggcttcccagaggggtcaacattgatatagatgtgttaaaagctataactcctgtctcaacttccaacaatctatctcacagactcagagatagatctacacagttcaagttgccaggtagtgtccttaatagggtttctagatatgtcaacataagtaatgataatttagacttcagagtggaaggtgaaaaggtcgacaccaatctaatataccaacaaacaatgctactcggcttgtcagtattggaaggtaaatttcggttgagaacagaaacagatgattataacggaatatatcatctgcatgtcagagataattgttgtgtcaaggaggttgctgatattggtggtgttaatgctgagctacctgtcccagaatacactgaagtagaaaataataggctcatatatgacccagatcctgtttctgaaattgattgtgatcgcttatcaaagcaagagtctaaggcgagagaattagactttcctctttggtccaccgaagaactccatgatgtgttagctaagactgtggctcaaacagttctcgagatcataactaaagccgataaagatgtcctcaagcaacacttagctatagactcagatgacagtatcaatagtcttataacagagtttttaatggtggatcctgagctttttgccctttatttaggtcaatcaatttcagtcaagtgggcatttgaaatccatcatagacgcccacatggacgacatacaatggtagatttgttgtctgatctcatatccaatacatcgaaacatacatacaaagtcttgtctaatgcactgtcacatcccagagtgttcaagaggtttgtgaattgcggcttgcttcttcccactcagggtccttatcttcaccaacaagattttgagaaattatctcagaacctcttaatcacatcttatatgaattatcttatgaattggtgtgacttcaaaaaattcccttttttgatagctgaacaagatgaagcagtcgtagaattaagggaagatataataacatcaaaacacctgtgtatgataattgatctgtacgcaaatcaccataagccaccttggataattgatctcaacccacaagagaagatatgtgttcttagggactttatatctaagtgccgtcatactgatgtgtcatctaggtcttggaatatcactgatctggattttatggttttttatgcttcgttgacttatctaagaagaggaataatcaagcagctcagaattaggcaagtgacagaagtaatcgacaccactacaatgctaagagataatattttagtagaaaacccgcctattaaaactggagtactagatattagaggttgcataatatataatctagaagaaatcttatcaatgaatactaagtccacgtcaagaaaggtgttcaatttgggttcaaagttgtcagttgaaaaccacaaatatagaaggataggattaaattcgtcttcttgctacaaggcattaaatctgtcaccgttgattcaacgctacctgcccgcaggttcacaacgattatttgtaggtgaagggtcaggaagtatgatgctattatatcaacagactttaggatgttcgatatccttttacaactccggtattgatggtgattatataccagggcagagagaattgagattattcccttccgaatattcaatagctgaagacgatccgtcacaatctgataagctaaaaggtctagtggtcccattatttaatggaaggcccgaaaccacgtggatagggaacttagattcatacgagtacataatcaacagaacagccggtcgtaacatcggattggtacactcagatatggaatcaggcatagacaaacaagtggaagaaattatgatagagcattcacaccttatctcaatagcaatcaatgtaatgatagaagacggtgttcttgtatctaaaatagcatttgcaccagggttccccatatccaggctactgaatatgtatcgttcctattttgggttagtattggtgtgtttcccagtgtatagtaatccagagtccacagaagtgtatctcatctgcttgcagaaaacaattaagaccataattccacctcaaaaagttcttgatcattcatatctcagtgatgaaatcaatgatcaaggtattacatctgttattttcaagatcaaaaacattcaatcaaaacaattccatgaagatctagtgaaacactatcaggtcgaacaaccgttttttgttcctagtcatatcacatgtgatgaaaagttgcttatgcaggctggtttgaaaatgaacggaccagaaatcttgaagaatgaagtgggatatgatataggatcagatatcaatacactcaggagcactattattatcttacttaatgaagctatgaactattttgacgatgagaggagtccttctcatcatctagagccctttccagtcctagagaaaacgagagtcaaaactatcatggggagagtcacgagaaaagtaactgtttattctcttataaagttgaaggaaactaaaagcccggaactttataacatcaaaaattacataaggagaaaagttttgattctagacttcagatcccatactatgattaaactgctacctaaaggaatgaaagagagaagagagaaaagcggattcaaagaaatttggatatttgacttatctaatagggaagtcaaaatctggtggaagatcataggttacctctctttagtctgaatcgaactaccacacccatcaccaatgaagcccgaacaagtctacatagatcagttattaagaaaaacttaataacgatctctctttacccttgttcggt

SEQ ID NO: 34 - protein, 532 aa, Hendra virus
MSDIFDEAASFRSYQSKLGRDGRASAATATLTTKIRIFVPATNSPELRWELTLFALDVIRSPSAAESMKIGAAFTLISMYSERPGALIRSLLNDPDIEAVIIDVGSMLNGIPVMERRGDKAQEEMEGLMRILKTARESSKGKTPFVDSRAYGLRITDMSTLVSAVITIEAQIWILIAKAVTAPDTAEESETRRWAKYVQQKRVNPFFALTQQWLTEMRNLLSQSLSVRKFMVEILMEVKKGGSAKGRAVEIISDIGNYVEETGMAGFFATIRFGLETRYPALALNEFQSDLNTIKGLMLLYREIGPRAPYMVLLEESIQTKFAPGGYPLLWSFAMGVATTIDRSMGALNINRGYLEPMYFRLGQKSARHHAGGIDQNMANKLGLNSDQVAELAAAVQETSVGRQDNNMQAREAKFAAGGVLVGGGEQDIDEEEEPIEHSGRQSVTFKREMSMSSLADSVPSSSVSTSGGTRLTNSLLNLRSRLAAKAIKESTAQSSSERNPPNNRPQADSGRKDDQEPKPAQNDLDFVRADV

SEQ ID NO: 35 - protein, 707 aa, Hendra virus
MDKLDLVNDGLDIIDFIQKNQKEIQKTYGRSSIQQPSTKDRTRAWEDFLQSTSGEHEQAEGGMPKNDGGTEGRNVEDLSSVTSSDGTIGQRVSNTRAWAEDPDDIQLDPMVTDVVYHDHGGECTGHGPSSSPERGWSYHMSGTHDGNVRAVPDTKVLPNAPKTTVPEEVREIDLIGLEDKFASAGLNPAAVPFVPKNQSTPTEEPPVIPEYYYGSGRRGDLSKSPPRGNVNLDSIKIYTSDDEDENQLEYEDEFAKSSSEVVIDTTPEDNDSINQEEVVGDPSDQGLEHPFPLGKFPEKEETPDVRRKDSLMQDSCKRGGVPKRLPMLSEEFECSGSDDPIIQELEREGSHPGGSLRLREPPQSSGNSRNQPDRQLKTGDAASPGGVQRPGTPMPKSRIMPIKKGTDAKSQYVGTEDVPGSKSGATRYVRGLPPNQESKSVTAENVQLSAPSAVTRNEGHDQEVTSNEDSLDDKYIMPSDDFANTFLPHDTDRLNYHADHLNDYDLETLCEESVLMGIVNAIKLINIDMRLNHIEEQMKEIPKIINKIDSIDRVLAKTNTALSTIEGHLVSMMIMIPGKGKGERKGKTNPELKPVIGRNILEQQELFSFDNLKNFRDGSLTDEPYGGVARIRDDLILPELNFSETNASQFVPLADDASKDVVRTMIRTHIKDRELRSELMDYLNRAETDEEVQEVANTVNDIIDGNI

SEQ ID NO: 36 - protein, 457 aa, Hendra virus
MDKLDLVNDGLDIIDFIQKNQKEIQKTYGRSSIQQPSTKDRTRAWEDFLQSTSGEHEQAEGGMPKNDGGTEGRNVEDLSSVTSSDGTIGQRVSNTRAWAEDPDDIQLDPMVTDVVYHDHGGECTGHGPSSSPERGWSYHMSGTHDGNVRAVPDTKVLPNAPKTTVPEEVREIDLIGLEDKFASAGLNPAAVPFVPKNQSTPTEEPPVIPEYYYGSGRRGDLSKSPPRGNVNLDSIKIYTSDDEDENQLEYEDEFAKSSSEVVIDTTPEDNDSINQEEVVGDPSDQGLEHPFPLGKFPEKEETPDVRRKDSLMQDSCKRGGVPKRLPMLSEEFECSGSDDPIIQELEREGSHPGGSLRLREPPQSSGNSRNQPDRQLKTGDAASPGGVQRPGTPMPKSRIMPIKKGHRREVSICWDGRRAWVEEWCNPVCSRITPQPRKQECYCGECPTECSQCCHEE

SEQ ID NO: 37 - protein, 166 aa, Hendra virus
MMASILLTLFRRTKKKYKRHTDDQASNNQVPKTGQEHGRTSCRAPVENMNRLRGECLRMMEVLKEEMWRIYPVLLPQMELLDKECQTPELGQKTQMTYNWTQWLQTLYTMIMEENVPDMDLLQALREGGVITCQEHTMGMYVLYLIQRCCPMLPKLQFLKKLGKLI

SEQ ID NO: 38 - protein, 352 aa, Hendra virus
MDFSVSDNLDDPIEGVSDFSPTSWENGGYLDKVEPEIDKHGSMIPKYKIYTPGANERKFNNYMYMICYGFVEDVERSPESGKRKKIRTIAAYPLGVGKSTSHPQDLLEELCSLKVTVRRTAGATEKIVFGSSGPLHHLLPWKKILTGGSIFNAVKVCRNVDQIQLENQQSLRIFFLSITKLNDSGIYMIPRTMLEFRRNNAIAFNLLVYLKIDADLAKAGIQGSFDKDGTKVASFMLHLGNFVRRAGKYYSVEYCKRKIDRMKLQFSLGSIGGLSLHIKINGVISKRLFAQMGFQKNLCFSLMDINPWLNRLTWNNSCEISRVAAVLQPSVPREFMIYDDVFIDNTGKILKG

SEQ ID NO: 39 - protein, 546 aa, Hendra virus
MATQEVRLKCLLCGIIVLVLSLEGLGILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT

SEQ ID NO: 40 - protein, 604 aa, Hendra virus
MMADSKLVSLNNNLSGKIKDQGKVIKNYYGTMDIKKINDGLLDSKILGAFNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 41 - protein, 2244 aa, Hendra virus
MAHELSISDIIYPECHLDSPIVSGKLISAIEYAQLRHNQPNGDKRLTENIKINLQGKRRSVYISRQSRLGNYIRDNIKNLKEFLHVSYPECNKSLFSLKSPGMTSKLSNIMKKSFKAYNIVSRKIIEMLQNITRNLITQDQKDEVLGIYEQDRLSNIGKYMSQSQWYECFLFWFTIKTEMRAVIKNSQKPKFRSDSCIIHMKDNNMEIVMNPNLVCIYKNDKDGKRCYYLTPEIVLMCCDVLEGRMMIETSIKSDIKYQSLITRSNALWTFIDSLFPIMGNRIYNIVSMIEPLVLALLQLKDEARILRGAFLHHCIKEIHQELIGCGFTDQKTRSIFIDDLLSVMNIDNIHLLAEFFSFFRTFGHPILEAKTAADKVREHMLADKVLEYGPIMKAHAVFCGTIINGYRDRHRGAWPPLYLPSHASKHIIRLKNSGESLTVDDCVKNWESFCGIQFDCFMELKLDSDLSMYMKDKALSPIKEEWDSVYPREVLNYTPPRSTEPRRLVDVFVNDENFDPYNMLEYVLTGDYLTDEQFNVSYSLKEKETKQAGRLFAKMTYKMRACQVIAEALIASGVGKYFKENGMVKDEHELLKTLFQLSISSVPRGNSQGRDSEFSNNTEKSLISLKRTTGRLLNNEVPCRMNIMSALIDKNQSDQKKHNILPNTRNRHKCDNTSQTFLDYHMEFSPYKSDRMDRTETSDFSKYDDGTGTKFDTVSAFLTTDLKKFCLNWRYESMAIFAERLDEIYGLPGFFNWMHKRLEKSVIYVADPNCPPDIGKHINLDDTPEDDIFIHSPKGGIEGYSQKTWTIATIPFLFLSAYETNTRIAAIVQGDNESIAITQKVHPNLPYKVKKEICARQAQLYFDRLRMNLRALGLNLKATETIISTHLFVYSKKIHYDGAVLSQALKSMSRCCFWSETLVDETRSACSNISTTIAKAIENGLSRNVGYCINVLKVIQQLLISTEFSINETLTADVTSPISNNLDWLVTASRIPAPIGGFNYLNLSRIFVRNIGDPVTASLADLKRMIEHDLMTDKVLQKVMNQEPGDASFLDWASDPYSGNLPDSQSITKTIKNITARTILRTSPNPMLKGLFHDKSFEEDLELATFLMDRRIILPRAAHEILDNSLTGAREEIAGLLDTTKGLIRSGLKKSGIQPKLVSRLSNHDYNQFLILNRLLSNKKRNDLISPKTCSVDLAKALRCHMWRDLALGRSIYGLEVPDALEAMTGRYITGSMECQLCDQGNTMYGWFFVPRDSQLDQVNKEHSSIRVPYVGSSTDERSDIKLGNVKRPTRALRSAIELQLFIPGHTEILKKVGMRLGTWLPRGVNIDIDVLKAITPVSTSNNLSHRLRDRSTQFKLPGSVLNRVSRYVNISNDNLDFRVEGEKVDTNLIYQQTMLLGLSVLEGKFRLRTETDDYNGIYHLHVRDNCCVKEVADIGGVNAELPVPEYTEVENNRLIYDPDPVSEIDCDRLSKQESKARELDFPLWSTEELHDVLAKTVAQTVLEIITKADKDVLKQHLAIDSDDSINSLITEFLMVDPELFALYLGQSISVKWAFEIHHRRPHGRHTMVDLLSDLISNTSKHTYKVLSNALSHPRVFKRFVNCGLLLPTQGPYLHQQDFEKLSQNLLITSYMNYLMNWCDFKKFPFLIAEQDEAVVELREDIITSKHLCMIIDLYANHHKPPWIIDLNPQEKICVLRDFISKCRHTDVSSRSWNITDLDFMVFYASLTYLRRGIIKQLRIRQVTEVIDTTTMLRDNILVENPPIKTGVLDIRGCIIYNLEEILSMNTKSTSRKVFNLGSKLSVENHKYRRIGLNSSSCYKALNLSPLIQRYLPAGSQRLFVGEGSGSMMLLYQQTLGCSISFYNSGIDGDYIPGQRELRLFPSEYSIAEDDPSQSDKLKGLVVPLFNGRPETTWIGNLDSYEYIINRTAGRNIGLVHSDMESGIDKQVEEIMIEHSHLISIAINVMIEDGVLVSKIAFAPGFPISRLLNMYRSYFGLVLVCFPVYSNPESTEVYLICLQKTIKTIIPPQKVLDHSYLSDEINDQGITSVIFKIKNIQSKQFHEDLVKHYQVEQPFFVPSHITCDEKLLMQAGLKMNGPEILKNEVGYDIGSDINTLRSTIIILLNEAMNYFDDERSPSHHLEPFPVLEKTRVKTIMGRVTRKVTVYSLIKLKETKSPELYNIKNYIRRKVLILDFRSHTMIKLLPKGMKERREKSGFKEIWIFDLSNREVKIWWKIIGYLSLV

SEQ ID NO: 42 - protein, 707 aa, Hendra virus
MDKLDLVNDGLDIIDFIQKNQKEIQKTYGRSSIQQPSTKDRTRAWEDFLQSTSGEHEQAEGGMPKNDGGTEGRNVEDLSSVTSSDGTIGQRVSNTRAWAEDPDDIQLDPMVTDVVYHDHGGECTGHGPSSSPERGWSYHMSGTHDGNVRAVPDTKVLPNAPKTTVPEEVREIDLIGLEDKFASAGLNPAAVPFVPKNQSTPTEEPPVIPEYYYGSGRRGDLSKSPPRGNVNLDSIKIYTSDDEDENQLEYEDEFAKSSSEVVIDTTPEDNDSINQEEVVGDPSDQGLEHPFPLGKFPEKEETPDVRRKDSLMQDSCKRGGVPKRLPMLSEEFECSGSDDPIIQELEREGSHPGGSLRLREPPQSSGNSRNQPDRQLKTGDAASPGGVQRPGTPMPKSRIMPIKKGTDAKSQYVGTEDVPGSKSGATRYVRGLPPNQESKSVTAENVQLSAPSAVTRNEGHDQEVTSNEDSLDDNILCHQMILLTLFLPHDTDRLNYHADHLNDYDLETLCEESVLMGIVNAIKLINIDMRLNHIEEQMKEIPKIINKIDSIDRVLAKTNTALSTIEGHLVSMMIMIPGKGKGERKGKTNPELKPVIGRNILEQQELFSFDNLKNFRDGSLTDEPYGGVARIRDDLILPELNFSETNASQFVPLADDASKDVVRTMIRTHIKDRELRSELMDYLNRAETDEEVQEVANTVNDIIDGNI

SEQ ID NO: 43 - protein, 457 aa, Hendra virus; identical to SEQ ID NO: 36

SEQ ID NO: 44 - protein, 166 aa, Hendra virus; identical to SEQ ID NO: 37

SEQ ID NO: 45 - DNA, 2698 bp, Hendra virus
aggatccaagaccataaatctaggatcctttacaatctcagaccctggtgcaaggttatatatagagcccaattctcctaattaaacagtgtctcaggttgacaaatggacaagttggatctagtcaatgatggcctcgatattattgactttattcagaagaaccaaaaagaaatacaaaagacatacggacgatcaagcatccaacaaccaagtaccaaagacaggacaagagcatgggaggacttcttgcagagcaccagtggagaacatgaacaggctgaggggggaatgcctaagaatgatggaggtactgaaggaagaaatgtggaggatctatccagtgttacttcctcagatggaactattggacaaagagtgtcaaacacccgagcttgggcagaagacccagatgacatacaactggacccaatggttacagacgttgtataccatgatcatggaggagaatgtaccggacatggaccttcttcaagccctgagagagggtggagttatcacatgtcaggaacacacgatgggaatgtacgtgctgtacctgatacaaaggtgttgcccaatgctcccaaaactacagttcctgaagaagttagggaaattgatttaattgggttggaggacaaatttgcatcagcgggattaaatccagctgcagttccctttgtccccaagaatcaatcaaccccaactgaggagcctccagtcatcccggagtattactatggatctgggaggagaggagatctctcaaaatctcctcctagaggtaatgtcaatctggacagcatcaagatttacacttcggacgacgaggatgaaaatcagctggagtatgaggatgagtttgccaaaagctcaagtgaggttgtcattgacactactcctgaggacaatgattctatcaaccaggaggaagtagttggggacccgtctgatcagggtttagagcatcctttccctttggggaaattcccggagaaagaagaaactcctgatgtacgcagaaaggattccctaatgcaggacagttgtaagagaggaggagtaccgaaaagactgcccatgttatctgaagagttcgagtgctccggatcagatgatccaataatacaagaattagaacgagaagggtctcatccaggaggttcattacgtttgagggaaccacctcagtcgtcagggaattccagaaatcaacctgaccgtcagctgaagacaggtgatgcagcatcacccggaggcgtccagagaccaggcacaccgatgcctaaatcaaggatcatgcccattaaaaagggcacagacgcgaagtctcaatatgttgggacggaagacgtgcctgggtcgaagagtggtgcaacccggtatgttcgcggattaccccccaaccaagaaagcaagagtgttactgcggagaatgtccaactgagtgctcccagtgctgtcacgaggaatgaaggacacgaccaggaagttaccagcaacgaagatagtctggatgacaatatattatgccatcagatgattttgctaacacttttcttaccccatgacactgataggttaaattatcatgcagaccatctcaatgattacgatttagagaccttgtgtgaggagtcagtattgatgggcatcgttaacgcaatcaaactcatcaacattgatatgaggttgaatcacattgaggaacagatgaaagagatacccaagatcatcaacaaaatagactccattgatcgagtgttggctaagaccaatactgcattgtcgacaatagaaggacacctagtctcgatgatgatcatgataccagggaaaggcaagggtgaaaggaaagggaaaacaaatccggagctaaaacctgtaattgggagaaatatcctagaacagcaagagttattttcattcgacaatctcaagaatttcagagatgggtcattgactgatgagccctatggaggggtggcccgaataagagatgatttgatcctgcctgaactcaacttcagtgagacaaatgcatcacaatttgttcctttggcagatgatgcatctaaggatgtcgtgagaactatgattaggactcacatcaaggacagagagttgaggtctgagttgatggattatctaaatcgagcagaaacagatgaagaggttcaggaggtggccaatacagtcaatgatattattgatgggaacatctaaacatgtcagtaatttgatagagtagcagtcgaaccaagatcatatattagaggtacacatcataatggccagtgtcttaatctcaaccattcataaaccgtcagtgtttcccttataaactccattatgtagtttattaatatctagagctattctttattaagtaataaatctgtattagattattatataatctctataatcaacacgcctattacttcatgcacttactatcaccatttaataatagttcagatcaccccttcaaaatctgggttgtgccaagtcattttcctcaccatataatccacgcagtatgcattcaaatactaaaccacattcagacagagttagacattcaagcaatcaataactatattaactataagaatgatgaatcagccctgcgccaacaaggcaatgtcaatcacttgaatgcaggggtagaccccagaatccattaagaaaa